SEQUENCE LISTING (25 sequences)

SEQ ID NO: 1 - protein, 293 aa, unidentified
MKGLLSVAALSLAVSEVSAHYIFQQLSTGSTKHGVFQYIRQNTNYNSPVTDLSSNDLRCNEGGASGANTQTVTVRAGDSFTFHLDTPVYHQGPVSVYLSKAPGSASSYDGSGTWFKIKDWGPTFPGGQWTLAGSYTAQLPSCITDGEYLLRIQSLGIHNPYPAGTPQFYISCAQIKVTGGGSVNPSGVAIPGAFKATDPGYTANIYSNFNSYTVPGPSVFSCGSNGGGSSPVEPQPQPTTTLVTSTRAPVATQPAGCAVAKWGQCGGNGWTGCTTCAAGSTCNTQNAYYHQCV

SEQ ID NO: 2 - protein, 242 aa, Bacillus subtilis
MPYLKRVLLLLVTGLFMSLFAVTATASAQTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 3 - protein, 948 aa, unidentified
MGASIKTSIKIRTVAFVSIIAIALSILSFIPNRAYASPQRGRPRLNAARTTFVGDNGQPLRGPYTSTEWTAAAPYDQIARVKELGFNAVHLYAECFDPRYPAPGSKAPGYAVNEIDKIVERTRELGLYLVITIGNGANNGNHNAQWARDFWKFYAPRYAKETHVLYEIHNEPVAWGPPYSSSTANPPGAVDMEIDVYRIIRTYAPETPVLLFSYAVFGGKGGAAEALKDIRAFNKAVFGNENAVWTNEAVAFHGYAGWQETTIAVEELLKAGYPCFMTEYAGGAWGSGMGGLDVELTYELERLGVSWLTFQYIPPTGVSDDVTKPEYFSALVENSGLSWTPDYGNWPAARGVYGNGGLARETATWINNFLTGTTRIEAEDFDWGGNGVSYYDTDSVNVGGQYRPDEGVDIEKTSDTGGGYNVGWISEGEWLEYTIRVRNPGYYNLSLRVAGISGSRVQVSFGNQDKTGVWELPATGGFQTWTTATRQVFLGAGLQKLRINALSGGFNLNWIELSPISTGTIPDGTYKFLNRANGKTLQEVTGNNSIITADYKGITEQHWKIQHIGGGQYRISSAGRGWNWNWWMGFGTVGWWGTGSSTCFIISPTGDGYYRIVLVGDGTNLQISSGDPSKIEGKAFHGGANQQWAILPVSAPAFPTGLSAVLDSSGNTANLTWNAAPGANSYNVKRSTKSGGPYTTIATNITSTNYTDTGVATGTKYYYVVSAVSNGVETLNSAEAILQYPKLTGTVIGTQGSWNNIGNTIHKAFDGDLNTFFDGPTANGCWLGLDFGEGVRNVITQIKFCPRSGYEQRMIGGIFQGANKEDFSDAVTLFTITSLPGSGTLTSVDVDNPTGFRYVRYLSPDGSNGNIAELQFFGTPAGEENDDVHLGDINDDGNINSTDLQMLKRHLLRSIRLTEKQLLNADTNRDGRVDSTDLALLKRYILRVITTL

SEQ ID NO: 4 - protein, 443 aa, Gonapodya prolifera
MARLSSLIALVLAFVAVSAPALAARGRPRLNGKTFVADSGVPLRGPFTSTEWTPAVPAANIANMRNYNFNAIHLYAETFDPNYPAAGSQKPGYAATRVDQIVAATKAANMYVVIVLANGANNGKFNLNYAKDFWSFYAARYKNETHVIYEIHNEPVQWGPPYISSTQSPGAVSMNADCYKIIRAVAPDTPVLLFTYASIGGGSSAAGAVKDAQSFNTAVFGNANAQWTNEAIAIHGYWGAQGASDAAKALNAAGFSVVLTEFAAATSPTSPNGGQDTVLTGFMEQQGVSWLTFLHVPPTGVSGDVTDPNQYTNRMTAAGIGFDRDPGLNAVGGGQAAPVPVPAPAPVPSPVPAPVPAVPAVRTTTARPAPSPSPVPAPVPAPAPVPAPVPAPVPAPVPAPVPAPVPASPAATTTRRHRTRPPRTTTAPAVPAPPPAATPKVCG

SEQ ID NO: 5 - protein, 413 aa, Dickeya chrysanthemi
MNGNVSLWVRHCLHAALFVSATAGSFSVYADTVKIDANVNYQIIQGFGGMSGVGWINDLTTEQINTAYGSGVGQIGLSIMRVRIDPDSSKWNIQLPSARQAVSLGAKIMATPWSPPAYMKSNNSLINGGRLLPANYSAYTSHLLDFSKYMQTNGAPLYAISIQNEPDWKPDYESCEWSGDEFKSYLKSQGSKFGSLKVIVAESLGFNPALTDPVLKDSDASKYVSIIGGHLYGTTPKPYPLAQNAGKQLWMTEHYVDSKQSANNWTSAIEVGTELNASMVSNYSAYVWWYIRRSYGLLTEDGKVSKRGYVMSQYARFVRPGALRIQATENPQSNVHLTAYKNTDGKMVIVAVNTNDSDQMLSLNISNANVTKFEKYSTSASLNVEYGGSSQVDSSGKATVWLNPLSVTTFVSK

SEQ ID NO: 6 - protein, 422 aa, Bacillus subtilis
MIPRIKKTICVLLVCFTMLSVMLGPGATEVLAASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 7 - protein, 547 aa, Bacteroides ovatus
MKNITLLFCLFLANILLGACSGGEDEKKEMDEGKGAYALFLKKSITVSTGESQTDVVVEWAKTSWEITLGEGDIVKSVTPTSGGSNTGEKQYTKVRVSCGANSTMKKRTQTIHLFDKTNETTVDLLVEQEPPFKSVTLTVDPSVKYQPVVGFGGMYNPKIWCGDNLISASQLDKMYGAGGLGYSILRLMIYPNESDWSADVEAAKAAQANGAIIFACPWDCTDALADKITVNGKEMKHLKKENYEAYANHLIRYVTFMKEKGVNLYAISVQNEPDMEFTYWTPSEVVDFVKQYGARIRETGVKLMSPEACGMQPEYTDPIINNAEAFAQTDILAGHLYQGFTDLSSGYVKNRHDYICGVYSRIQGKTWWMTEHLFNDGENSDDSSKWEFLKWQYSLNHLGKEIHMCMEGYCSAYIYWYLKRFYGLMGDTDKRSPTSEGEITKNGYIMAHYAQYATETTRIKVVTNNEEVCATAYWDEKTGEVTIVLLNLNGASQWLEIPLAGIKKASAVETNETKNMEVIDTGLMESAEGITVLLSANSITSVRLTF

SEQ ID NO: 8 - protein, 502 aa, Bacteroides ovatus
MEKQSFSDGLFSPLGIKRVIFMLVLLTTSFISCSNSDEKGGSLEVAQEYRNLEFDARGSRQTIQIDGPAEWHISTSESWCKSSHTIGEGKQYVNITVEANDTQKERTATVTVSASGAPDIIINVKQSLYSVPAYDEYIAPDNTGMRDLTSMQLSALMKAGVNVGNTFEAVIVGNDGSLSGDETCWGNPTPNKVLFEGIKAAGFDVVRIPVAYSHQFEDAATYKIKSAWMDKVEAAVKAALDAGLYVIINIHWEGGWLNHPVDANKEALDERLEAMWKQIALRFRDYDDRLLFAGTNEVNNDDANGAQPTEENYRVQNGFNQVFVNTVRATGGRNHYRHLIVQAYNTDVAKAVAHFTMPLDIVQNRIFLECHYYDPYDFTIMPNDENFKSQWGAAFAGGDVSATGQEGDIEATLSSLNVFINNNVPVIIGEYGPTLRDQLTGEALENHLKSRNDYIEYVVKTCVKNKLVPLYWDAGYTEKLFDRTTGQPHNAASIAAIMKGLN

SEQ ID NO: 9 - protein, 838 aa, unidentified
MKVSRVLALVLGAVIPAHAAFSWKNVKLGGGGGFVPGIIFHPKTKGVAYARTDIGGLYRLNADDSWTAVTDGIADNAGWHNWGIDAVALDPQDDQKVYAAVGMYTNSWDPSNGAIIRSSDRGATWSFTNLPFKVGGNMPGRGAGERLAVDPANSNIIYFGARSGNGLWKSTDGGVTFSKVSSFTATGTYIPDPSDSNGYNSDKQGLMWVTFDSTSSTTGGATSRIFVGTADNITASVYVSTNAGSTWSAVPGQPGKYFPHKAKLQPAEKALYLTYSDGTGPYDGTLGSVWRYDIAGGTWKDITPVSGSDLYFGFGGLGLDLQKPGTLVVASLNSWWPDAQLFRSTDSGTTWSPIWAWASYPTETYYYSISTPKAPWIKNNFIDVTSESPSDGLIKRLGWMIESLEIDPTDSNHWLYGTGMTIFGGHDLTNWDTRHNVSIQSLADGIEEFSVQDLASAPGGSELLAAVGDDNGFTFASRNDLGTSPQTVWATPTWATSTSVDYAGNSVKSVVRVGNTAGTQQVAISSDGGATWSIDYAADTSMNGGTVAYSADGDTILWSTASSGVQRSQFQGSFASVSSLPAGAVIASDKKTNSVFYAGSGSTFYVSKDTGSSFTRGPKLGSAGTIRDIAAHPTTAGTLYVSTDVGIFRSTDSGTTFGQVSTALTNTYQIALGVGSGSNWNLYAFGTGPSGARLYASGDSGASWTDIQGSQGFGSIDSTKVAGSGSTAGQVYVGTNGRGVFYAQGTVGGGTGGTSSSTKQSSSSTSSASSSTTLRSSVVSTTRASTVTSSRTSSAAGPTGSGVAGHYAQCGGIGWTGPTQCVAPYVCQKQNDYYYQCV

SEQ ID NO: 10 - protein, 513 aa, unidentified
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 11 - protein, 471 aa, unidentified
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 12 - protein, 239 aa, unidentified
MKLPVTLAMLAATAMGQTMCSQYDSASSPPYSVNQNLWGEYQGTGSQCVYVDKLSSSGASWHTEWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 13 - protein, 416 aa, unidentified
MRISNLIVAASAASMVSALPSRQMKKRDSGFKWVGTSESGAEFGSALPGTLGTDYTWPETSKIQVLRNKGMNIFRIPFLMERLTPDGLTSSFASTYLSDLKSTVEFVTNSGAYAVLDPHNYGRFDGSIITSTSDFKTWWKNVATEFADNDKVIFDTNNEYHDMEQSLVLDLNQAAINGIRAAGATTQYIFVEGNAYTGAWDWTTYNDNLSGLTDSEDKIIYEMHQYLDSDSSGTSETCVSSTIGQERLEKATEWLKTNNKQGIVGEFAGGVNSVCEEAVEGMLAYMSENSDVWVGASWWSAGPWWGTYMYSLEPTDGTAYSTYLPILEKYFPSGDASASSSASVSVAAATSTASTTTAAFEQTTTPATQGPSATNSAGEVNQYYQCGGINWTGPTVCASPYTCKVQNDYYYQCVAE

SEQ ID NO: 14 - protein, 331 aa, unidentified
MKFQSTLLLAAAAGSALAVPHGSGHKKRASVFEWFGSNESGAEFGTNIPGVWGTDYIFPDPSTISTLIGKGMNFFRVQFMMERLLPDSMTGSYDEEYLANLTTVVKAVTDGGAHALIDPHNYGRYNGEIISSTSDFQTFWQNLAGQYKDNDLVMFDTNNEYYDMDQDLVLNLNQAAINGIRAAGASQYIFVEGNSWTGAWTWVDVNDNMKNLTDPEDKIVYEMHQYLDSDGSGTSETCVSGTIGKERITDATQWLKDNKKVGFIGEYAGGSNDVCRSAVSGMLEYMANNTDVWKGASWWAAGPWWGDYIFSLEPPDGTAYTGMLDILETYL

SEQ ID NO: 15 - protein, 465 aa, unidentified
MKSSISVVLALLGHSAAWSYATKSQYRANIKINARQTYQTMIGGGCSGAFGIACQQFGSSGLSPENQQKVTQILFDENIGGLSIVRNDIGSSPGTTILPTCPATPQDKFDYVWDGSDNCQFNLTKTALKYNPNLYVYADAWSAPGCMKTVGTENLGGQICGVRGTDCKHDWRQAYADYLVQYVRFYKEEGIDISLLGAWNEPDFNPFTYESMLSDGYQAKDFLEVLYPTLKKAFPKVDVSCCDATGARQERNILYELQQAGGERYFDIATWHNYQSNPERPFNAGGKPNIQTEWADGTGPWNSTWDYSGQLAEGLQWALYMHNAFVNSDTSGYTHWWCAQNTNGDNALIRLDRDSYEVSARLWAFAQYFRFARPGSVRIGATSDVENVYVTAYVNKNGTVAIPVINAAHFPYDLTIDLEGIKKRKLSEYLTDNSHNVTLQSRYKVSGSSLKVTVEPRAMKTFWLE

SEQ ID NO: 16 - protein, 211 aa, unidentified
MKVTAAFAGLLVTAFAAPVPEPVLVSRSAGINYVQNYNGNLGDFTYDESAGTFSMYWEDGVSSDFVVGLGWTTGSSKAITYSAEYSASGSSSYLAVYGWVNYPQAEYYIVEDYGDYNPCSSATSLGTVYSDGSTYQVCTDTRTNEPSITGTSTFTQYFSVRESTRTSGTVTVANHFNFWAQHGFGNSDFNYQVMAVEAWSGAGSASVTISS

SEQ ID NO: 17 - protein, 437 aa, unidentified
MMMLSKSLLSAATAASALAAVLQPVPRASSFVTISGTQFNIDGKVGYFAGTNCYWCSFLTNHADVDSTFSHISSSGLKVVRVWGFNDVNTQPSPGQIWFQKLSATGSTINTGADGLQTLDYVVQSAEQHNLKLIIPFVNNWSDYGGINAYVNAFGGNATTWYTNTAAQTQYRKYVQAVVSRYANSTAIFAWELGNEPRCNGCSTDVIVQWATSVSQYVKSLDSNHLVTLGDEGLGLSTGDGAYPYTYGEGTDFAKNVQIKSLDFGTFHLYPDSWGTNYTWGNGWIQTHAAACLAAGKPCVFEEYGAQQNPCTNEAPWQTTSLTTRGMGGDMFWQWGDTFANGAQSNSDPYTVWYNSSNWQCLVKNHVDAINGGTTTPPPVSSTTTTSSRTSSTPPPPGGSCSPLYGQCGGSGYTGPTCCAQGTCIYSNYWYSQCLNT

SEQ ID NO: 18 - protein, 335 aa, unidentified
MFAKLSLLSLLFSSAALGASNQTLSYGNIDKSATPEARALLKYIQLQYGSHYISGQQDIDSWNWVEKNIGVAPAILGSDFTYYSPSAVAHGGKSHAVEDVIQHAGRNGINALVWHWYAPTCLLDTAKEPWYKGFYTEATCFNVSEAVNDHGNGTNYKLLLRDIDAIAAQIKRLDQAKVPILFRPLHEPEGGWFWWGAQGPAPFKKLWDILYDRITRYHNLHNMVWVCNTADPAWYPGNDKCDIATIDHYPAVGDHGVAADQYKKLQTVTNNERVLAMAEVGPIPDPDKQARENVNWAYWMVWSGDFIEDGKQNPNQFLHKVYNDTRVVALNWEGA

SEQ ID NO: 19 - protein, 383 aa, unidentified
MKLSNALLTLASLALANVSTALPKASPAPSTSSSAASTSFASTSGLQFTIDGETGYFAGTNSYWIGFLTDNADVDLVMGHLKSSGLKILRVWGFNDVTSQPSSGTVWYQLHQDGKSTINTGADGLQRLDYVVSSAEQHDIKLIINFVNYWTDYGGMSAYVSAYGGSGETDFYTSDTMQSAYQTYIKTVVERYSNSSAVFAWELANEPRCPSCDTSVLYNWIEKTSKFIKGLDADRMVCIGDEGFGLNIDSDGSYPYQFSEGLNFTMNLGIDTIDFGTLHLYPDSWGTSDDWGNGWITAHGAACKAAGKPCLLEEYGVTSNHCSVEGSWQKTALSTTGVGADLFWQYGDDLSTGKSPDDGNTIYYGTSDYQCLVTDHVAAIGSA

SEQ ID NO: 20 - protein, 452 aa, unidentified
MHQRALLFSALLTAVRAQQAGTLTEEVHPSLTWQKCTSEGSCTEQSGSVVIDSNWRWTHSVNDSTNCYTGNTWDATLCPDDETCAANCALDGADYESTYGVTTDGDSLTLKFVTGSNVGSRLYLMDTSDEGYQTFNLLDAEFTFDVDVSNLPCGLNGALYFTAMDADGGVSKYPANKAGAKYGTGYCDSQCPRDLKFIDGQANVDGWEPSSNNDNTGIGNHGSCCPEMDIWEANKISTALTPHPCDSSEQTMCEGNDCGGTYSDDRYGGTCDPDGCDFNPYRMGNDSFYGPGKTIDTGSKMTVVTQFITDGSGSLSEIKRYYVQNGNVIANADSNISGVTGNSITTDFCTAQKKAFGDEDIFAEHNGLAGISDAMSSMVLILSLWDDYYASMEWLDSDYPENATATDPGVARGTCDSESGVPATVEGAHPDSSVTFSNIKFGPINSTFSASA

SEQ ID NO: 21 - protein, 536 aa, unidentified
MSSFQIYRAALLLSILATANAQQVGTYTTETHPSLTWQTCTSDGSCTTNDGEVVIDANWRWVHSTSSATNCYTGNEWDTSICTDDVTCAANCALDGATYEATYGVTTSGSELRLNFVTQGSSKNIGSRLYLMSDDSNYELFKLLGQEFTFDVDVSNLPCGLNGALYFVAMDADGGTSEYSGNKAGAKYGTGYCDSQCPRDLKFINGEANCDGWEPSSNNVNTGVGDHGSCCAEMDVWEANSISNAFTAHPCDSVSQTMCDGDSCGGTYSASGDRYSGTCDPDGCDYNPYRLGNTDFYGPGLTVDTNSPFTVVTQFITDDGTSSGTLTEIKRLYVQNGEVIANGASTYSSVNGSSITSAFCESEKTLFGDENVFDKHGGLEGMGEAMAKGMVLVLSLWDDYAADMLWLDSDYPVNSSASTPGVARGTCSTDSGVPATVEAESPNAYVTYSNIKFGPIGSTYSSGSSSGSGSSSSSSSTTTKATSTTLKTTSTTSSGSSSTSAAQAYGQCGGQGWTGPTTCVSGYTCTYENAYYSQCL

SEQ ID NO: 22 - protein, 744 aa, unidentified
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 23 - protein, 344 aa, unidentified
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 24 - protein, 459 aa, unidentified
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 25 - protein, 418 aa, unidentified
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

(19) United States
(12) Patent Application Publication
(10) Pub. No.: US 2025/0331544 A1
(43) Pub. Date: Oct. 30, 2025

(54) METHODS OF TREATING BIOMASS TO PRODUCE OLIGOSACCHARIDES AND RELATED COMPOSITIONS

(71) Applicant: Cambridge Glycoscience Ltd., Cambridge (GB)

(72) Inventors: Thomas J. Simmons, Cambridge (GB); Jeremy Bartosiak-Jentys, Chester (GB); Aleksandra Wlodek, Sawston (GB); Gustavo Valente, Newcastle upon Tyne (GB)

(21) Appl. No.: 18/981,243

(22) Filed: Dec. 13, 2024

Related U.S. Application Data
(63) Continuation of application No. 17/691,931, filed on Mar. 10, 2022, now abandoned.
Application No. 17/691,931 is a continuation of application No. 17/083,121, filed on Oct. 28, 2020, now Pat. No. 11,297,865.
Application No. 17/083,121 is a continuation of application No. PCT/EP2020/072929, filed on Aug. 14, 2020.

(30) Foreign Application Priority Data
Aug. 16, 2019 (GB) 1911762.1
Aug. 16, 2019 (GB) 1911764.7
Feb. 19, 2020 (GB) 2002315.6

(51) Int. Cl. A23L 29/30; A23L 7/126; A23L 7/135; A23L 27/30; A23L 27/60; A23L 29/262; C12P 19/02; C12P 19/12; C12P 19/14

(52) U.S. Cl. CPC A23L 29/30; A23L 7/126; A23L 7/135; A23L 27/33; A23L 27/63; A23L 29/262; C12P 19/02; C12P 19/12; C12P 19/14; A23V 2002/00

(57) ABSTRACT

Methods of forming an ingredient for human consumption are provided herein. The methods may include isolating one or more soluble polysaccharides from a biomass, generating one or more oligosaccharides from the biomass, and combining the one or more isolated soluble polysaccharides with the generated oligosaccharides to form the ingredient. Methods of pretreating a biomass are also provided. The methods may include administering a physical pretreatment to a biomass, administering a gentle pretreatment to the physically pretreated biomass, and administering a strong pretreatment to the gently pretreated biomass. Ingredients for human consumption are also provided.

### CROSS REFERENCE

This application is a continuation of U.S. patent application Ser. No. 17/691,931 filed Mar. 10, 2022 (abandoned), which is a continuation of U.S. patent application Ser. No. 17/083,121, filed Oct. 28, 2020, now U.S. Pat. No. 11,297,865 issued Apr. 12, 2022, which is a continuation of International Patent Application PCT/EP2020/072929, filed Aug. 14, 2020, which this application claims the benefit of GB Patent Application No. 1911762.1, filed Aug. 16, 2019, GB Patent Application No. 1911764.7, filed Aug. 16, 2019, and GB Patent Application No. 2002315.6, filed Feb. 19, 2020, each of which is hereby incorporated by reference in its entirety.

### SEQUENCE LISTING

The instant application contains a Sequence Listing which has been submitted in ASCII format via EFS-Web and is hereby incorporated by in its entirety. Said ASCII copy, created Oct. 26, 2020, is named 56406_709_302_ST.txt and is 97.0 kilobytes in size

### BACKGROUND

Sugary foods and drinks are an important part of cultural and lifestyle habits across the world, but the sugar they contain has been linked to obesity, diabetes, poor dental health, and disruptive behavior in people. Because of this, consumer preferences have been shifting away from sugar-containing foods, and governments are increasingly implementing regulation to encourage the consumption of less sugar.

As such, industry has been searching for suitable low-calorie sweeteners for many decades to substitute for sugar in food and beverages. Unfortunately, many sugar substitutes are produced from non-natural resources, and often offer bitter undertones or other unpleasant tastes along with their sweetness, both of which consumers find unappealing. Moreover, while many sweeteners are able to mimic the sweetness of sugar in food and drinks, few are able to mimic the broad range of roles that sugar plays in food, such as adding bulk, modulating texture, providing structure, acting as a preservative, and modulating color and flavor through caramelization and Maillard reactions. In addition, many bulking sweeteners that are able to mimic these physical properties of sugar have gastrointestinal tolerance issues that limit their use to levels well below the amount required to replace sugar in a standard Western diet.

Dietary fiber is an important part of a positive diet and helps maintain digestive health and a well-regulated gut flora. Such fiber includes saccharides of varying chain lengths and types. In addition to being found naturally in a wide spectrum of foods, fiber can also be produced separately and added to other foods during their manufacture.

### SUMMARY

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In some embodiments, described herein are methods of producing ingredients for human consumption. A method of producing an ingredient for human consumption may comprise (a) isolating one or more soluble polysaccharides from a biomass; (b) contacting the remaining biomass with one or more enzymes to form one or more oligosaccharides; (c) isolating the one or more oligosaccharides; and (d) combining a portion of the one or more soluble polysaccharides from step (a) with a portion of the one or more oligosaccharides from step (c) to form the ingredient.

In some cases, the method may comprise purifying the isolated one or more soluble polysaccharides.

In some cases, the method may comprise purifying the isolated one or more oligosaccharides.

In some cases, the method may comprise treating the biomass to solubilize the one or more soluble polysaccharides.

In some cases, the method may comprise purifying the isolated one or more soluble polysaccharides.

In some cases, the treating comprises a thermochemical treatment.

In some cases, the thermochemical treatment comprises at least one of a hot water treatment or a hot alkali treatment.

In some cases, the hot alkali treatment uses an alkali with a pH of from 10 to 14.

In some cases, the hot alkali treatment uses at least one of sodium hydroxide, potassium hydroxide, sodium carbonate, calcium carbonate, calcium hydroxide, ammonium sulfate, ammonium hydroxide, or aqueous ammonia.

In some cases, the treating may be conducted at a temperature of from 30° C. to 180° C.

In some cases, the treating may be conducted for from 10 minutes to 24 hours.

In some cases, the one or more soluble polysaccharides and/or the one or more oligosaccharides are dried prior to step (d).

In some cases, the one or more soluble polysaccharides and/or the one or more oligosaccharides are dried subsequent to step (d).

In some cases, the ingredient may be soluble in water.

In some cases, the solubility of the ingredient in water may be at least 80 g of the ingredient per 100 g of water at 50° C.

In some cases, the method comprises combining the ingredient with a liquid to form a liquid ingredient.

In some cases, a viscosity of the liquid ingredient may be similar to a viscosity of corn syrup.

In some cases, a viscosity of the liquid ingredient may be similar to a viscosity of high-fructose corn syrup.

In some cases, the liquid ingredient has fewer calories per gram than corn syrup or high-fructose corn syrup.

In some cases, the liquid ingredient has a lower glycemic index than corn syrup or high-fructose corn syrup.

In some cases, the liquid comprises water.

In some cases, the liquid ingredient comprises at least 20% by dry weight of the at least one oligosaccharide and at least 2% by dry weight of the at least one polysaccharide.

In some cases, the liquid ingredient has a viscosity of from 5 cps to 100,000 cps, 8,000 cps to 100,000 cps, 10,000 cps to 50,000 cps, or 15,000 cps to 25,000 cps.

In some cases, the liquid ingredient comprises at least 2% by dry weight of xylan.

In some cases, the liquid ingredient comprises at least 2% by dry weight of mannan.

In some cases, the liquid ingredient comprises at least 2% by dry weight of a cellulose derivative.

In some cases, the liquid ingredient has a concentration of polysaccharides of from 0.1% to 50% w/v.

In some cases, the liquid ingredient comprises an amount of polysaccharide and oligosaccharide in a ratio from 1:100 to 1:1.

In some cases, the one or more soluble polysaccharides comprise at least one of a mannan, a xylan, a mixed-linkage glucan, a lignocellulose, a hemicellulose, a cellulose derivative, a chitosan, or a xyloglucan.

In some cases, the cellulose derivative comprises at least one of a cellulose acetate, a hydroxyethylcellulose, or a hydroxymethylcellulose.

In some cases, the biomass comprises at least one of a sugar cane biomass, a corn biomass, a wheat biomass, a hardwood biomass, or a softwood biomass.

In some cases, the one or more oligosaccharides comprise at least one of: i) a cello-oligosaccharide having a degree of polymerization (DP) of from two to six; ii) a xylo-oligosaccharide having a DP of from two to twelve; iii) an arabinoxylo-oligosaccharide having a DP of from three to fifteen; iv) a manno-oligosaccharide having a DP of from two to twelve; v) a mixed-linkage glucan oligosaccharide having a DP of from two to five; vi) a xyloglucan oligosaccharide having a DP of from four to twelve; or vii) a chito-oligosaccharide having a DP of from two to twelve.

In some cases, the ingredient comprises at least two of the oligosaccharides listed in (i) to (vii).

In some cases, the ingredient comprises the at least two oligosaccharides in a ratio from 1:9 to 1:1 in relation to each other.

In some embodiments, described herein are compositions for human consumption. The composition for human consumption may comprise: a soluble polysaccharide; and an oligosaccharide may comprise at least one of: (i) a cello-oligosaccharide having a degree of polymerization (DP) of from two to six; (ii) a xylo-oligosaccharide having a DP of from two to twelve; (iii) a manno-oligosaccharide having a DP of from two to twelve; (iv) an arabinoxylo-oligosaccharide having a DP of from three to fifteen; (v) a mixed-linkage glucan oligosaccharide having a DP of from two to five; or (vi) a chito-oligosaccharide having a DP of from two to twelve, wherein the composition comprises less than 5% by dry weight insoluble polysaccharides.

In some cases, the composition may be substantially free of insoluble polysaccharides.

In some cases, the composition may be soluble in water.

In some cases, the solubility of the composition in water may be at least 80 g of the composition per 100 g of water at 50° C.

In some cases, the composition further comprises a liquid, thereby forming a liquid ingredient.

In some cases, the liquid may be water.

In some cases, the liquid ingredient comprises at least 20% by dry weight of the at least one oligosaccharide and at least 2% by dry weight of the at least one polysaccharide.

In some cases, the liquid ingredient has a viscosity of from 5 cps to 100,000 cps, 8,000 cps to 100,000 cps, 10,000 cps to 50,000 cps, or 15,000 cps to 25,000 cps.

In some cases, the liquid ingredient comprises at least 2% by dry weight of xylan.

In some cases, the liquid ingredient comprises at least 2% by dry weight of mannan.

In some cases, the liquid ingredient comprises at least 2% by dry weight of a cellulose derivative.

In some cases, the liquid ingredient has a concentration of polysaccharides of from 0.1% to 50% w/v.

In some cases, the liquid ingredient comprises an amount of polysaccharide and oligosaccharide in a ratio from 1:100 to 1:1.

In some cases, the one or more soluble polysaccharides comprise at least one of a mannan, a xylan, a mixed-linkage glucan, a lignocellulose, a hemicellulose, a cellulose derivative, a chitosan, or a xyloglucan.

In some cases, the cellulose derivative comprises at least one of a cellulose acetate, a hydroxyethylcellulose, or a hydroxymethylcellulose.

In some cases, the biomass comprises at least one of a sugar cane biomass, a corn biomass, a wheat biomass, a hardwood biomass, or a softwood biomass.

In some cases, the one or more oligosaccharides comprise at least one of: i) a cello-oligosaccharide having a degree of polymerization (DP) of from two to six; ii) a xylo-oligosaccharide having a DP of from two to twelve; iii) an arabinoxylo-oligosaccharide having a DP of from three to fifteen; iv) a manno-oligosaccharide having a DP of from two to twelve; v) a mixed-linkage glucan oligosaccharide having a DP of from two to five; vi) a xyloglucan oligosaccharide having a DP of from four to twelve; or vii) a chito-oligosaccharide having a DP of from two to twelve.

In some cases, the composition comprises at least two of the oligosaccharides listed in (i) to (vii).

In some cases, the ingredient comprises the at least two oligosaccharides in a ratio from 1:9 to 1:1 in relation to each other.

In some embodiments, described herein are methods for producing ingredients for human consumption. The method for producing an ingredient for human consumption may comprise: (a) administering a physical pretreatment to a biomass to reduce an average size of the biomass; (b) administering a gentle pretreatment to the physically pretreated biomass, the gentle pretreatment may comprise: (i) incubating the physically pretreated biomass in an aqueous solution to solubilize monosaccharides and/or disaccharides from the physically pretreated biomass; and (ii) removing a portion of the solubilized monosaccharides and/or disaccharides from the aqueous solution; (c) administering a strong pretreatment to the gently pretreated biomass to increase the digestibility of the biomass; (d) contacting, in a solution or suspension, one or more polysaccharide-cleaving enzymes and the strongly pretreated biomass to form one or more oligosaccharides; and (e) enriching the solution or suspension to increase the concentration of the one or more oligosaccharides to form the ingredient.

In some cases, the gentle pretreatment may be an incubation cycle.

In some cases, the strong pretreatment may be a thermochemical treatment may comprise incubating the gently pretreated biomass in one of an acidic solution or an alkali solution.

In some cases, the method may further comprise removing at least 25% or 50% of the solubilized monosaccharides and/or disaccharides from the incubation solution at step (b) (ii).

In some cases, the strongly pretreated biomass composition after step (c) comprises less than 10% w/w monosaccharides.

In some cases, the method may further comprise purifying the one or more oligosaccharides from the solution or suspension.

In some cases, the strongly pretreated biomass composition after step (c) comprises less than 20% w/w monosaccharides.

In some cases, the method may further comprise repeating step (b).

In some cases, the step (b) may be conducted two, three, four, or five times.

In some cases, the method may further comprise repeating step (c).

In some cases, the step (c) may be conducted two, three, four, or five times.

In some cases, the method may further comprise concentrating the portion of the solubilized monosaccharides and/or disaccharides removed in step (b).

In some cases, the method may further comprise discarding the portion of the solubilized monosaccharides and/or disaccharides removed in step (b).

In some cases, the portion of the solubilized monosaccharides and/or disaccharides removed in step (b) may be not combined with the portion of the one or more oligosaccharides of step (e) to form the ingredient.

In some cases, the ingredient comprises less than 15% by dry weight monosaccharides.

In some cases, the ingredient comprises less than 50% by dry weight disaccharides.

In some cases, the ingredient may be substantially free of monosaccharides.

In some cases, the ingredient may be substantially free of disaccharides.

In some cases, the one or more oligosaccharides comprise at least one of: i) a cello-oligosaccharide having a degree of polymerization (DP) of from two to six; ii) a xylo-oligosaccharide having a DP of from two to twelve; iii) an arabinoxylo-oligosaccharide having a DP of from three to fifteen; iv) a manno-oligosaccharide having a DP of from two to twelve; v) a mixed-linkage glucan oligosaccharide having a DP of from two to five; vi) a xyloglucan oligosaccharide having a DP of from four to twelve; or vii) a chito-oligosaccharide having a DP of from two to twelve.

In some cases, the ingredient comprises at least two of the oligosaccharides listed in (i) to (vii).

In some cases, the ingredient comprises the at least two oligosaccharides in a ratio from 1:9 to 1:1 in relation to each other.

In some cases, the ingredient comprises at least one of sucrose, maltose, lactose, glucose, fructose, or galactose at less than 50% total dry w/w of the total dry w/w of all oligosaccharides of i-vii

In some cases, the monosaccharides and/or disaccharides comprise at least one of sucrose, maltose, lactose, glucose, fructose, or galactose.

In some cases, the step (b) solubilizes one or more organic acid in addition to the monosaccharides and/or disaccharides.

In some cases, the one or more organic acid comprises at least one of oxalate, tartrate, succinate, formate, citrate, malate, lactate or acetate.

In some cases, the total weight of oxalate, tartrate, succinate, formate, citrate, malate, lactate and acetate may be greater than 10% of the total weight of sucrose, maltose, lactose, glucose, fructose and galactose in the portion solubilized and removed in step b.

In some cases, the physical pretreatment of step (a) comprises at least one of chipping, chopping, milling, ball-milling, grinding, sprucing, or blending the biomass.

In some cases, the gentle pretreatment of step (b) occurs in an aqueous solution may comprise water.

In some cases, the gentle pretreatment of step (b) occurs at a temperature of from 5° C. to 150° C.

In some cases, the gentle pretreatment of step (b) may be conducted from 15 minutes to 1 hour.

In some cases, the strong pretreatment of step (c) comprises heating the gently pretreated biomass in the acidic solution or the alkali solution.

In some cases, the heating may be at a temperature of from 50° C. to 150° C.

In some cases, the heating may be conducted from 30 minutes to 4 hours.

In some cases, the step (c) comprises treating the gently pretreated biomass in an alkali solution having a pH from 8 to 11.

In some cases, the alkali solution comprises at least one of sodium hydroxide, potassium hydroxide, sodium carbonate, calcium carbonate, aqueous ammonia, ammonium sulfate, or ammonium hydroxide.

In some cases, the step (c) comprises treating the gently pretreated biomass in an acidic solution having a pH from 4 to 6.

In some cases, the acidic solution comprises at least one of sulfuric acid, hydrochloric acid, nitric acid, phosphoric acid, acetic acid, maleic acid, fumaric acid, or oxalic acid.

In some cases, the biomass comprises at least one of sugar cane, corn stover, corncob, wheat bran, wheat straw, hardwood, or softwood.

In some cases, the biomass comprises at least one of cellulose, chitin, chitosan, xylan, xyloglucan, mixed-linkage glucan, mannan, or lignocellulose.

In some cases, the one or more polysaccharide-cleaving enzymes comprises at least one of cellulase, xylanase, xyloglucanase, endo-glucanase, cellobiohydrolase, mannanase, lichenase, or lytic polysaccharide monooxygenase (LPMO).

In some cases, the one or more polysaccharide-cleaving enzymes comprises at least one of AA9, AA10, AA11, AA13, AA14, or AA15.

In some cases, the one or more of the polysaccharide-cleaving enzymes may be prepared from a filamentous fungi, such as Trichoderma reesei.

In some cases, the one or more polysaccharide-cleaving enzymes may be operably linked to a catalytic module.

In some cases, the one or more polysaccharide-cleaving enzymes may be operably linked to a non-catalytic module.

In some cases, the non-catalytic module may be a carbohydrate-binding module.

In some cases, a water-soluble composition for human consumption may comprise at least one of the following oligosaccharides: i) a cello-oligosaccharide having a degree of polymerization (DP) of from two to six; ii) a xylo-oligosaccharide having a DP of from two to twelve; iii) an arabinoxylo-oligosaccharide having a DP of from three to fifteen; iv) a manno-oligosaccharide having a DP of from two to twelve; v) a mixed-linkage glucan oligosaccharide having a DP of from two to five; vi) a xyloglucan oligosaccharide having a DP of from four to twelve; or vii) a chito-oligosaccharide having a DP of from two to twelve; and at least one of the following monosaccharides or disaccharides: sucrose, maltose, lactose, fructose, or galactose, wherein the total dry weight of the monosaccharides or disaccharides comprises less than 10% of the total dry weight of the oligosaccharides having a DP of from two to twelve.

In some cases, the ingredient may comprise at least two of the oligosaccharides listed in (i) to (vii). In some cases, the ingredient may further comprise at least one of the following organic acids: oxalate, tartrate, succinate, formate, citrate, malate, lactate, or acetate. In some cases, the ingredient may comprise at least two of the organic acids. In some cases, the ingredient may comprise at least two of the oligosaccharides listed in (i) to (vii) in a ratio from 1:9 to 1:1 in relation to each other.

In some cases, a composition may comprise at least one monosaccharide or disaccharide selected from the group consisting of: glucose, fructose, or sucrose; at least one organic acid selected from the group consisting of oxalate, tartrate, succinate, formate, citrate, malate, lactate, or acetate, wherein the total weight of the organic acids is greater than 10% of the total weight of the monosaccharides or disaccharides.

In some cases, a method for producing an ingredient for human consumption may comprise: (a) administering a physical pretreatment to a biomass to reduce an average size of the biomass; (b) administering a gentle pretreatment to the physically pretreated biomass, the gentle pretreatment comprising: (i) incubating the physically pretreated biomass in a water solution to solubilize monosaccharides and/or disaccharides from the physically pretreated biomass; and (ii) removing a portion of the solubilized monosaccharides and/or disaccharides from the water solution; (c) administering a strong pretreatment to the gently pretreated biomass to solubilize polysaccharides and to increase the digestibility of the plant biomass; (d) isolating one or more solubilized polysaccharides from the biomass; (e) contacting the remaining biomass with one or more enzymes to form one or more oligosaccharides; (f) isolating the one or more oligosaccharides; and (g) combining a portion of the one or more soluble polysaccharides from step (d) with a portion of the one or more oligosaccharides from step (f) to form the ingredient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 depicts the results of a thin-layer chromatography (TLC) analysis showing the monosaccharide and disaccharide content of the indicated biomasses after one to five washing cycles (incubation pretreatments).

FIG. 2 depicts the results of a TLC analysis comparing the levels of monosaccharides and disaccharides in an enzyme hydrolysate product mixture when washing (incubation pretreatment) has (+) and has not (−) been performed.

FIG. 3 depicts the results of making a glaze with compositions of the present disclosure and a comparative composition.

FIG. 4 depicts baked products using a liquid ingredient of the present disclosure.

FIG. 5 depicts a high-performance anion exchange chromatography (HPAEC) analysis of Sample 4.

FIG. 6 depicts cereal bars produced using a liquid ingredient of the present disclosure.

FIG. 7 depicts an HPAEC chromatogram of the saccharides in the water post-washing of corncobs.

FIG. 8 is a simplified flow diagram depicting a method of treating biomass according to some embodiments of the present disclosure.

FIG. 9 is a simplified flow diagram depicting another method of treating biomass according to some embodiments of the present disclosure.

FIG. 10A is a diagram illustrating methods of processing biomass.

FIG. 10B depicts measurement of saccharides in multiple samples.

FIG. 10C depicts measurement of organic acids in multiple samples.

FIG. 10D depicts and visual observation of the samples processed according to the methods of FIG. 10A.

FIG. 11A illustrates a comparison between a cereal bar made using soluble polysaccharides and a cereal bar made using insoluble polysaccharides.

FIG. 11B depicts the hardness and stickiness of the cereal bars of FIG. 11A using a penetration test.

FIG. 11C depicts the hardness of the cereal bars of FIG. 11A using a cutting test.

### DETAILED DESCRIPTION

### Introduction

Provided herein are methods of forming one or more ingredients. The methods may include isolating one or more soluble polysaccharides from a biomass or feedstock (e.g., a plant biomass). The remaining biomass may then be contacted or treated with one or more enzymes to form one or more oligosaccharides and the one or more oligosaccharides may be enriched or isolated. Furthermore, at least a portion of the one or more soluble polysaccharides isolated from the biomass may be combined with a portion of the one or more enriched or isolated oligosaccharides to form the ingredient.

The texture of the ingredient may be consistent and smooth. Furthermore, the ingredient may have certain properties such that the ingredient may be used as a sweetener and/or a sugar substitute. Properties of the ingredient can include sweetness, smooth texture, desirable mouthfeel, ability to bind, ability to glaze or form a glaze, moistness, viscosity, ability to bulk, and/or ability to caramelize. In comparison to corn syrup or high-fructose corn syrup, the ingredient can also have fewer calories, reduced glycemic index, reduced glycemic load, increased fiber, and/or reduced sugar.

Also provided herein are methods for producing an ingredient for incorporation into a foodstuff, a nutraceutical, and/or a cosmetic, wherein the methods may include one or more pretreatment steps performed on a biomass. For example, the method may include a first pretreatment step, a second pretreatment step, a third pretreatment step, or additional pretreatment steps performed on a biomass. The pretreatment steps may be performed in a specified order.

The biomass used to produce one or more oligosaccharides may be a plant biomass. Examples of plant biomass include, but are not limited to, sugar cane, corn stover, corncob, wheat bran, wheat straw, hardwood, or softwood. In some cases, the biomass may comprise cellulose, chitin, chitosan, xylan, xyloglucan, mixed-linkage glucan, mannan, or lignocellulose.

The biomass may be digested into one or more oligosaccharides. The biomass digestion may be performed enzymatically. The enzymatic digestion may be performed after one or more pretreatment steps. The one or more pretreatment steps may be performed to reduce the size of a biomass and/or increase the surface area of the biomass available for digestion. The one or more pretreatment steps may include one or more washing steps, solubilizing steps, or pre-digestion treatments. In some cases, one or more pretreatment steps may be performed to reduce the monosaccharides and/or disaccharides present in the biomass. In some cases, one or more pretreatment steps may be performed to recover a soluble polysaccharide fraction from the biomass.

During the one or more pretreatment steps, monosaccharides and/or disaccharides may be removed from a starting material (e.g., a biomass). Stated another way, monosaccharides and/or disaccharides may be removed from the biomass during one or more of the pretreatment steps. Accordingly, no, or substantially no, monosaccharides and/or disaccharides may be yielded upon completion of the one or more pretreatment steps. That is, the pretreated biomass may comprise no or substantially no monosaccharides and/or disaccharides. This can improve the efficiency of the method for producing the ingredient for incorporation into a foodstuff, a nutraceutical, and/or a cosmetic as provided herein. For example, as a portion of the monosaccharides and/or disaccharides has already been removed from the biomass during the one or more pretreatment steps, less purification of the ingredient (e.g., to remove monosaccharides and/or disaccharides) may be needed. Specifically, fewer filtration steps, or less stringent filtration steps, may be needed during purification to generate the ingredient as disclosed herein.

A first pretreatment step (pretreatment step 1) may include physically treating a biomass (e.g., chipping the biomass). A second pretreatment step (pretreatment step 2 or gentle pretreatment) may include subjecting the physically treated biomass to an incubation cycle or a washing cycle. The incubation cycle may include incubating the physically treated biomass (from pretreatment step 1) in an aqueous solution to solubilize monosaccharides and/or disaccharides from the physically treated biomass. The incubation cycle may also include removing a portion of the solubilized monosaccharides and/or disaccharides from the aqueous solution. In some cases, the aqueous solution may include water. In some other cases, the incubation cycle may be performed at about 25° C. for a period of about 30 minutes to about 1.5 hours. Pretreatment step 2 may be a gentle pretreatment step. For example, the conditions (e.g., solution, temperature, time, etc.) of pretreatment step 2 may be gentler than the conditions of pretreatment step 3 as described in further detail below.

A third pretreatment step (pretreatment step 3 or strong pretreatment) may include treating the incubated biomass from pretreatment step 2 in one of an acidic solution or an alkali solution. Pretreatment step 3 can improve the digestibility of the biomass (e.g., by an enzyme). Pretreatment step 3 may also improve enzyme access to the biomass. In various instances, pretreatment step 3 may occur in an alkali solution (e.g., a 1% w/v NaOH solution) at a temperature above room temperature (e.g., at from about 90° C. to about 110° C.). Furthermore, pretreatment step 3 may be conducted by being held at the desired temperature for about 30 minutes to 1 hour. For instance, the effective temperature, in this example 90° C., is held for an hour (this can be altered depending on the desired characteristic). The solution in the third pretreatment step can be retained. Stated another way, the solution may not be discarded as the treated biomass moves from the pretreatment steps to the steps after the pretreatment steps as described in further detail below. In some cases, pretreatment step 3 may include a thermochemical treatment. That is, pretreatment step 3 may be performed in an acidic or alkali solution and/or pretreatment step 3 may be performed at a temperature above room temperature.

In various instances, after the one or more pretreatment steps, the method for producing an ingredient for human consumption may include contacting, in a solution or suspension, one or more polysaccharide-cleaving enzymes and the biomass from pretreatment step 3 to form one or more oligosaccharides. The method may further include enriching the solution or suspension to increase the concentration of the one or more oligosaccharides to form the ingredient (e.g., the ingredient for human consumption).

The pretreatment steps may increase the efficiency of the method in comparison to some other methods. The efficiency may be improved insofar as less extensive downstream processing may be needed. In some cases, downstream processing may include the process of removing monosaccharides and/or disaccharides from the pretreated biomass. In some embodiments, it may be difficult to remove disaccharides alone, for example, from an intermediate solution or fraction that is generated by the enzyme digestion. Thus, it may be more efficient to remove the disaccharides during the pretreatment steps.

The steps of the downstream processing can include ion-exchange chromatography, ultrafiltration, microfiltration, nanofiltration, etc. Part of the role of the nanofiltration step may be to remove excess monosaccharides from the oligosaccharide mixture. This nanofiltration may be performed multiple times to arrive at desirable monosaccharide levels. The number of such nanofiltration steps may be reduced when there are fewer monosaccharides in the pretreated biomass (e.g., when the monosaccharides have been removed by washing or incubation). Furthermore, ultrafiltration generally cannot differentiate between desirable disaccharides (e.g., cellobiose) and undesirable disaccharides. Accordingly, removing the undesirable disaccharides during pretreatment and generating the desirable disaccharides during the enzyme treatment step can also reduce the number of steps involved or needed during downstream processing.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a sample” includes a plurality of samples, including mixtures thereof.

The term “about,” as used herein, can mean within 1 or more than 1 standard deviation. Alternatively, about can mean a range of up to 10%, up to 5%, or up to 1% of a given value. For example, about can mean up to ±10%, ±9%, ±8%, ±7%, ±6%, ±5%, ±4%, ±3%, ±2%, or ±1% of a given value.

As used herein, “food” and “foodstuff” generally refer to any item destined for consumption, which may be consumption by a human or by any other animal. It may be food, feed, beverage, or an ingredient to be used in the production of any of the above.

As used herein, “nutraceutical” generally refers to any composition introduced into a human or other animal, whether by ingestion, injection, absorption, or any other method, for the purpose of providing nutrition to the human or other animal. Use of such a nutraceutical may take the form of a drink with added dietary fiber, a prebiotic additive, a pill or other capsule, or any other suitable use.

As used herein, “cosmetic” generally refers to any composition which is intended for use on humans or other animals to increase their aesthetic appeal or prevent future loss of aesthetic appeal, as well as any other compositions known in general parlance as cosmetics. Aesthetic appeal is not limited to visual aesthetics but applies as well to textural or any other appeal. The cosmetic may be mascara, foundation, lip gloss, eyeshadow, eyeliner, primer, lipstick, blush, nail polish, bronzer, or any other makeup; shampoo, conditioner, styling mousse, styling gel, hairspray, hair dye, hair wax, or any other hair product; moisturizer, exfoliant, sun cream, cleanser, toothpaste, cream, lotion, ointment, or any other composition effective in modifying teeth, skin, hair, or other parts of the body in some aesthetic way. Further, the cosmetic may be a composition used as a component of a face mask, brush, hair roller, other styling device, other solid structure, or any other suitable composition.

As used herein, “ingredient” generally refers to any composition suitable for incorporation into a foodstuff, cosmetic, or nutraceutical product, which may include those which are used directly as the product itself. It may be a dry or liquid ingredient, unless it is specifically referred to as “dry” or “liquid.” This includes compositions that may be deemed to be an intermediate during a method of the disclosure, such as a composition formed after the combining of the one or more oligosaccharides and the one or more soluble polysaccharides prior to any further purification, optimization, drying, dissolving, or any other such steps, as well as including the final composition obtained from the method.

As used herein, “polysaccharide” generally refers to a saccharide polymer of any length greater than about 20 residues. Polysaccharides may be highly branched, lightly branched, or unbranched. Polysaccharides may include any manner of glycosidic bond in any combination; any number of, for example, α or β linkages; and any combination of monomer types, such as glucose, glucosamine, mannose, xylose, galactose, fucose, fructose, glucuronic acid, arabinose, or derivatives thereof, such as any combination of the above monomers decorated with acetyl or other groups. The polysaccharide may be a cellulosic or hemicellulosic polymer. Hemicellulosic polymers envisaged include xylan, glucuronoxylan, arabinoxylan, glucomannan, and xyloglucan. In some embodiments, the cellulosic polymer may be cellulose.

As used herein, “lignocellulose” generally refers to polysaccharide-comprising aggregates that are, or are derived from, plant cell wall material. For example, they may include one or more of the following polysaccharides associated together: cellulose, xylan, mannan, and mixed-linkage glucan.

As used herein “highly branched,” “lightly branched,” and “unbranched” generally refer to the number of side-chains per stretch of main chain in a saccharide. Highly branched saccharides have on average from 4 to 10 side chains per 10 main-chain residues, slightly branched saccharides have on average from 1 to 3 side chains per 10 main-chain residues, and unbranched saccharides have only one main chain and no side chains. The average is calculated by dividing the number of side chains in a saccharide by the number of main-chain residues.

As used herein, “saccharide” generally refers to any polysaccharide and/or oligosaccharide, such as a monosaccharide and/or a disaccharide.

As used herein, “oligosaccharide” generally refers to saccharide polymers having chain lengths less than or equal to about 20 saccharide residues. Oligosaccharides may be highly branched, lightly branched, or unbranched; and may include glycosidic bonds in any combination, any number of α or β linkages, and any combination of monomer types, such as glucose, glucosamine, mannose, xylose, galactose, fucose, fructose, glucuronic acid, arabinose, or derivatives thereof. Suitable derivatives include the above monomers including acetyl or other groups.

As used herein, “monosaccharide” and “disaccharide” generally refer to saccharide compounds consisting of one or two residues, respectively. Monosaccharides are compounds such as glucose, glucosamine, xylose, galactose, fucose, fructose, glucuronic acid, arabinose, galacturonic acid, or epimers or other derivatives thereof. Suitable derivatives include acetyl or other groups. Disaccharides are compounds consisting of two monosaccharides joined via any glycosidic bond.

As used herein, “cello-oligosaccharides” generally refer to oligosaccharides composed of one or more glucose residues linked by β-1,4-glycosidic bonds, and may be chemically related to that by oxidation, reduction, esterification, epimerization, or another chemical modification.

As used herein, “xylo-oligosaccharides” generally refer to oligosaccharides composed primarily of xylose residues (typically linked by β-1,4-glycosidic bonds) and may also contain glucuronic acid residues and/or arabinose residues and/or acetyl groups and/or any other modification, and may be chemically related to that by oxidation, reduction, esterification, epimerization, further glycosylation, or another chemical modification.

As used herein, “arabinoxylo-oligosaccharides” generally refer to oligosaccharides composed of xylose residues (typically linked by β-(1→4)-bonds substituted with arabinose side-chains), typically linked by (1→2)-bonds or (1→3)-bonds) and may be chemically related to that by oxidation, reduction, esterification, epimerization, further glycosylation, or another chemical modification.

As used herein, “mixed-linkage glucan-oligosaccharides” generally refer to oligosaccharides composed of one or more glucose residues linked by at least one β-1,3-glycosidic bond and at least one β-1,4-glycosidic bond, and may be chemically related to that by oxidation, reduction, esterification, epimerization, or another chemical modification

As used herein, “manno-oligosaccharides” generally refer to oligosaccharides composed of one or more mannose residues and optionally containing one or more glucose and/or galactose residues, and may be chemically related to that by oxidation, reduction, esterification, epimerization, or another chemical modification.

As used herein, “chito-oligosaccharides” generally refer to oligosaccharides composed of one or more glucosamine and/or N-acetyl-glucosamine residues, and may be chemically related to that by oxidation, reduction, esterification, epimerization, or another chemical modification.

As used herein, “cellulose” generally refers to polysaccharides composed of glucose residues linked by β-1,4-glycosidic bonds, and derivatives thereof. As used herein, “xylan” generally refers to polysaccharides composed of a backbone of xylose residues and may also contain glucuronic acid residues and/or arabinose residues and/or acetyl groups and/or any other modification. As used herein, “mixed-linkage glucan” generally refers to polysaccharides composed of glucose residues linked by β-1,3-glycosidic bonds and β-1,4-glycosidic bonds. As used herein, “mannan” generally refers to polysaccharides composed of greater than 40% mannose residues and optionally containing glucose and/or galactose residues. As used herein, “chitin” or “chitosan” generally refer to polysaccharides composed of glucosamine and/or N-acetyl-glucosamine residues. The polysaccharides of cellulose, xylan, mixed-linkage glucan, mannan, chitin, or chitosan may include chemical variants that have been modified by oxidation, reduction, esterification, epimerization, or another chemical modification.

As used herein, “soluble,” “solubility,” and grammatical variants thereof generally refer to solubility in an aqueous solution (e.g., water). As used herein, “solubilize” generally refers to a solid becoming incorporated into an aqueous solution or a liquid so as to form a solution.

As used herein, “suspension” generally refers to a composition comprising at least two immiscible phases, for example, a solid phase and a liquid phase, wherein the weight of the solid phase may be, as a percentage of the weight of the composition, in the range of from about 0.5% to about 30%, about 1% to about 20%, about 2% to about 15%, or about 3% to about 10%. The suspension may comprise a suitable solvent, which may be water.

As used herein, “viscosity” generally refers to a quantity expressing the magnitude of internal friction in a fluid, as measured by the force per unit area resisting uniform flow. The viscosity can be measured by a variety of methods, but the values given herein, unless indicated otherwise, refer to those obtained by using a Brookfield HDB VE roto-viscometer using standard testing procedures, operated as per the manufacturer's instructions with respect to ranges, and with a 400 mL sample taken in a tall-form beaker to ensure that no container effects occur.

As used herein, “dissolved” generally refers to a solid becoming incorporated into a liquid so as to form a solution.

### I. Pretreatment

### Physical Pretreatment

A mechanical or physical pretreatment may be performed on a biomass for the digestion of the biomass into one or more oligosaccharides. The mechanical and/or physical pretreatment may be the first pretreatment step in the process of biomass digestion. Alternatively, the mechanical or physical pretreatment step may be performed after another pretreatment step. For instance, a mechanical or physical pretreatment step may be performed after another pretreatment step, such as a washing pretreatment step.

A biomass may be mechanically or physically pretreated to reduce the size of the biomass. Examples of mechanical or physical pretreatment steps include, but are not limited to: chipping, chopping, milling, ball-milling, grinding, sprucing, blending, and/or steam explosion of the biomass. More than one physical pretreatment may be performed on the biomass.

### Solubilizing Step

The biomass may undergo a solubilizing step. The solubilizing step may be, or be a portion of, a gentle pretreatment step, which solubilizes a polysaccharide fraction or a monosaccharide and/or disaccharide fraction from the biomass. The solubilizing pretreatment step may be a washing step, an incubation step, a thermochemical step, or a chemical treatment step. The solubilizing pretreatment may be performed before a physical or mechanical pretreatment step. The solubilizing pretreatment may be performed after a physical or mechanical pretreatment step.

In some embodiments, the solubilizing step may be performed to remove a fraction of soluble polysaccharides. Soluble polysaccharides can be added to one or more oligosaccharides, for example, upon purification of the soluble polysaccharides. The soluble polysaccharide fraction may be used to produce food types of desired taste, texture, quality, adhesiveness, and smell. Presence of solubilized polysaccharides may also help produce a better-quality product which does not produce sediments (or graininess) in a food product.

The solubilizing step may be a chemical or thermochemical treatment of the biomass. The chemical or thermochemical treatment may comprise one or more aqueous solutions. The aqueous solution may comprise one or more salts, acids, alkalis, or ions. The aqueous solution may comprise one or more of sodium hydroxide, potassium hydroxide, sodium carbonate, calcium carbonate, calcium hydroxide, ammonium sulfate, ammonium hydroxide, aqueous ammonia, dilute sulfuric acid, dilute acetic acid, dilute hydrochloric acid, or dilute phosphoric acid.

The aqueous solution may be an alkali solution with a pH from 10 to 14. The aqueous solution may be an alkali solution with a pH from 10 to 11, 10 to 12, 10 to 13, 10 to 14, 11 to 12, 11 to 13, 11 to 14, 12 to 13, 12 to 14, or 13 to 14. The aqueous solution may be an acid solution with a pH from 2 to 6. The aqueous solution may be an acid solution with a pH from 2 to 3, 2 to 4, 2 to 5, 2 to 6, 3 to 4, 3 to 5, 3 to 6, 4 to 5, 4 to 6, or 5 to 6.

The solubilization step may be performed at a temperature from 30° C. to 180° C. The solubilization step may be performed at a temperature of at least 30° C. The solubilization step may be performed at a temperature of at most 180° C. The solubilization step may be performed at a temperature from 30° C. to 60° C., 30° C. to 90° C., 30° C. to 120° C., 30° C. to 150° C., 30° C. to 180° C., 60° C. to 90° C., 60° C. to 120° C., 60° C. to 150° C., 60° C. to 180° C., 90° C. to 120° C., 90° C. to 150° C., 90° C. to 180° C., 120° C. to 150° C., 120° C. to 180° C., or 150° C. to 180° C. The solubilization step may be performed at a temperature of at least 30° C., 60° C., 90° C., 120° C., 150° C., or 180° C.

The duration of the solubilizing step may be altered according to the biomass being used, the soluble polysaccharide component desired, and/or the complexity of the soluble polysaccharide desired. The solubilizing step may be performed from about 10 minutes to about 24 hours. The solubilization step may be performed for at least 10 minutes. The solubilization step may be performed for at most 60 minutes. The solubilization step may be performed from 10 minutes to 30 minutes, 10 minutes to 60 minutes, or 30 minutes to 60 minutes. The solubilization step may be performed for at least 10 minutes, 30 minutes, or 60 minutes. The solubilization step may be performed for 1 hour to 24 hours. The solubilization step may be performed for at least 1 hour. The solubilization step may be performed for at most 24 hours. The solubilization step may be performed for 1 hour to 4 hours, 1 hour to 8 hours, 1 hour to 12 hours, 1 hour to 16 hours, 1 hour to 20 hours, 1 hour to 24 hours, 4 hours to 8 hours, 4 hours to 12 hours, 4 hours to 16 hours, 4 hours to 20 hours, 4 hours to 24 hours, 8 hours to 12 hours, 8 hours to 16 hours, 8 hours to 20 hours, 8 hours to 24 hours, 12 hours to 16 hours, 12 hours to 20 hours, 12 hours to 24 hours, 16 hours to 20 hours, 16 hours to 24 hours, or 20 hours to 24 hours. The solubilization step may be performed for at least 1 hour, 4 hours, 8 hours, 12 hours, 16 hours, 20 hours, or 24 hours.

Soluble polysaccharides may be removed from the solution after a solubilizing step. A fraction of the solubilized polysaccharides may be removed from the solubilized fraction. In some cases, at least 5% of the solubilized polysaccharides may be removed and/or purified. In some cases, at most 100% of the solubilized polysaccharides may be removed and/or purified. In some cases, 5% to 10%, 5% to 20%, 5% to 30%, 5% to 40%, 5% to 60%, 5% to 80%, 5% to 90%, 5% to 100%, 10% to 20%, 10% to 30%, 10% to 40%, 10% to 60%, 10% to 80%, 10% to 90%, 10% to 100%, 20% to 30%, 20% to 40%, 20% to 60%, 20% to 80%, 20% to 100%, 30% to 40%, 30% to 60%, 30% to 80%, 30% to 100%, 40% to 60%, 40% to 80%, 40% to 100%, 60% to 80%, or 60% to 100% of the solubilized polysaccharides may be removed and purified. In some cases, at least about 5%, 10%, 20%, 30%, 40%, 60%, 80%, or 100% of the solubilized polysaccharides may be removed and purified.

### Gentle Pretreatment

The biomass may undergo a gentle pretreatment step. The gentle pretreatment step may solubilize a polysaccharide fraction or a monosaccharide and/or disaccharide fraction from the biomass. In some cases, the gentle pretreatment step may include at least a portion, or all, of the solubilizing step. Stated another way, the solubilizing step may be a portion or sub-step of the gentle pretreatment step. The gentle pretreatment step may be a washing step, an incubation step, a thermochemical step, or a chemical treatment step. The gentle pretreatment may be performed before a physical or mechanical pretreatment step. The gentle pretreatment may be performed after a physical or mechanical pretreatment step. The gentle pretreatment may be performed simultaneously with a physical or mechanical pretreatment step. The gentle pretreatment step may be performed one or more times. The gentle pretreatment may be performed 2, 3, 4, 5, 6, 7, 8, 9, or 10 times.

The gentle pretreatment step may be an incubation step or a washing step. The biomass (physically treated or untreated) may be incubated in an aqueous solution. The aqueous solution may be water, or it may comprise salts, acids, alkali, ions, alcohols, and/or other chemicals. The pH of the aqueous solution may be from 6.2 to 8.5. The pH of the aqueous solution may be at least 6.2. The pH of the aqueous solution may be at most 8.5. The pH of the aqueous solution may be from 6.2 to 6.5, 6.2 to 7, 6.2 to 7.2, 6.2 to 7.5, 6.2 to 7.7, 6.2 to 8, 6.2 to 8.2, 6.2 to 8.5, 6.5 to 7, 6.5 to 7.2, 6.5 to 7.5, 6.5 to 7.7, 6.5 to 8, 6.5 to 8.2, 6.5 to 8.5, 7 to 7.2, 7 to 7.5, 7 to 7.7, 7 to 8, 7 to 8.2, 7 to 8.5, 7.2 to 7.5, 7.2 to 7.7, 7.2 to 8, 7.2 to 8.2, 7.2 to 8.5, 7.5 to 7.7, 7.5 to 8, 7.5 to 8.2, 7.5 to 8.5, 7.7 to 8, 7.7 to 8.2, 7.7 to 8.5, 8 to 8.2, 8 to 8.5, or 8.2 to 8.5. The pH of the aqueous solution may be from 6.2, 6.5, 7, 7.2, 7.5, 7.7, 8, 8.2, or 8.5.

The pH of the aqueous solution may be from 9 to 12. The pH of the aqueous solution may be from at least 9. The pH of the aqueous solution may be from at most 12. The pH of the aqueous solution may be from 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. The pH of the aqueous solution may be from 9, 9.5, 10, 10.5, 11, 11.5, or 12.

The incubation step may be performed for 15 minutes to 60 minutes. The incubation step may be performed for at least 15 minutes. The incubation step may be performed for at most 60 minutes. The incubation step may be performed for 15 minutes to 30 minutes, 15 minutes to 45 minutes, 15 minutes to 60 minutes, 30 minutes to 45 minutes, 30 minutes to 60 minutes, or 45 minutes to 60 minutes. The incubation step may be performed for at least 15 minutes, 30 minutes, 45 minutes, or 60 minutes. The incubation step may be performed for 1 hour to 24 hours. The incubation step may be performed for at least 1 hour. The incubation step may be performed for at most 24 hours. The incubation step may be performed for 1 hour to 4 hours, 1 hour to 8 hours, 1 hour to 12 hours, 1 hour to 16 hours, 1 hour to 20 hours, 1 hour to 24 hours, 4 hours to 8 hours, 4 hours to 12 hours, 4 hours to 16 hours, 4 hours to 20 hours, 4 hours to 24 hours, 8 hours to 12 hours, 8 hours to 16 hours, 8 hours to 20 hours, 8 hours to 24 hours, 12 hours to 16 hours, 12 hours to 20 hours, 12 hours to 24 hours, 16 hours to 20 hours, 16 hours to 24 hours, or 20 hours to 24 hours. The incubation step may be performed for at least 1 hour, 4 hours, 8 hours, 12 hours, 16 hours, 20 hours, or 24 hours.

Solubilized polysaccharides, monosaccharides, and/or disaccharides may be removed from the aqueous solution after the gentle pretreatment step. In some cases, monosaccharides and/or disaccharides may be removed from the aqueous solution after the gentle pretreatment step.

Soluble polysaccharides may be removed from the solution after a gentle pretreatment step. At least a portion of the solubilized polysaccharides may be removed from the solubilized fraction. In some cases, at least 5% of the solubilized polysaccharides may be removed and/or purified. In some cases, at most 100% of the solubilized polysaccharides may be removed and/or purified. In some cases, 5% to 10%, 5% to 20%, 5% to 30%, 5% to 40%, 5% to 60%, 5% to 80%, 5% to 100%, 10% to 20%, 10% to 30%, 10% to 40%, 10% to 60%, 10% to 80%, 10% to 100%, 20% to 30%, 20% to 40%, 20% to 60%, 20% to 80%, 20% to 100%, 30% to 40%, 30% to 60%, 30% to 80%, 30% to 100%, 40% to 60%, 40% to 80%, 40% to 100%, 60% to 80%, or 60% to 100% of the solubilized polysaccharides may be removed and/or purified. In some cases, at least about 5%, 10%, 20%, 30%, 40%, 60%, 80%, or 100% of the solubilized polysaccharides may be removed and/or purified after the gentle pretreatment step.

Monosaccharides and/or disaccharides may be removed from the solution after a gentle pretreatment step. A fraction of the solubilized monosaccharides and/or disaccharides may be removed from the aqueous solution after the incubation step. In some cases, at least 5% of the solubilized monosaccharides and/or disaccharides may be removed. In some cases, at most 100% of the solubilized monosaccharides and/or disaccharides may be removed. In some cases, 5% to 10%, 5% to 20%, 5% to 30%, 5% to 40%, 5% to 60%, 5% to 80%, 5% to 100%, 10% to 20%, 10% to 30%, 10% to 40%, 10% to 60%, 10% to 80%, 10% to 100%, 20% to 30%, 20% to 40%, 20% to 60%, 20% to 80%, 20% to 100%, 30% to 40%, 30% to 60%, 30% to 80%, 30% to 100%, 40% to 60%, 40% to 80%, 40% to 100%, or 60% to 100% of the solubilized monosaccharides and/or disaccharides may be removed. In some cases, at least about 5%, 10%, 20%, 30%, 40%, 60%, 80%, or 100% of the solubilized monosaccharides and/or disaccharides may be removed and/or purified after the gentle pretreatment step. The monosaccharide and/or disaccharide portion may be discarded after the incubation step. In certain instances, the portion of the solubilized monosaccharides and/or disaccharides removed in this step may not be combined with the portion of the one or more oligosaccharides produced in the biomass treatment.

The aqueous solution may be removed from the biomass after the gentle pretreatment step. A portion of the aqueous solution may be removed from the biomass after the gentle pretreatment step. 5% to 100% of the aqueous solution may be removed from the biomass after the gentle pretreatment step. At least 5% of the aqueous solution may be removed from the biomass after the gentle pretreatment step. At most 98% of the aqueous solution may be removed from the biomass after the gentle pretreatment step. Five percent to 10%, 5% to 20%, 5% to 40%, 5% to 50%, 5% to 60%, 5% to 80%, 5% to 90%, 5% to 98%, 10% to 20%, 10% to 40%, 10% to 50%, 10% to 60%, 10% to 80%, 10% to 90%, 10% to 98%, 20% to 40%, 20% to 50%, 20% to 60%, 20% to 80%, 20% to 90%, 20% to 98%, 40% to 50%, 40% to 60%, 40% to 80%, 40% to 90%, 40% to 98%, 50% to 60%, 50% to 80%, 50% to 90%, 50% to 98%, 60% to 80%, 60% to 90%, 60% to 98%, 80% to 90%, 80% to 98%, or 90% to 98% of the aqueous solution may be removed from the biomass after the gentle pretreatment step. At least 5%, 10%, 20%, 40%, 50%, 60%, 80%, 90%, or 98% of the aqueous solution may be removed from the biomass after the gentle pretreatment step. A portion of the aqueous solution may be removed using a filter press, centrifugation, sedimentation, filtration, and/or any other suitable method.

### Strong Pretreatment

The biomass may undergo a strong pretreatment step. The strong pretreatment step may solubilize a polysaccharide fraction or a monosaccharide and/or disaccharide fraction from the biomass. The strong pretreatment step may be performed to make the biomass more digestible by enzymes. The strong pretreatment may help disrupt the hydrogen bonds in the biomass. The strong pretreatment step may be a washing step, a thermochemical step, or a chemical treatment step. The strong pretreatment may be performed before a physical or mechanical pretreatment step. The strong pretreatment step may be performed after a physical or mechanical pretreatment step. The strong pretreatment step may be performed before a gentle pretreatment step. The strong pretreatment step may be performed after a gentle pretreatment step. The strong pretreatment step may be performed one or more times. The strong pretreatment may be performed 2, 3, 4, 5, 6, 7, 8, 9, or 10 times.

The strong pretreatment step may be a thermochemical treatment. The chemical or thermochemical treatment may comprise one or more aqueous solutions. The aqueous solution may comprise one or more salts, acids, alkalis, or ions. The aqueous solution may be an alkali solution comprising one or more of sodium hydroxide, potassium hydroxide, sodium carbonate, calcium carbonate, calcium hydroxide, ammonium sulfate, ammonium hydroxide, or aqueous ammonia. The aqueous solution may be an acidic solution comprising at least one of sulfuric acid, hydrochloric acid, nitric acid, phosphoric acid, acetic acid, maleic acid, fumaric acid, or oxalic acid. In some embodiments, upon completion of the one or more strong pretreatment steps, the aqueous solution may be retained. In other words, the aqueous solution may not be discarded or rejected.

The thermochemical pretreatment may be performed at a pH of from about 2 to 6.5. The thermochemical pretreatment may be performed at a pH of at least 2. The thermochemical pretreatment may be performed at a pH of at most 6.5. The thermochemical pretreatment may be performed at a pH of 2 to 2.5, 2 to 3, 2 to 3.5, 2 to 4, 2 to 4.5, 2 to 5, 2 to 5.5, 2 to 6, 2 to 6.5, 2.5 to 3, 2.5 to 3.5, 2.5 to 4, 2.5 to 4.5, 2.5 to 5, 2.5 to 5.5, 2.5 to 6, 2.5 to 6.5, 3 to 3.5, 3 to 4, 3 to 4.5, 3 to 5, 3 to 5.5, 3 to 6, 3 to 6.5, 3.5 to 4, 3.5 to 4.5, 3.5 to 5, 3.5 to 5.5, 3.5 to 6, 3.5 to 6.5, 4 to 4.5, 4 to 5, 4 to 5.5, 4 to 6, 4 to 6.5, 4.5 to 5, 4.5 to 5.5, 4.5 to 6, 4.5 to 6.5, 5 to 5.5, 5 to 6, 5 to 6.5, 5.5 to 6, 5.5 to 6.5, or 6 to 6.5. The thermochemical pretreatment may be performed at a pH of about 2, 2.5, 3, 3.5, 4, 4.5, 5, 5.5, 6, or 6.5.

The thermochemical pretreatment may be performed at a pH of 7.5 to 12. The thermochemical pretreatment may be performed at a pH of at least 7.5. The thermochemical pretreatment may be performed at a pH of at most 12. The thermochemical pretreatment may be performed at a pH of 7.5 to 8, 7.5 to 8.5, 7.5 to 9, 7.5 to 9.5, 7.5 to 10, 7.5 to 10.5, 7.5 to 11, 7.5 to 11.5, 7.5 to 12, 8 to 8.5, 8 to 9, 8 to 9.5, 8 to 10, 8 to 10.5, 8 to 11, 8 to 11.5, 8 to 12, 8.5 to 9, 8.5 to 9.5, 8.5 to 10, 8.5 to 10.5, 8.5 to 11, 8.5 to 11.5, 8.5 to 12, 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. The thermochemical pretreatment may be performed at a pH of about 7.5, 8, 8.5, 9, 9.5, 10, 10.5, 11, 11.5, or 12.

The thermochemical pretreatment may be performed at a temperature of 50° C. to 150° C. The thermochemical pretreatment may be performed at a temperature of at least 50° C. The thermochemical pretreatment may be performed at a temperature of at most 150° C. The thermochemical pretreatment may be performed at a temperature of 50° C. to 75° C., 50° C. to 80° C., 50° C. to 90° C., 50° C. to 100° C., 50° C. to 120° C., 50° C. to 130° C., 50° C. to 150° C., 75° C. to 80° C., 75° C. to 90° C., 75° C. to 100° C., 75° C. to 120° C., 75° C. to 130° C., 75° C. to 150° C., 80° C. to 90° C., 80° C. to 100° C., 80° C. to 120° C., 80° C. to 130° C., 80° C. to 150° C., 90° C. to 100° C., 90° C. to 120° C., 90° C. to 130° C., 90° C. to 150° C., 100° C. to 120° C., 100° C. to 130° C., 100° C. to 150° C., 120° C. to 130° C., 120° C. to 150° C., or 130° C. to 150° C. The thermochemical pretreatment may be performed at a temperature of at least 50° C., 75° C., 80° C., 90° C., 100° C., 120° C., 130° C., or 150° C.

The thermochemical treatment may be performed for 0.5 hours to 4 hours. The thermochemical treatment may be performed for at least 0.5 hours. The thermochemical treatment may be performed for at most 4 hours. The thermochemical treatment may be performed for 0.5 hours to 0.75 hours, 0.5 hours to 1 hour, 0.5 hours to 1.5 hours, 0.5 hours to 2 hours, 0.5 hours to 2.5 hours, 0.5 hours to 3 hours, 0.5 hours to 3.5 hours, 0.5 hours to 4 hours, 0.75 hours to 1 hour, 0.75 hours to 1.5 hours, 0.75 hours to 2 hours, 0.75 hours to 2.5 hours, 0.75 hours to 3 hours, 0.75 hours to 3.5 hours, 0.75 hours to 4 hours, 1 hour to 1.5 hours, 1 hour to 2 hours, 1 hour to 2.5 hours, 1 hour to 3 hours, 1 hour to 3.5 hours, 1 hour to 4 hours, 1.5 hours to 2 hours, 1.5 hours to 2.5 hours, 1.5 hours to 3 hours, 1.5 hours to 3.5 hours, 1.5 hours to 4 hours, 2 hours to 2.5 hours, 2 hours to 3 hours, 2 hours to 3.5 hours, 2 hours to 4 hours, 2.5 hours to 3 hours, 2.5 hours to 3.5 hours, 2.5 hours to 4 hours, 3 hours to 3.5 hours, 3 hours to 4 hours, or 3.5 hours to 4 hours. The thermochemical treatment may be performed for at least 0.5 hours, 0.75 hours, 1 hour, 1.5 hours, 2 hours, 2.5 hours, 3 hours, 3.5 hours, or 4 hours.

The thermochemically treated biomass may comprise less than 1% w/w to 30% w/w monosaccharides. The thermochemically treated biomass may comprise less than 1% w/w to 2% w/w, 1% w/w to 5% w/w, 1% w/w to 10% w/w, 1% w/w to 15% w/w, 1% w/w to 20% w/w, 1% w/w to 25% w/w, 1% w/w to 30% w/w, 2% w/w to 5% w/w, 2% w/w to 10% w/w, 2% w/w to 15% w/w, 2% w/w to 20% w/w, 2% w/w to 25% w/w, 2% w/w to 30% w/w, 5% w/w to 10% w/w, 5% w/w to 15% w/w, 5% w/w to 20% w/w, 5% w/w to 25% w/w, 5% w/w to 30% w/w, 10% w/w to 15% w/w, 10% w/w to 20% w/w, 10% w/w to 25% w/w, 10% w/w to 30% w/w, 15% w/w to 20% w/w, 15% w/w to 25% w/w, 15% w/w to 30% w/w, 20% w/w to 25% w/w, 20% w/w to 30% w/w, or 25% w/w to 30% w/w monosaccharides. The thermochemically treated biomass may comprise less than 1% w/w, 2% w/w, 5% w/w, 10% w/w, 15% w/w, 20% w/w, 25% w/w, or 30% w/w monosaccharides.

The thermochemically treated biomass may comprise 5% w/w to 50% w/w disaccharides. The thermochemically treated biomass may comprise 5% w/w to 10% w/w, 5% w/w to 15% w/w, 5% w/w to 20% w/w, 5% w/w to 25% w/w, 5% w/w to 30% w/w, 5% w/w to 35% w/w, 5% w/w to 40% w/w, 5% w/w to 50% w/w, 10% w/w to 15% w/w, 10% w/w to 20% w/w, 10% w/w to 25% w/w, 10% w/w to 30% w/w, 10% w/w to 35% w/w, 10% w/w to 40% w/w, 10% w/w to 50% w/w, 15% w/w to 20% w/w, 15% w/w to 25% w/w, 15% w/w to 30% w/w, 15% w/w to 35% w/w, 15% w/w to 40% w/w, 15% w/w to 50% w/w, 20% w/w to 25% w/w, 20% w/w to 30% w/w, 20% w/w to 35% w/w, 20% w/w to 40% w/w, 20% w/w to 50% w/w, 25% w/w to 30% w/w, 25% w/w to 35% w/w, 25% w/w to 40% w/w, 25% w/w to 50% w/w, 30% w/w to 35% w/w, 30% w/w to 40% w/w, 30% w/w to 50% w/w, 35% w/w to 40% w/w, 35% w/w to 50% w/w, or 40% w/w to 50% w/w disaccharides. The thermochemically treated biomass may comprise less than 5% w/w, 10% w/w, 15% w/w, 20% w/w, 25% w/w, 30% w/w, 35% w/w, 40% w/w, or 50% w/w disaccharides.

### II. Enzyme Treatment and Downstream Processing

The methods of the present disclosure may also include contacting, in a solution or suspension, one or more polysaccharide-cleaving enzymes and the thermochemically treated biomass to form one or more oligosaccharides. Furthermore, the methods may include enriching the solution or suspension to increase the concentration of the one or more oligosaccharides to form the ingredient. The one or more oligosaccharides may be purified from the solution or suspension as provided herein.

One or more steps of the method of forming or manufacturing the composition may be an enzymatic reaction, in which one or more enzymes are placed in a suitable reaction vessel together with one or more feedstocks or biomasses (e.g., plant biomasses), which may be soluble or insoluble in water, and a suitable solvent. As used herein, the term “plant biomass” may be replaced with the terms “feedstock” or “biomass” (e.g., a biomass not derived from a plant) unless indicated otherwise.

A variety of enzymes may be suitable for use in the enzymatic reaction. Any enzyme which produces oligosaccharides when acting on a polysaccharide-containing feedstock may be suitable. For example, the enzymatic reaction may comprise a cellulase, an endo-glucanase, a cellobiohydrolase, a lytic polysaccharide monooxygenase (LPMO), a lichenase, a xyloglucan endoglucanase (XEG), a mannanase, a chitinase, a xylanase, and/or one or more suitable enzymes.

In various cases, the enzymatic reaction may comprise a cellulolytic preparation from a species, such as Trichoderma reesei, which may be purified and/or pretreated and/or may be supplemented with one or more additional enzymes, for example, adding a beta-glucanase, a beta-xylanase, and a cellobiohydrolase; a beta-glucanase, a beta-xylanase, an LPMO, and a cellobiohydrolase; an LPMO and a xylanase; or an LPMO, a xylanase, and a lichenase. Each enzyme may be provided to the enzymatic reaction as a purified enzyme, a semi-purified mixture derived from some natural source or lab-grown culture, in the form of a microbial strain engineered to produce the enzyme, or in any other suitable manner. Fusions of these enzymes, either with other enzymes or with non-enzymatic modules such as carbohydrate-binding modules (CBMs), are also envisaged. For example, an LPMO fused to a CBM, a xylanase fused to a CBM, or a xylanase fused to an LPMO may be utilized.

Aerobic conditions may be used for the one or more enzymatic reactions. Aerobic condition may comprise the addition of oxygen, which may be provided by aeration of the substrate mixture with an oxygen-comprising gas, such as air. Aeration may be conducted by the introduction of oxygen-comprising air bubbles into the aqueous substrate mixtures by various systems, such as an air-injector, an aeration frit, a membrane system, or an internal-loop airlift reactor. In some cases, the concentration of molecular oxygen in the enzymatic reaction may be from about 4 mg/L to about 14 mg/L.

Another exemplary enzyme is a lichenase, which may be selected from the GH5, GH7, GH8, GH9, GH12, GH16, GH17, or GH26 families. For example, GH16 enzyme such as a GH16 enzyme derived from Bacillus subtilis may be utilized. The enzyme may be able to act on, for example, mixed-linkage glucans, which are glucans comprising a mixture of β-1,3 and β-1,4 linkages, and may cleave them at β-1,4 glycosidic bonds. In the case in which the lichenase acts on a mixed-linkage glucan, the β-glucans produced may fall largely within the size range of from 3 to about 7 residues, so they may be particularly useful in the food, cosmetics, and nutraceutical industries. Mixed-linkage glucans are abundant in members of the grass and horsetail families, and as such, grass-based feedstocks such as straw generally have high levels of mixed-linkage glucans and may be acted upon usefully with lichenases. The lichenases may include GH5 lichenase from Bacillus subtilis.

Another alternative enzyme is a xylanase, which may act on, for example, feedstocks comprising a xylan backbone. The xylanase may be, for example, a glucuronoxylanase, an arabinoxylanase, or a glucuronoarabinoxylanase. The enzyme may be active on a variety of polymers having a xylan backbone, such as glucuronoxylan, arabinoxylan, and glucuronoarabinoxylan. These polymers are generally abundant in various plant-derived feedstocks, for example, both hardwood and softwood may comprise suitable polysaccharides, with hardwood often comprising glucuronoxylan and softwood often comprising arabinoglucuronoxylan. Xylanases may include GH5 xylanases from Ruminiclostridium thermocellum and Gonapodya prolifera, and GH30 xylanases from Dickeya chrysanthemi, Bacillus subtilis, Bacteroides ovatus, and Trichoderma reesei.

Another alternative enzyme is a mannanase, which may act on, for example, feedstocks comprising a mannan backbone. The mannanase may be, for example, a mannanase, an glucomannanase, a galactomannanase or a galactoglucomannanase. The enzyme may be active on a variety of polymers having a mannan backbone, such as mannan, glucomannan, galactomannan, or galactoglucomannan. These polymers are generally abundant in various plant-derived feedstocks, for example, both hardwood and softwood may comprise appropriate polysaccharides. Suitable mannanases can include GH5 mannanases from Trichoderma reesei and Aspergillus niger and a GH26 mannanase from Aspergillus niger.

Other enzymes may include xyloglucanases and xyloglucan endoglucanases (XEGs), which are produced by numerous organisms, including plant-pathogenic microbes. Xyloglucanases and XEGs may be able to act on xyloglucan, a hemicellulosic β-1,4 glucan chain abundant in the primary cell wall of higher plants, which is decorated with xylose, some of the xylose residues being further decorated with other residues, such as galactose. When appropriate xyloglucanases or XEGs act on xyloglucan, the products may comprise xyloglucan oligosaccharides having a main chain of a length useful in the foodstuff, cosmetics, and nutraceutical industries. Suitable xyloglucanases may include a GH5 xyloglucanase from Bacteroides ovatus and a GH74 xyloglucanase from Trichoderma reesei.

The enzymatic reaction may take place in solution and/or suspension. The enzymatic reaction may take place in a suitable reaction vessel. In some cases, the enzymatic reaction may take place at a temperature or temperature protocol suitable for the particular combination of enzyme and feedstock, the reaction may be allowed to progress for a certain amount of time (e.g., a predetermined amount of time) until the products have reached a desired concentration or until some other requirement has been met.

In order to ensure optimal contact between the enzymes and feedstock, the reaction mixture may be agitated, either constantly or at intervals. The agitation may take the form of (i) rhythmically moving the entire reaction vessel, (ii) a fan or other stirring device, (iii) a bubble sparging, or (iv) any other suitable method of agitation.

The enzymatic reaction may be a microbial fermentation. The temperature and reaction time may be suitable for the growth of the microbial organism used. The microbial organism may be genetically altered to produce an enzyme suitable for the production of an oligosaccharide composition. The microbe may be a bacterium, for example, Escherichia coli or a fungus, such as Saccharomyces cerevisiae or Trichoderma reesei.

In some embodiments, an expression vector suitable for modifying the subject microorganism may be used such that it produces an enzyme or mixture of enzymes as described elsewhere herein. Where desired, the expression vector may be a plasmid or any other nucleic acid able to induce production of the enzyme. In some instances, the expression vector may comprise one or more of the following regulatory sequences so as to control the expression of the exogenous enzyme: regulatory sequences of a heat shock gene, regulatory sequences of a toxicity gene, regulatory sequences of a spore formation gene, or any other suitable regulatory sequence.

The enzymatic reaction can be carried out at a temperature or temperature protocol suitable for the enzymes and substrates used. For example, the enzymatic reaction may be carried out at a constant temperature in the range of from 10° C. to 100° C., from 20° C. to 80° C., or from 40° C. to 60° C. In some cases, if the enzymatic reaction takes the form of a microbial fermentation, wherein the temperature may be appropriate for such. For example, the enzymatic reaction may comprise the growth of E. coli and/or the temperature may be substantially constant and at about 37° C.

The pH of the solution or suspension may affect the activity of the enzymes. Control of pH may aid in assuring that an enzymatic reaction proceeds at a suitable rate. The enzymatic reaction may take place at a pH in the range of from 2 to 10, 3 to 8, or 4 to 6.

The enzymatic reaction may be allowed to continue for a certain time period before being quenched and the products isolated or otherwise collected. This time period may be from 1 minute to 6 days, 0.5 days to 5 days, or 16 hours to 96 hours. The reaction may alternatively be allowed to proceed until no further catalysis occurs.

The one or more feedstocks added to the enzymatic reaction may comprise polysaccharides. Such polysaccharides may have been produced by a separate reaction proceeding simultaneously, or substantially simultaneously, in the reaction vessel. The polysaccharides present in the enzymatic reaction may be partially cleaved by enzymes into useful oligosaccharides, leaving partially cleaved or uncleaved polysaccharides, which may include, but are not limited to, cellulose, xylan (such as glucuronoxylan, arabinoxylan, or glucuronoarabinoxylan), mannan (such as glucomannan, galactomannan, or galactoglucomannan), mixed-linkage glucan, xyloglucan chitin, chitosan, or lignocellulose.

The enzymatic reaction may be allowed to continue to run until there is from 5% to 75%, 5% to 70%, 5% to 65%, 5% to 55%, or 10% to 50% undigested polysaccharide-containing feedstocks remaining. This can be monitored or checked by reducing end assays, such as the anthrone assay and/or by chromatographic methods such as thin-layer chromatography and/or high-performance anion exchange chromatography.

Any substance which comprises appropriate polysaccharides may form part of the feedstock. As the foodstuff, cosmetic, and nutraceutical industries generally use a broad variety of oligosaccharides, the polysaccharides appropriate for taking part in the enzymatic reaction are not particularly limited. Feedstocks suitable for producing the oligosaccharide profile may comprise, for example, cellulose, lignocellulose, chitin, chitosan, xylan (such as glucuronoxylan, arabinoxylan, and glucuronoarabinoxylan) and/or mannan (such as glucomannan, galactomannan, or galactoglucomannan), however, any feedstock which can be suitably acted upon is envisaged. The feedstocks may comprise sugar cane, corn stover, corncob, wheat bran, wheat straw, hardwood, softwood, or any other suitable biomass or plant biomass.

The feedstocks comprising such polysaccharides are also not particularly limited, as most plant matter is rich in such polymers. As such, the feedstock may comprise plant biomass such as grain, grain chaff, bean pods, seed coats, and/or other seed materials; seaweeds; corn stover, straw, bagasse, miscanthus, sorghum residue, switch grass, bamboo, and/or other monocotyledonous tissue; water hyacinth, leaf tissue, roots, and/or other vegetative matter; hardwood, hardwood chips, hardwood pulp, softwood, softwood chips, softwood pulp, paper, paper pulp, cardboard, and/or other wood-based feedstocks; crab shells, squid biomass, shrimp shells, and/or other marine biomass, and/or any combination of appropriate feedstocks. The feedstock may comprise wheat straw or wood. As any given natural feedstock is likely to comprise a mixture of different polysaccharides, it will sometimes be the case that a mixture of different enzymes is beneficial. Such a mixture may comprise one or more of any suitable enzyme as discussed herein. For example, such a mixture might comprise an LPMO with an endo-glucanase, a xylanase with a lichenase, a cellobiohydrolase with a mannanase, or an endo-glucanase with a cellobiohydrolase. In some embodiments, the enzyme partners may be present in molar ratios, for example, from 1:100 to 100:1. In addition, as many appropriate feedstocks are recalcitrant, pretreatment of the feedstock is envisaged.

After the enzymatic reaction has progressed to a desired point, the one or more oligosaccharides and the one or more polysaccharides from the enzymatic reaction mixture may be separated. This process can be performed in a variety of ways depending on the composition of the biomass used and the specificity of the enzymes used. As the reaction mixture will often comprise a mixture of soluble oligosaccharides and insoluble polysaccharides, the reaction mixture may be filtered to remove insoluble matter and prepare the soluble oligosaccharide obtained for further processing.

The oligosaccharides may also be separated from the polysaccharides in a number of ways. They may be isolated based on solubility, so that a composition of soluble saccharides only is extracted for further processing, and/or isolated chromatographically to produce a composition with a narrower band of oligosaccharide chain lengths. Isolation may, for example, be based on precipitation, size-exclusion chromatography, ion-exchange chromatography, filtration, ultrafiltration, microfiltration, or nanofiltration. In the case that isolation based on solubility is carried out, the profile of saccharides present in the isolated composition will generally depend on the original enzymatic reaction, as different polysaccharides generally decrease in solubility with length at different rates.

Also envisaged is the further treatment of all or part of the produced oligosaccharides to produce further products before incorporation into a foodstuff, cosmetic, or nutraceutical. This further treatment may comprise any chemical, physical, or enzymatic step, such as reduction, for example, reductive amination where appropriate; oxidation, caramelization, modification with a Schiff base, or via the Maillard reaction, or by any combination of such steps, and may provide different products having properties which are achieved or improved for the desired purpose. For example, the caramelization properties, calorific value, flavor, and color may be modified. The oligosaccharides may also be purified, for example, through precipitation, size-exclusion chromatography, ion-exchange chromatography, filtration, ultrafiltration, microfiltration, or nanofiltration.

Also envisaged is the further treatment of all or part of the produced polysaccharide fraction to produce products with improved properties before incorporation into a foodstuff, cosmetic, or nutraceutical. This further treatment may comprise any chemical, physical, or enzymatic step, such as alkylation or acid-treatment. The polysaccharides may also be purified, for example, through precipitation, size-exclusion chromatography, ion-exchange chromatography, filtration, ultrafiltration, microfiltration, or nanofiltration.

In certain instances, following modification and/or purification of the oligosaccharide and polysaccharide fractions, all or part of the fractions can then be recombined at a ratio of from 1:100 to 1:1 polysaccharide:oligosaccharide, for example, from 1:10 to 1:1, from 1:90 to 1:2, from 1:80 to 1:3, from 1:70 to 1:4, or from 1:60 to 1:5. The specific ratio may depend on the desired properties of the final ingredient as well as the modifications and purifications that have been applied to the fractions. It may not be required to recombine all of the oligosaccharide and polysaccharide isolated from the enzymatic reaction.

The fractions can be recombined in a variety of ways, for example, by mixing a solution comprising all or part of the oligosaccharide fraction and a solution and/or suspension comprising all or part of the polysaccharide fraction, which may further be dried, lyophilized, or condensed in some other way. The fractions may also be recombined by mixing a dry form comprising all or part of the oligosaccharide fraction produced by drying, lyophilization, or condensation in some other way, with a dry form comprising all or part of the polysaccharide fraction, produced by drying, lyophilization, or condensation in some other way.

The oligosaccharide components of the final composition may comprise one or more of any type of oligosaccharide. For example, the oligosaccharide components may comprise cello-oligosaccharides, xylo-oligosaccharides, mixed-linkage glucan oligosaccharides, manno-oligosaccharides, xyloglucan oligosaccharides, chito-oligosaccharides, arabinoxylo-oligosaccharides, or derivatives of any of the aforementioned oligosaccharides.

Any such dry or liquid composition may be deemed an ingredient suitable for incorporation into a foodstuff, cosmetic, or nutraceutical at any stage of this process. This includes compositions that may be deemed to be an intermediate during the method, such as a composition formed after the recombining of the oligosaccharide and polysaccharide fractions prior to any further purification, optimization, drying, dissolving, or any other such steps, as well as including the final composition obtained from the method.

As described herein, dry compositions may be formed by drying and/or lyophilization. The dry compositions can be dissolved into a solution of various liquids including water, syrups, pastes, solvents, alcohols, etc. to form the liquid composition ingredient suitable for incorporation into a foodstuff, cosmetic, or nutraceutical. Liquid compositions may be particularly useful in foods that require a smooth texture such as candy, chocolate, and yogurts.

In some embodiments, following modification and/or purification of the oligosaccharide and polysaccharide fractions, all or part of the fractions may then be recombined at a ratio of from 1:100 to 1:1 polysaccharide:oligosaccharide, for example, from 1:10 to 1:1, from 1:90 to 1:2, from 1:80 to 1:3, from 1:70 to 1:4, or from 1:60 to 1:5. The specific ratio may depend on the desired properties of the final ingredient as well as the modifications and purifications that have been applied to the fractions.

Once a composition of the oligosaccharide products suitable for the application being considered is obtained, and further treatment and/or isolation can be carried out. The derivation of a foodstuff, cosmetic, or nutraceutical from the composition can furnish a broad array of potential uses. The ingredients as described herein, can be useful in applications in which oligosaccharides, sugar, bulking sweeteners, low-intensity sweeteners, or other related food ingredients are conventionally used.

The polysaccharide-cleaving enzymes may be one of cellulase, xylanase, xyloglucanase, endo-glucanase, cellobiohydrolase, mannanase, lichenase, or a lytic polysaccharide monooxygenase (LPMO), for example, selected from the group consisting of AA9, AA10, AA11, AA13, AA14, and AA15. The polysaccharide-cleaving enzyme may be prepared from T. reesei fungi and/or the enzymatic reaction runs until there is 5-75%, 5-65%, or 5-50% undigested polysaccharide-containing feedstocks remaining.

The polysaccharide-cleaving enzymes may be operably linked to a catalytic or non-catalytic module, for example, wherein the polysaccharide-cleaving enzyme may be operably linked to a non-catalytic module and the non-catalytic module is a carbohydrate-binding module.

In various embodiments, after the separating of the one or more oligosaccharides and one or more polysaccharides, the one or more oligosaccharides and one or more polysaccharides may be: purified; and/or undergo chemical, physical, or enzymatic treatment, such as reduction, oxidation, caramelization, or Maillard reaction; and/or may be recombined by combining a dried powder of oligosaccharides with a dried polysaccharide powder.

In some embodiments, the ingredient may comprise three or more oligosaccharides of different molecular weights, wherein the method may comprise forming the three or more oligosaccharides by an enzymatic reaction, wherein the enzymatic reaction comprises the step of contacting, in a solution or suspension, one or more polysaccharide-cleaving enzymes and one or more feedstocks.

### III. Foodstuff, Cosmetic, or Nutraceutical Ingredients

### Compositions

The polysaccharide components of the composition may comprise one or more of any type of polysaccharide. For example, the polysaccharide may comprise cellulose, lignocellulose, xylan, mixed-linkage glucan, mannan, xyloglucan, chitin, chitosan, or derivatives of any of the aforementioned polysaccharides.

The composition or ingredient may comprise various oligosaccharides. The composition may include the oligosaccharides at varying amounts, for example, depending on the desired properties of the composition. In some instances, the composition may comprise at least 20% by dry weight, for example, at least 30% by dry weight, cello-oligosaccharides having a degree of polymerization of from two to six; and/or the composition may comprise at least 20% by dry weight, for example, at least 30% by dry weight, xylo-oligosaccharides having a degree of polymerization of from two to twelve; and/or the composition may comprise at least 20% by dry weight, for example, at least 30% by dry weight, mixed-linkage glucan oligosaccharides having a degree of polymerization of from two to five; and/or the composition may comprise at least 20% by dry weight, for example, at least 30% by dry weight, manno-oligosaccharides having a degree of polymerization of from two to twelve; and/or the composition may comprise at least 20% by dry weight, for example, at least 30% by dry weight, xyloglucan oligosaccharides having a degree of polymerization of from four to twelve, and/or the composition may comprise at least 20% by dry weight, for example, at least 30% by dry weight, chito-oligosaccharides having a degree of polymerization of from two to twelve; and/or the composition may comprise at least 20% by dry weight, for example, at least 30% by dry weight, arabinoxylo-oligosaccharides having a degree of polymerization of from three to fifteen. In certain embodiments, it may be understand that the composition can comprise a maximum of 100% by dry weight of the above oligosaccharides, therefore, the above embodiment, wherein the oligosaccharides are present in at least 20% by dry weight, does not comprise all seven types of oligosaccharides.

In various embodiments, the composition or ingredient may comprise about 5% to about 50% w/w cello-oligosaccharides with a degree of polymerization of from two to six. In certain embodiments, the composition or ingredient may comprise about 5% to about 50%, about 10% to about 40%, about 15% to about 35% w/w cello-oligosaccharides with a degree of polymerization of from two to six. The composition or ingredient may comprise at least 5%, 8%, 10%, 15%, 20%, or 25% w/w cello-oligosaccharides with a degree of polymerization of from two to six. In some embodiments, the composition or ingredient may comprise about 20% to about 90% w/w cello-oligosaccharides with a degree of polymerization of from two to six. In certain embodiments, the composition or ingredient may comprise about 5% to about 95%, about 10% to about 92.5%, about 30% to about 80%, about 40% to about 70%, or about 50% to about 60% w/w cello-oligosaccharides with a degree of polymerization of from two to six.

In various embodiments, the composition or ingredient may comprise about 20% to about 90% w/w xylo-oligosaccharides with a degree of polymerization of from two to five. In certain embodiments, the composition or ingredient may comprise about 5% to about 95%, about 10% to about 92.5%, about 30% to about 80%, about 40% to about 70%, or about 50% to about 60% w/w xylo-oligosaccharides with a degree of polymerization of from two to five. For example, the composition may comprise at least 30% w/w of xylo-oligosaccharides with a degree of polymerization from two to five. The composition or ingredient may comprise at least 5%, 8%, 10%, 15%, 20%, or 25% w/w xylo-oligosaccharides with a degree of polymerization of from two to five.

In certain embodiments, the composition or ingredient may comprise about 0.1% to about 15% w/w arabinoxylo-oligosaccharides with a degree of polymerization of from three to twelve. The composition or ingredient may comprise at least 0.1%, 0.3%, 0.5%, 0.8%, 1%, 2%, 3%, 4%, 5%, 6%, 7%, 8%, 9%, 10%, or 15% w/w arabinoxylo-oligosaccharides with a degree of polymerization of from three to twelve. In various embodiments, the composition or ingredient may comprise about 0.5% to about 25% w/w arabinoxylo-oligosaccharides with a degree of polymerization of from three to fifteen. The composition or ingredient may comprise at least 0.1%, 0.3%, 0.5%, 0.8%, 1%, 2%, 3%, 4%, 5%, 10%, 15%, 20%, 25%, 30%, or 35% w/w arabinoxylo-oligosaccharides with a degree of polymerization of from three to fifteen.

In some embodiments, provided herein is the use of an oligosaccharide mixture in the formation of a foodstuff, cosmetic, or nutraceutical, wherein the oligosaccharide mixture comprises two oligosaccharides selected from the list consisting of:
i) cello-oligosaccharides having a degree of polymerization of from two to six;
ii) xylo-oligosaccharides having a degree of polymerization of from two to twelve;
iii) mixed-linkage glucan oligosaccharides having a degree of polymerization of from two to five;
iv) manno-oligosaccharides having a degree of polymerization of from two to twelve;
v) xyloglucan oligosaccharides having a degree of polymerization of from four to ten;
vi) chito-oligosaccharides having a degree of polymerization of from two to twelve; and/or
vii) arabinoxylo-oligosaccharide having a degree of polymerization of from three to fifteen, wherein the two oligosaccharides may be present in a ratio of from 1:9 to 9:1, 1:4 to 4:1, or 2:3 to 3:2 in relation to each other.

In certain cases, the arabinoxylo-oligosaccharides may comprise at least 0.1% arabinosyl residues. The arabinoxylo-oligosaccharides may comprise at least 0.1%, 0.2%, 0.5%, 1%, 5%, or 10% arabinosyl residues.

The amounts of each of the oligosaccharides may be varied depending on the desired properties of the resulting foodstuff, cosmetic, or nutraceutical. For example, the two oligosaccharides may be present in a ratio of 1:9 to 1:1, 1:2 to 1:1, or 2:3 to 1:1 in relation to each other.

The oligosaccharide mixture may further comprise a third oligosaccharide. The oligosaccharide mixture may comprise a third oligosaccharide and a fourth oligosaccharide. The oligosaccharide mixture may comprise a third oligosaccharide, a fourth oligosaccharide, and a fifth oligosaccharide. The oligosaccharide mixture may further comprise a third oligosaccharide, a fourth oligosaccharide, a fifth oligosaccharide, and a sixth oligosaccharide. The oligosaccharide mixture may further comprise a third oligosaccharide, a fourth oligosaccharide, a fifth oligosaccharide, a sixth oligosaccharide, and a seventh oligosaccharide. These oligosaccharides may be selected from the same list as the at least two oligosaccharides as provided above.

Oligosaccharide mixtures of the at least two oligosaccharides may comprise the cello-oligosaccharides, for instance, cello-oligosaccharides in combination with the xylo-oligosaccharides. An alternative composition may comprise cello-oligosaccharides in combination with manno-oligosaccharides. In some embodiments, the oligosaccharide mixtures may include cello-oligosaccharides, xylo-oligosaccharides, and arabinoxylo-oligosaccharides in combination with each other.

The oligosaccharide mixtures of the at least two oligosaccharides may additionally include a polysaccharide, for example, a cellulosic polysaccharide, such as cellulose, or a polysaccharide derivative, for example, a cellulose derivative, such as carboxymethylcellulose, or a polysaccharide aggregate, for example, a portion of lignocellulosic biomass. In some instances, the ratio in the combination may be from 1:100 to 1:1 polysaccharide/polysaccharide derivative/polysaccharide aggregate:oligosaccharide, for example, from 1:90 to 1:2, from 1:80 to 1:3, from 1:70 to 1:4, or from 1:60 to 1:5. As such, the ratio between the first oligosaccharide, the second oligosaccharide, and the polysaccharide may be from 2:2:1 to 30:30:1, for example, about 3:3:1.

### Combinations of Oligosaccharides

A composition may comprise a mixture of one or more oligosaccharides. A mixture of oligosaccharides may comprise two forms or types of oligosaccharides, for instance, cello-oligosaccharides and xylo-oligosaccharides. A mixture of oligosaccharides may comprise three forms of oligosaccharides, for instance, cello-oligosaccharides, manno-oligosaccharides, and xylo-oligosaccharides. A mixture of oligosaccharides may comprise four forms of oligosaccharides, for instance, cello-oligosaccharides, manno-oligosaccharides, mixed-linkage glucan oligosaccharides, and chito-oligosaccharides.

An oligosaccharide mixture may comprise two forms of oligosaccharides, for example, a first oligosaccharide and a second oligosaccharide. An oligosaccharide mixture may comprise about 5% of a first oligosaccharide and about 95% of a second oligosaccharide w/w. An oligosaccharide mixture may comprise about 10% of a first oligosaccharide and about 90% of a second oligosaccharide w/w. An oligosaccharide mixture may comprise about 15% of a first oligosaccharide and about 85% of a second oligosaccharide w/w. An oligosaccharide mixture may comprise about 20% of a first oligosaccharide and about 80% of a second oligosaccharide w/w. An oligosaccharide mixture may comprise about 25% of a first oligosaccharide and about 75% of a second oligosaccharide w/w. An oligosaccharide mixture may comprise about 30% of a first oligosaccharide and about 70% of a second oligosaccharide w/w. An oligosaccharide mixture may comprise about 35% of a first oligosaccharide and about 65% of a second oligosaccharide w/w. An oligosaccharide mixture may comprise about 40% of a first oligosaccharide and about 50% of a second oligosaccharide w/w. An oligosaccharide mixture may comprise 45% of a first oligosaccharide and 55% of a second oligosaccharide w/w. An oligosaccharide mixture may comprise 50% of a first oligosaccharide and 50% of a second oligosaccharide w/w. An oligosaccharide mixture may comprise 55% of a first oligosaccharide and 45% of a second oligosaccharide w/w. An oligosaccharide mixture may comprise 60% of a first oligosaccharide and 30% of a second oligosaccharide w/w. An oligosaccharide mixture may comprise 65% of a first oligosaccharide and 35% of a second oligosaccharide w/w. An oligosaccharide mixture may comprise 70% of a first oligosaccharide and 30% of a second oligosaccharide w/w. An oligosaccharide mixture may comprise 75% of a first oligosaccharide and 25% of a second oligosaccharide w/w. An oligosaccharide mixture may comprise 80% of a first oligosaccharide and 20% of a second oligosaccharide w/w. An oligosaccharide mixture may comprise 85% of a first oligosaccharide and 15% of a second oligosaccharide w/w. An oligosaccharide mixture may comprise 90% of a first oligosaccharide and 10% of a second oligosaccharide w/w. An oligosaccharide mixture may comprise 95% of a first oligosaccharide and 5% of a second oligosaccharide w/w. In some cases, a first oligosaccharide may be cello-oligosaccharides and a second oligosaccharide may be xylo-oligosaccharides. In some instances, a first oligosaccharide may be cello-oligosaccharides and a second oligosaccharide may be manno-oligosaccharides. In some embodiments, a first oligosaccharide may be xylo-oligosaccharides and a second oligosaccharide may be manno-oligosaccharides. Other combinations of a first oligosaccharide and a second oligosaccharide are also within the scope of this disclosure.

An oligosaccharide mixture may comprise three forms of oligosaccharides, for example a first oligosaccharide, a second oligosaccharide, and a third oligosaccharide. An oligosaccharide mixture may comprise about 20% of a first oligosaccharide, 40% of a second oligosaccharide, and 40% of a third oligosaccharide w/w. An oligosaccharide mixture may comprise about 30% of a first oligosaccharide, 30% of a second oligosaccharide, and 40% of a third oligosaccharide w/w. An oligosaccharide mixture may comprise about 10% of a first oligosaccharide, 10% of a second oligosaccharide, and 80% of a third oligosaccharide w/w. An oligosaccharide mixture may comprise about 20% of a first oligosaccharide, 20% of a second oligosaccharide, and 60% of a third oligosaccharide w/w. An oligosaccharide mixture may comprise about 20% of a first oligosaccharide, 30% of a second oligosaccharide, and 50% of a third oligosaccharide w/w. In some examples, a first oligosaccharide may be manno-oligosaccharides, a second oligosaccharide may be xylo-oligosaccharides, and a third oligosaccharide may be cello-oligosaccharides. In some examples, a first oligosaccharide may be xyloglucan-oligosaccharides, a second oligosaccharide may be xylo-oligosaccharides, and a third oligosaccharide may be cello-oligosaccharides. Other combinations of a first oligosaccharide, a second oligosaccharide, and a third oligosaccharide are also within the scope of this disclosure.

An oligosaccharide mixture may comprise two or more oligosaccharides, a first oligosaccharide and a second oligosaccharide which is different than the first oligosaccharide. For instance, the first oligosaccharide may be a xylo-oligosaccharide or a cello-oligosaccharide or a manno-oligosaccharide or other oligosaccharide as provided herein, whereas the second oligosaccharide can be a xylo-oligosaccharide or a cello-oligosaccharide or a manno-oligosaccharide or other oligosaccharides not used as the first oligosaccharide. Stated another way, the first oligosaccharide can be different than the second oligosaccharide (e.g., the first oligosaccharide can be of a different type of oligosaccharide than the second oligosaccharide). The ratio of a first oligosaccharide to a second oligosaccharide in the mixture may be about 1:1, 1:2, 1:3, 1:4, 1:5, 1:6, 1:7, 1:8, or 1:9.

The ratio of a first oligosaccharide to a second oligosaccharide in the mixture may be about 2:1, 2:3, 2:5, 2:7, or 2:9. The oligosaccharides may be cello-oligosaccharides, manno-oligosaccharides, xylo-oligosaccharides, xyloglucan-oligosaccharides, mixed-linkage oligosaccharides, chito-oligosaccharides, arabinoxylo-oligosaccharides, or other oligosaccharides as provided herein, wherein the first oligosaccharide is selected to be a different oligosaccharide than the second oligosaccharide. In other words, the first oligosaccharide may be a different type of oligosaccharide than the second oligosaccharide.

The ratio of a first oligosaccharide to a second oligosaccharide in the mixture may be about 3:1, 3:2, 3:4, 3:5, 3:7, or 3:8. The oligosaccharides may be cello-oligosaccharides, manno-oligosaccharides, xylo-oligosaccharides, xyloglucan-oligosaccharides, mixed-linkage oligosaccharides, chito-oligosaccharides, arabinoxylo-oligosaccharides, or other oligosaccharides provided herein, wherein the first oligosaccharide is selected to be a different oligosaccharide than the second oligosaccharide.

The ratio of a first oligosaccharide to a second oligosaccharide in an oligosaccharide mixture comprising two or more oligosaccharides may be from 1:9 to 9:1, from 1:4 to 4:1, from 1:3 to 3:1, or from 2:3 to 3:2. The oligosaccharides may be cello-oligosaccharides, manno-oligosaccharides, xylo-oligosaccharides, xyloglucan-oligosaccharides, mixed-linkage oligosaccharides, chito-oligosaccharides, arabinoxylo-oligosaccharide, or other oligosaccharides provided herein, wherein the first oligosaccharide is selected to be a different oligosaccharide than the second oligosaccharide.

In some cases, the composition or the ingredient may include at least 1% w/w, 2% w/w, 3% w/w, 4% w/w, 5% w/w, 10% w/w, 15% w/w, 20% w/w, 25% w/w, 30% w/w, or more of cellobiose, xylobiose, mannobiose (e.g., Man-β-1,4-Man), Glc-β-1,4-Man, Man-β-1,4-Glc, laminaribiose, gentiobiose, sophorose, maltose, lactose, or sucrose. In certain cases, the composition or the ingredient may include at least 1% w/w, 2% w/w, 3% w/w, 4% w/w, 5% w/w, 10% w/w, 15% w/w, 20% w/w, 25% w/w, 30% w/w, or more of cellotriose, xylotriose, monoarabinosylated xylobiose, monoglucuronosylated xylobiose, maltotriose, mannotriose (e.g., Man-β-1,4-Man-β-1,4-Man), Glc-β-1,4-Man-β-1,4-Man, Man-β-1,4-Glc-β-1,4-Man, Man-β-1,4-Man-β-1,4-Glc, Man-β-1,4-Glc-β-1,4-Glc, Glc-β-1,4-Man-β-1,4-Glc, Glc-β-1,4-Glc-β-1,4-Man, Glc-β-1,3-Glc-β-1,4-Glc, or Glc-β-1,4-Glc-β-1,3-Glc. In certain instances, the composition or the ingredient may include at least 1% w/w, 2% w/w, 3% w/w, 4% w/w, 5% w/w, 10% w/w, 15% w/w, 20% w/w, 25% w/w, 30% w/w, or more of xylotetraose, cellotetraose, monoarabinosylated xylotriose, monoglucuronosylated xylotriose, diarabinosylated xylobiose, diglucuronosylated xylobiose, maltotetraose, mannotetraose (e.g., Man-β-1,4-Man-β-1,4-Man-β-1,4-Man), Glc-β-1,4-Man-β-1,4-Man-β-1,4-Man, Man-β-1,4-Glc-β-1,4-Man-β-1,4-Man, Man-β-1,4-Man-β-1,4-Glc-β-1,4-Man, Man-β-1,4-Man-β-1,4-Man-β-1,4-Glc, Glc-β-1,4-Glc-β-1,4-Man-β-1,4-Man, Man-β-1,4-Glc-β-1,4-Glc-β-1,4-Man, Man-β-1,4-Man-β-1,4-Glc-β-1,4-Glc, Glc-β-1,4-Man-β-1,4-Glc-β-1,4-Man, Glc-β-1,4-Man-β-1,4-Man-β-1,4-Glc, Man-β-1,4-Glc-β-1,4-Man-β-1,4-Glc, Glc-β-1,3-Glc-β-1,4-Glc-1,4-Glc, Glc-β-1,4-Glc-β-1,3-Glc-1,4-Glc, Glc-β-1,4-Glc-β-1,4-Glc-1,3-Glc, or Glc-β-1,3-Glc-β-1,4-Glc-1,3-Glc. In certain cases, the composition or the ingredient may include at least 0.01% w/w, 0.05% w/w, 0.1% w/w, 0.5% w/w, 1% w/w, 2% w/w, 5% w/w, 10% w/w, 15% w/w, 20% w/w, or more of xylopentaose, cellopentaose, monoarabinosylated xylotetraose, monoglucuronosylated xylotetraose, diarabinosylated xylotriose, diglucuronosylated xylotriose, maltopentaose, mannopentaose (e.g., Man-β-1,4-Man-β-1,4-Man-β-1,4-Man-β-1,4-Man), mixed-linkage glucan-derived pentasaccharide, or mannan-derived pentasaccharide

The composition or ingredient may comprise from 1% to 50%, 5% to 40% 10% to 30%, or 15% to 25% w/w of cellobiose. The composition or ingredient may comprise from 2.5% to 90%, 5% to 80% 10% to 70%, or 20% to 60% w/w of xylobiose. The composition or ingredient may comprise from 2.5% to 75%, 5% to 50% 10% to 40%, or 20% to 30% w/w of xylotriose.

Oligosaccharide Compositions with Varying Degrees of Polymerization

The average degree of polymerization of the oligosaccharides in the composition may be from 1 to 50, 1.5 to 25, 2 to 15, 2.1 to 10, 2.1 to 7, or 2.2 to 5.

The concentration of xylo-oligosaccharides with a degree of polymerization of two in a xylo-oligosaccharide mixture may be from about 2% to about 80% w/w. The concentration of xylo-oligosaccharides with a degree of polymerization of two may be at least 2%, 4%, 6%, 8%, 10%, 12%, 15%, 18%, 20%, 25%, or 30% w/w. The concentration of xylo-oligosaccharides with a degree of polymerization of two may be higher in some cases, for instance, up to 35%, 40%, 45%, 50%, 55%, 60%, 65%, 70%, 75%, or 80% w/w.

The concentration of xylo-oligosaccharides with a degree of polymerization of three in a xylo-oligosaccharide mixture may be about 2% to about 20% w/w. The concentration of xylo-oligosaccharides with a degree of polymerization of three may be at least 2%, 4%, 6%, 8%, 10%, 12%, 15%, 18%, or 20% w/w.

The concentration of xylo-oligosaccharides with a degree of polymerization of four in a xylo-oligosaccharide mixture may be about 5% to about 20% w/w. The concentration of xylo-oligosaccharides with a degree of polymerization of four may be at least 5%, 8%, 10%, 12%, 15%, 18%, or 20% w/w.

The concentration of xylo-oligosaccharides with a degree of polymerization of five in a xylo-oligosaccharide mixture may be about 5% to about 20% w/w. The concentration of xylo-oligosaccharides with a degree of polymerization of five may be at least 5%, 7%, 8%, 10%, 12%, 15%, 18%, or 20% w/w.

The concentration of xylo-oligosaccharides with a degree of polymerization of six in a xylo-oligosaccharide mixture may be about 5% to about 25% w/w. The concentration of xylo-oligosaccharides with a degree of polymerization of six may be at least 5%, 8%, 10%, 12%, 15%, 18%, 20%, or 25% w/w.

The concentration of xylo-oligosaccharides with a degree of polymerization of seven in a xylo-oligosaccharide mixture may be about 2% to about 20% w/w. The concentration of xylo-oligosaccharides with a degree of polymerization of seven may be at least 2%, 4%, 6%, 8%, 10%, 12%, 15%, 17%, or 20% w/w.

The concentration of xylo-oligosaccharides with a degree of polymerization of eight in a xylo-oligosaccharide mixture may be about 1% to about 15% w/w. The concentration of xylo-oligosaccharides with a degree of polymerization of eight may be at least 1%, 2%, 3%, 4%, 5%, 6%, 7%, 8%, 9%, 10%, or 15% w/w.

The concentration of xylo-oligosaccharides with a degree of polymerization of nine in a xylo-oligosaccharide mixture may be about 2% to about 15% w/w. The concentration of xylo-oligosaccharides with a degree of polymerization of nine may be at least 1%, 2%, 3%, 4%, 5%, 6%, 7%, 8%, 9%, 10%, or 15% w/w.

The concentration of xylo-oligosaccharides with a degree of polymerization of ten in a xylo-oligosaccharide mixture may be about 2% to about 15% w/w. The concentration of xylo-oligosaccharides with a degree of polymerization of ten may be at least 1%, 2%, 3%, 4%, 5%, 6%, 7%, 8%, 9%, 10%, or 15% w/w.

The concentration of xylo-oligosaccharides with a degree of polymerization of eleven in a xylo-oligosaccharide mixture may be about 2% to about 15% w/w. The concentration of xylo-oligosaccharides with a degree of polymerization of eleven may be at least 1%, 2%, 3%, 4%, 5%, 6%, 7%, 8%, 9%, 10%, or 15% w/w.

The concentration of xylo-oligosaccharides with a degree of polymerization of twelve in a xylo-oligosaccharide mixture may be about 2% to about 15% w/w. The concentration of xylo-oligosaccharides with a degree of polymerization of twelve may be at least 1%, 2%, 3%, 4%, 5%, 6%, 7%, 8%, 9%, 10%, or 15% w/w.

The concentration of cello-oligosaccharides with a degree of polymerization of two in a cello-oligosaccharide mixture may be about 2% to about 80% w/w. The concentration of cello-oligosaccharides with a degree of polymerization of two may be at least 2%, 4%, 6%, 8%, 10%, 12%, 15%, 18%, 20%, 25%, or 30% w/w. The concentration of cello-oligosaccharides with a degree of polymerization of two may be higher in some cases, for instance, at least 35%, 40%, 45%, 50%, 55%, 60%, 65%, 70%, 75%, or 80% w/w.

The concentration of cello-oligosaccharides with a degree of polymerization of three in a cello-oligosaccharide mixture may be about 2% to about 20% w/w. The concentration of cello-oligosaccharides with a degree of polymerization of three may be at least 2%, 4%, 6%, 8%, 10%, 12%, 15%, 18%, or 20% w/w.

The concentration of cello-oligosaccharides with a degree of polymerization of four in a cello-oligosaccharide mixture may be about 5% to about 20% w/w. The concentration of cello-oligosaccharides with a degree of polymerization of four may be at least 5%, 8%, 10%, 12%, 15%, 18%, or 20% w/w.

The concentration of cello-oligosaccharides with a degree of polymerization of five in a cello-oligosaccharide mixture may be about 5% to about 20% w/w. The concentration of cello-oligosaccharides with a degree of polymerization of five may be at least 5%, 7%, 8%, 10%, 12%, 15%, 18%, or 20% w/w.

The concentration of cello-oligosaccharides with a degree of polymerization of six in a cello-oligosaccharide mixture may be about 5% to about 25% w/w. The concentration of cello-oligosaccharides with a degree of polymerization of six may be at least 5%, 8%, 10%, 12%, 15%, 18%, 20%, or 25% w/w.

The concentration of manno-oligosaccharides with a degree of polymerization of two in a manno-oligosaccharide mixture may be about 2% to about 30% w/w. The concentration of manno-oligosaccharides with a degree of polymerization of two may be at least 2%, 4%, 6%, 8%, 10%, 12%, 15%, 18%, 20%, 25%, or 30% w/w.

The concentration of manno-oligosaccharides with a degree of polymerization of three in a manno-oligosaccharide mixture may be about 2% to about 20% w/w. The concentration of manno-oligosaccharides with a degree of polymerization of three may be at least 2%, 4%, 6%, 8%, 10%, 12%, 15%, 18%, or 20% w/w.

The concentration of manno-oligosaccharides with a degree of polymerization of four in a manno-oligosaccharide mixture may be about 5% to about 20% w/w. The concentration of manno-oligosaccharides with a degree of polymerization of four may be at least 5%, 8%, 10%, 12%, 15%, 18%, or 20% w/w.

The concentration of manno-oligosaccharides with a degree of polymerization of five in a manno-oligosaccharide mixture may be about 5% to about 20% w/w. The concentration of manno-oligosaccharides with a degree of polymerization of five may be at least 5%, 7%, 8%, 10%, 12%, 15%, 18%, or 20% w/w.

The concentration of manno-oligosaccharides with a degree of polymerization of six in a manno-oligosaccharide mixture may be about 5% to about 25% w/w. The concentration of manno-oligosaccharides with a degree of polymerization of six may be at least 5%, 8%, 10%, 12%, 15%, 18%, 20%, or 25% w/w.

The concentration of manno-oligosaccharides with a degree of polymerization of seven in a manno-oligosaccharide mixture may be about 2% to about 20% w/w. The concentration of manno-oligosaccharides with a degree of polymerization of seven may be at least 2%, 4%, 6%, 8%, 10%, 12%, 15%, 17%, or 20% w/w.

The concentration of manno-oligosaccharides with a degree of polymerization of eight in a manno-oligosaccharide mixture may be about 1% to about 15% w/w. The concentration of manno-oligosaccharides with a degree of polymerization of eight may be at least 1%, 2%, 3%, 4%, 5%, 6%, 7%, 8%, 9%, 10%, or 15% w/w.

The concentration of manno-oligosaccharides with a degree of polymerization of nine in a manno-oligosaccharide mixture may be about 2% to about 15% w/w. The concentration of manno-oligosaccharides with a degree of polymerization of nine may be at least 1%, 2%, 3%, 4%, 5%, 6%, 7%, 8%, 9%, 10%, or 15% w/w.

The concentration of manno-oligosaccharides with a degree of polymerization of ten in a manno-oligosaccharide mixture may be about 2% to about 15% w/w. The concentration of manno-oligosaccharides with a degree of polymerization of ten may be at least 1%, 2%, 3%, 4%, 5%, 6%, 7%, 8%, 9%, 10%, or 15% w/w.

The concentration of manno-oligosaccharides with a degree of polymerization of eleven in a manno-oligosaccharide mixture may be about 2% to about 15% w/w. The concentration of manno-oligosaccharides with a degree of polymerization of eleven may be at least 1%, 2%, 3%, 4%, 5%, 6%, 7%, 8%, 9%, 10%, or 15% w/w.

The concentration of manno-oligosaccharides with a degree of polymerization of twelve in a manno-oligosaccharide mixture may be about 2% to about 15% w/w. The concentration of manno-oligosaccharides with a degree of polymerization of twelve may be at least 1%, 2%, 3%, 4%, 5%, 6%, 7%, 8%, 9%, 10%, or 15% w/w.

The concentration of xyloglucan-oligosaccharides with a degree of polymerization of four in a xyloglucan-oligosaccharide mixture may be about 5% to about 20% w/w. The concentration of xyloglucan-oligosaccharides with a degree of polymerization of four may be at least 5%, 8%, 10%, 12%, 15%, 18%, or 20% w/w.

The concentration of xyloglucan-oligosaccharides with a degree of polymerization of five in a xyloglucan-oligosaccharide mixture may be about 5% to about 20% w/w. The concentration of xyloglucan-oligosaccharides with a degree of polymerization of five may be at least 5%, 7%, 8%, 10%, 12%, 15%, 18%, or 20% w/w.

The concentration of xyloglucan-oligosaccharides with a degree of polymerization of six in a xyloglucan-oligosaccharide mixture may be about 5% to about 25% w/w. The concentration of xyloglucan-oligosaccharides with a degree of polymerization of six may be at least 5%, 8%, 10%, 12%, 15%, 18%, 20%, or 25% w/w.

The concentration of xyloglucan-oligosaccharides with a degree of polymerization of seven in a xyloglucan-oligosaccharide mixture may be about 2% to about 20% w/w. The concentration of xyloglucan-oligosaccharides with a degree of polymerization of seven may be at least 2%, 4%, 6%, 8%, 10%, 12%, 15%, 17%, or 20% w/w.

The concentration of xyloglucan-oligosaccharides with a degree of polymerization of eight in a xyloglucan-oligosaccharide mixture may be about 1% to about 15% w/w. The concentration of xyloglucan-oligosaccharides with a degree of polymerization of eight may be at least 1%, 2%, 3%, 4%, 5%, 6%, 7%, 8%, 9%, 10%, or 15% w/w.

The concentration of xyloglucan-oligosaccharides with a degree of polymerization of nine in a xyloglucan-oligosaccharide mixture may be about 2% to about 15% w/w. The concentration of xyloglucan-oligosaccharides with a degree of polymerization of nine may be at least 1%, 2%, 3%, 4%, 5%, 6%, 7%, 8%, 9%, 10%, or 15% w/w.

The concentration of xyloglucan-oligosaccharides with a degree of polymerization of ten in a xyloglucan-oligosaccharide mixture may be about 2% to about 15% w/w. The concentration of xyloglucan-oligosaccharides with a degree of polymerization of ten may be at least 1%, 2%, 3%, 4%, 5%, 6%, 7%, 8%, 9%, 10%, or 15% w/w.

The concentration of mixed-linkage glucan-oligosaccharides with a degree of polymerization of two in a mixed-linkage glucan-oligosaccharide mixture may be about 2% to about 30% w/w. The concentration of mixed-linkage glucan-oligosaccharides with a degree of polymerization of two may be at least 2%, 4%, 6%, 8%, 10%, 12%, 15%, 18%, 20%, 25%, or 30% w/w.

The concentration of mixed-linkage glucan-oligosaccharides with a degree of polymerization of three in a mixed-linkage glucan-oligosaccharide mixture may be about 2% to about 20% w/w. The concentration of mixed-linkage glucan-oligosaccharides with a degree of polymerization of three may be at least 2%, 4%, 6%, 8%, 10%, 12%, 15%, 18%, or 20% w/w.

The concentration of mixed-linkage glucan-oligosaccharides with a degree of polymerization of four in a mixed-linkage glucan-oligosaccharide mixture may be about 5% to about 20% w/w. The concentration of mixed-linkage glucan-oligosaccharides with a degree of polymerization of four may be at least 5%, 8%, 10%, 12%, 15%, 18%, or 20% w/w.

The concentration of mixed-linkage glucan-oligosaccharides with a degree of polymerization of five in a mixed-linkage glucan-oligosaccharide mixture may be about 5% to about 20% w/w. The concentration of mixed-linkage glucan-oligosaccharides with a degree of polymerization of five may be at least 5%, 7%, 8%, 10%, 12%, 15%, 18%, or 20% w/w.

The concentration of chito-oligosaccharides with a degree of polymerization of two in a chito-oligosaccharide mixture may be about 2% to about 30% w/w. The concentration of chito-oligosaccharides with a degree of polymerization of two may be at least 2%, 4%, 6%, 8%, 10%, 12%, 15%, 18%, 20%, 25%, or 30% w/w.

The concentration of chito-oligosaccharides with a degree of polymerization of three in a chito-oligosaccharide mixture may be about 2% to about 20% w/w. The concentration of chito-oligosaccharides with a degree of polymerization of three may be at least 2%, 4%, 6%, 8%, 10%, 12%, 15%, 18%, or 20% w/w.

The concentration of chito-oligosaccharides with a degree of polymerization of four in a chito-oligosaccharide mixture may be about 5% to about 20% w/w. The concentration of chito-oligosaccharides with a degree of polymerization of four may be at least 5%, 8%, 10%, 12%, 15%, 18%, or 20% w/w.

The concentration of chito-oligosaccharides with a degree of polymerization of five in a chito-oligosaccharide mixture may be about 5% to about 20% w/w. The concentration of chito-oligosaccharides with a degree of polymerization of five may be at least 5%, 7%, 8%, 10%, 12%, 15%, 18%, or 20% w/w.

The concentration of chito-oligosaccharides with a degree of polymerization of six in a chito-oligosaccharide mixture may be about 5% to about 25% w/w. The concentration of chito-oligosaccharides with a degree of polymerization of six may be at least 5%, 8%, 10%, 12%, 15%, 18%, 20%, or 25% w/w.

The concentration of chito-oligosaccharides with a degree of polymerization of seven in a chito-oligosaccharide mixture may be about 2% to about 20% w/w. The concentration of chito-oligosaccharides with a degree of polymerization of seven may be at least 2%, 4%, 6%, 8%, 10%, 12%, 15%, 17%, or 20% w/w.

The concentration of chito-oligosaccharides with a degree of polymerization of eight in a chito-oligosaccharide mixture may be about 1% to about 15% w/w. The concentration of chito-oligosaccharides with a degree of polymerization of eight may be at least 1%, 2%, 3%, 4%, 5%, 6%, 7%, 8%, 9%, 10%, or 15% w/w.

The concentration of chito-oligosaccharides with a degree of polymerization of nine in a chito-oligosaccharide mixture may be about 2% to about 15% w/w. The concentration of chito-oligosaccharides with a degree of polymerization of nine may be at least 1%, 2%, 3%, 4%, 5%, 6%, 7%, 8%, 9%, 10%, or 15% w/w.

The concentration of chito-oligosaccharides with a degree of polymerization of ten in a chito-oligosaccharide mixture may be about 2% to about 15% w/w. The concentration of chito-oligosaccharides with a degree of polymerization of ten may be at least 1%, 2%, 3%, 4%, 5%, 6%, 7%, 8%, 9%, 10% or 15% w/w.

The concentration of chito-oligosaccharides with a degree of polymerization of eleven in a chito-oligosaccharide mixture may be about 2% to about 15% w/w. The concentration of chito-oligosaccharides with a degree of polymerization of eleven may be at least 1%, 2%, 3%, 4%, 5%, 6%, 7%, 8%, 9%, 10%, or 15% w/w.

The concentration of chito-oligosaccharides with a degree of polymerization of twelve in a chito-oligosaccharide mixture may be about 2% to about 15% w/w. The concentration of chito-oligosaccharides with a degree of polymerization of twelve may be at least 1%, 2%, 3%, 4%, 5%, 6%, 7%, 8%, 9%, 10%, or 15% w/w.

The concentration of arabinoxylo-oligosaccharides with a degree of polymerization of three in an arabinoxylo-oligosaccharide mixture may be about 2% to about 20% w/w. The concentration of arabinoxylo-oligosaccharides with a degree of polymerization of three may be at least 2%, 4%, 6%, 8%, 10%, 12%, 15%, 18%, or 20% w/w.

The concentration of arabinoxylo-oligosaccharides with a degree of polymerization of four in an arabinoxylo-oligosaccharide mixture may be about 5% to about 20% w/w. The concentration of arabinoxylo-oligosaccharides with a degree of polymerization of four may be at least 5%, 8%, 10%, 12%, 15%, 18%, or 20% w/w.

The concentration of arabinoxylo-oligosaccharides with a degree of polymerization of five in an arabinoxylo-oligosaccharide mixture may be about 5% to about 20% w/w. The concentration of arabinoxylo-oligosaccharides with a degree of polymerization of five may be at least 5%, 7%, 8%, 10%, 12%, 15%, 18%, or 20% w/w.

The concentration of arabinoxylo-oligosaccharides with a degree of polymerization of six in an arabinoxylo-oligosaccharide mixture may be about 5% to about 25% w/w. The concentration of arabinoxylo-oligosaccharides with a degree of polymerization of six may be at least 5%, 8%, 10%, 12%, 15%, 18%, 20%, or 25% w/w.

The concentration of arabinoxylo-oligosaccharides with a degree of polymerization of seven in an arabinoxylo-oligosaccharide mixture may be about 2% to about 20% w/w. The concentration of arabinoxylo-oligosaccharides with a degree of polymerization of seven may be at least 2%, 4%, 6%, 8%, 10%, 12%, 15%, 17%, or 20% w/w.

The concentration of arabinoxylo-oligosaccharides with a degree of polymerization of eight in an arabinoxylo-oligosaccharide mixture may be about 1% to about 15% w/w. The concentration of arabinoxylo-oligosaccharides with a degree of polymerization of eight may be at least 1%, 2%, 3%, 4%, 5%, 6%, 7%, 8%, 9%, 10%, or 15% w/w.

The concentration of arabinoxylo-oligosaccharides with a degree of polymerization of nine in an arabinoxylo-oligosaccharide mixture may be about 2% to about 15% w/w. The concentration of arabinoxylo-oligosaccharides with a degree of polymerization of nine may be at least 1%, 2%, 3%, 4%, 5%, 6%, 7%, 8%, 9%, 10%, or 15% w/w.

The concentration of arabinoxylo-oligosaccharides with a degree of polymerization of ten in an arabinoxylo-oligosaccharide mixture may be about 2% to about 15% w/w. The concentration of arabinoxylo-oligosaccharides with a degree of polymerization of ten may be at least 1%, 2%, 3%, 4%, 5%, 6%, 7%, 8%, 9%, 10%, or 15% w/w.

The concentration of arabinoxylo-oligosaccharides with a degree of polymerization of eleven in an arabinoxylo-oligosaccharide mixture may be about 2% to about 15% w/w. The concentration of arabinoxylo-oligosaccharides with a degree of polymerization of eleven may be at least 1%, 2%, 3%, 4%, 5%, 6%, 7%, 8%, 9%, 10%, or 15% w/w.

The concentration of arabinoxylo-oligosaccharides with a degree of polymerization of twelve in an arabinoxylo-oligosaccharide mixture may be about 2% to about 15% w/w. The concentration of arabinoxylo-oligosaccharides with a degree of polymerization of twelve may be at least 1%, 2%, 3%, 4%, 5%, 6%, 7%, 8%, 9%, 10%, or 15% w/w.

The concentration of arabinoxylo-oligosaccharides with a degree of polymerization of thirteen in an arabinoxylo-oligosaccharide mixture may be about 2% to about 15% w/w. The concentration of arabinoxylo-oligosaccharides with a degree of polymerization of thirteen may be at least 1%, 2%, 3%, 4%, 5%, 6%, 7%, 8%, 9%, 10%, or 15% w/w.

The concentration of arabinoxylo-oligosaccharides with a degree of polymerization of fourteen in an arabinoxylo-oligosaccharide mixture may be about 2% to about 15% w/w. The concentration of arabinoxylo-oligosaccharides with a degree of polymerization of fourteen may be at least 1%, 2%, 3%, 4%, 5%, 6%, 7%, 8%, 9%, 10%, or 15% w/w.

The concentration of arabinoxylo-oligosaccharides with a degree of polymerization of fifteen in an arabinoxylo-oligosaccharide mixture may be about 2% to about 15% w/w. The concentration of arabinoxylo-oligosaccharides with a degree of polymerization of fifteen may be at least 1%, 2%, 3%, 4%, 5%, 6%, 7%, 8%, 9%, 10%, or 15% w/w.

The concentration of arabinoxylo-oligosaccharides with a degree of polymerization of from three to twelve in an arabinoxylo-oligosaccharide mixture may be about 0.1% to about 15% w/w. The concentration of arabinoxylo-oligosaccharides with a degree of polymerization of three to twelve may be at least 0.1%, 0.3%, 0.5%, 0.8%, 1%, 2%, 3%, 4%, 5%, 6%, 7%, 8%, 9%, 10%, or 15% w/w.

Compositions with Combinations of Monosaccharides, Polysaccharides, and/or Oligosaccharides

In some embodiments, the composition or ingredient (e.g., the ingredient for human consumption) may soluble in water. The solubility of the ingredient in water may be at least 80 g of the ingredient per 100 g of water at 50° C.

The ingredient may be combined with a liquid to form a liquid ingredient. In some cases, a viscosity of the liquid ingredient may be comparable or similar to a viscosity of corn syrup. In some other cases, that viscosity of the liquid ingredient may comparable or similar to a viscosity of high-fructose corn syrup. For example, the liquid ingredient may have a viscosity of from 5 cps to 100,000 cps, 8,000 cps to 100,000 cps, 10,000 cps to 50,000 cps, or 15,000 cps to 25,000 cps. Moreover, the liquid ingredient may have fewer calories per gram than corn syrup or high-fructose corn syrup. The liquid ingredient may have a lower glycemic index than corn syrup or high-fructose corn syrup.

In some embodiments, the liquid may comprise water or any other suitable liquid. The liquid ingredient may comprises at least 5%, 10%, 20%, 30%, 40%, or 50% by dry weight of the at least one oligosaccharide. Furthermore, the liquid ingredient may comprise at least 0.2%, 0.5%, 1%, 2%, 3%, 5%, or 10% by dry weight of the at least one polysaccharide. For example, the liquid ingredient may comprises at least 20% by dry weight of the at least one oligosaccharide and at least 2% by dry weight of the at least one polysaccharide. Other combinations of the at least one oligosaccharide and the at least one polysaccharide are also within the scope of the present disclosure.

The liquid ingredient comprises at least 0.2%, 0.5%, 1%, 2%, 3%, 5%, or 10% by dry weight of xylan. The liquid ingredient may comprise at least 0.2%, 0.5%, 1%, 2%, 3%, 5%, or 10% by dry weight of mannan. The liquid ingredient may comprise at least 0.2%, 0.5%, 1%, 2%, 3%, 5%, or 10% by dry weight of a cellulose derivative.

In various cases, the liquid ingredient may have a concentration of polysaccharides of from 0.1% to 50%, 0.1% to 40%, 0.1% to 30%, 0.1% to 20%, 0.1% to 10%, 0.5% to 50%, or 1% to 50% w/v. For example, the liquid ingredient may have a concentration of polysaccharides of from 0.1% to 50% w/v. The liquid ingredient may comprise an amount of polysaccharide and oligosaccharide in a ratio from 1:200 to 1:1, 1:150 to 1:1, 1:125 to 1:1 1:100 to 1:1, 1:90 to 1:1, 1:80 to 1:1, 1:70 to 1:1, 1:60 and 1:1, 1:50 and 1:1, 1:25 and 1:1, or 1:10 and 1:1. For example, the liquid ingredient may comprises an amount of polysaccharide and oligosaccharide in a ratio from 1:100 to 1:1.

The one or more soluble polysaccharides may comprise at least one of a mannan, a xylan, a mixed-linkage glucan, a lignocellulose, a hemicellulose, a cellulose derivative, a chitosan, a xyloglucan, or any other suitable soluble polysaccharide. The cellulose derivative may comprise at least one of a cellulose acetate, a hydroxyethylcellulose, a hydroxymethylcellulose, or any other suitable cellulose derivative.

The biomass may comprise at least one of a sugar cane biomass, a corn biomass, a wheat biomass, a hardwood biomass, a softwood biomass, or any other suitable biomass.

In certain instances, a composition for human consumption may include a soluble polysaccharide and an oligosaccharide comprising at least one of (i) a cello-oligosaccharide having a degree of polymerization (DP) of from two to six; (ii) a xylo-oligosaccharide having a DP of from two to twelve; (iii) a manno-oligosaccharide having a DP of from two to twelve; (iv) an arabinoxylo-oligosaccharide having a DP of from three to fifteen; (v) a mixed-linkage glucan oligosaccharide having a DP of from two to five; or (vi) a chito-oligosaccharide having a DP of from two to twelve. The composition may include less than 5% by dry weight soluble polysaccharides. In some cases, the composition may include less than 1%, 2%, 5%, 7.5%, 10%, or 20% by dry weight soluble polysaccharides. In some embodiments, the composition may be free, or substantially free, of insoluble polysaccharides.

A composition may comprise a combination of polysaccharides and oligosaccharides. In some embodiments, a composition may comprise a combination of oligosaccharides and soluble polysaccharides. The source of the polysaccharides (or the soluble polysaccharides) in such compositions may contain cellulose, such as biomass, for example, the undigested component of partially digested biomass, such as the undigested biomass from the same reaction as that which produced the oligosaccharides. The polysaccharides in the undigested biomass may comprise lignin, polyphenol, cellulose, lignocellulose, or any other suitable polysaccharides as described herein. Addition of polysaccharides (e.g., soluble polysaccharides) to oligosaccharide mixtures can be done to improve the gastrointestinal tolerance of the oligosaccharide mixtures. Oligosaccharide consumption can cause gastrointestinal distress, including diarrhea, discomfort, and bloating. The compositions described herein may have an improved gastrointestinal tolerance such as, less or no discomfort, bloating, diarrhea, or gastrointestinal distress as compared to a saccharide composition available commercially or a saccharide composition comprising primarily monosaccharides and/or disaccharides. For example, a subject who ingests one or more of the compositions provided herein may have an improved gastrointestinal tolerance such as, less or no discomfort, bloating, diarrhea, or gastrointestinal distress as compared to if, or when, the subject ingests a saccharide composition available commercially or a saccharide composition comprising primarily monosaccharides and/or disaccharides.

The concentration of undigested biomass in a composition may be from 1% to 50% w/w. The concentration of undigested biomass in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of undigested biomass in a composition may be about 1%, 5%, 10%, 15%, 20%, 25%, 30%, 35%, 40%, 45%, or 50% w/w. The concentration of undigested biomass in a composition may be at least 1%, 5%, 10%, 15%, 20%, 25%, 30%, 35%, 40%, or 45% w/w. The concentration of undigested biomass in a composition may be at most 5%, 10%, 15%, 20%, 25%, 30%, 35%, 40%, 45%, or 50% w/w.

The concentration of soluble polysaccharides in a composition may be from 1% to 50% w/w. The concentration of soluble polysaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of soluble polysaccharides in a composition may be about 1%, 5%, 10%, 15%, 20%, 25%, 30%, 35%, 40%, 45%, or 50% w/w. The concentration of soluble polysaccharides in a composition may be at least 1%, 5%, 10%, 15%, 20%, 25%, 30%, 35%, 40%, or 45% w/w. The concentration of soluble polysaccharides in a composition may be at most 5%, 10%, 15%, 20%, 25%, 30%, 35%, 40%, 45%, or 50% w/w.

The concentration of xylo-oligosaccharides in a composition may be from 1% to 50% w/w. The concentration of xylo-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of xylo-oligosaccharides in a composition may be about 1%, 5%, 10%, 15%, 20%, 25%, 30%, 35%, 40%, 45%, or 50% w/w. The concentration of xylo-oligosaccharides in a composition may be at least 1%, 5%, 10%, 15%, 20%, 25%, 30%, 35%, 40%, or 45% w/w. The concentration of xylo-oligosaccharides in a composition may be at most 5%, 10%, 15%, 20%, 25%, 30%, 35%, 40%, 45%, or 50% w/w.

The concentration of cello-oligosaccharides in a composition may be from 1% to 50% w/w. The concentration of cello-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of cello-oligosaccharides in a composition may be about 1%, 5%, 10%, 15%, 20%, 25%, 30%, 35%, 40%, 45%, or 50% w/w. The concentration of cello-oligosaccharides in a composition may be at least 1%, 5%, 10%, 15%, 20%, 25%, 30%, 35%, 40%, or 45% w/w. The concentration of cello-oligosaccharides in a composition may be at most 5%, 10%, 15%, 20%, 25%, 30%, 35%, 40%, 45%, or 50% w/w.

In some embodiments, the composition may comprise at least 5% w/w of cello-oligosaccharides and at least 5% w/w of a second oligosaccharides (e.g., at least 5% w/w of xylo-oligosaccharides, manno-oligosaccharides, mixed-linkage glucan oligosaccharides, xyloglucan-oligosaccharides, chito-oligosaccharides, arabinoxylo-oligosaccharides, or any other suitable oligosaccharides).

The concentration of manno-oligosaccharides in a composition may be from 1% to 50% w/w. The concentration of manno-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of manno-oligosaccharides in a composition may be about 1%, 5%, 10%, 15%, 20%, 25%, 30%, 35%, 40%, 45%, or 50% w/w. The concentration of manno-oligosaccharides in a composition may be at least 1%, 5%, 10%, 15%, 20%, 25%, 30%, 35%, 40%, or 45% w/w. The concentration of manno-oligosaccharides in a composition may be at most 5%, 10%, 15%, 20%, 25%, 30%, 35%, 40%, 45%, or 50% w/w.

The concentration of chito-oligosaccharides in a composition may be from 1% to 50% w/w. The concentration of chito-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of chito-oligosaccharides in a composition may be about 1%, 5%, 10%, 15%, 20%, 25%, 30%, 35%, 40%, 45%, or 50% w/w. The concentration of chito-oligosaccharides in a composition may be at least 1%, 5%, 10%, 15%, 20%, 25%, 30%, 35%, 40%, or 45% w/w. The concentration of chito-oligosaccharides in a composition may be at most 5%, 10%, 15%, 20%, 25%, 30%, 35%, 40%, 45%, or 50% w/w.

The concentration of xyloglucan-oligosaccharides in a composition may be from 1% to 50% w/w. The concentration of xyloglucan-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of xyloglucan-oligosaccharides in a composition may be about 1%, 5%, 10%, 15%, 20%, 25%, 30%, 35%, 40%, 45%, or 50% w/w. The concentration of xyloglucan-oligosaccharides in a composition may be at least 1%, 5%, 10%, 15%, 20%, 25%, 30%, 35%, 40%, or 45% w/w. The concentration of xyloglucan-oligosaccharides in a composition may be at most 5%, 10%, 15%, 20%, 25%, 30%, 35%, 40%, 45%, or 50% w/w.

The concentration of mixed-linkage glucan-oligosaccharides in a composition may be from 1% to 50% w/w. The concentration of mixed-linkage glucan-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of mixed-linkage glucan-oligosaccharides in a composition may be about 1%, 5%, 10%, 15%, 20%, 25%, 30%, 35%, 40%, 45%, or 50% w/w. The concentration of mixed-linkage glucan-oligosaccharides in a composition may be at least 1%, 5%, 10%, 15%, 20%, 25%, 30%, 35%, 40%, or 45% w/w. The concentration of mixed-linkage glucan-oligosaccharides in a composition may be at most 5%, 10%, 15%, 20%, 25%, 30%, 35%, 40%, 45%, or 50% w/w.

The concentration of arabinoxylo-oligosaccharides in a composition may be from 1% to 50% w/w. The concentration of arabinoxylo-oligosaccharides in a composition may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50% w/w. The concentration of arabinoxylo-oligosaccharides in a composition may be about 1%, 5%, 10%, 15%, 20%, 25%, 30%, 35%, 40%, 45%, or 50% w/w. The concentration of arabinoxylo-oligosaccharides in a composition may be at least 1%, 5%, 10%, 15%, 20%, 25%, 30%, 35%, 40%, or 45% w/w. The concentration of arabinoxylo-oligosaccharides in a composition may be at most 5%, 10%, 15%, 20%, 25%, 30%, 35%, 40%, 45%, or 50% w/w.

A composition may comprise one or more polysaccharides (e.g., one or more soluble polysaccharides) and one or more oligosaccharides. The composition may comprise a polysaccharide and one type of oligosaccharide. The composition may comprise a polysaccharide or plurality of polysaccharides and two forms of oligosaccharides. The composition may comprise a polysaccharide or plurality of polysaccharides and three forms of oligosaccharides. The composition may comprise a polysaccharide or plurality of polysaccharides and four forms of oligosaccharides. The composition may comprise a polysaccharide or plurality of polysaccharides and five forms of oligosaccharides. The oligosaccharides may be xylo-oligosaccharides, cello-oligosaccharides, manno-oligosaccharides, mixed-linkage glucan oligosaccharides, xyloglucan-oligosaccharides, chito-oligosaccharides, arabinoxylo-oligosaccharides, or any other suitable oligosaccharides described herein.

The composition may comprise from about 1% to 50% polysaccharides w/w, such as in the type of undigested biomass or extracted soluble polysaccharides, and about 5% to about 95% oligosaccharides w/w. The composition of polysaccharides may be at least about 1%, 2%, 2.5%, 3%, 4%, 5%, 10%, 15%, 20%, 25%, 30%, 35%, 40%, 45%, or 50% w/w. Oligosaccharides in such mixtures may be present at greater than 5%, 10%, 15%, 20%, 25%, 30%, 35%, 40%, 45%, 50%, 55%, 60%, 65%, 70%, 75%, 80%, 85%, 90%, or 95% w/w. The oligosaccharides may be a mixture of one or more oligosaccharides. For instance, a composition may comprise 5% undigested biomass and 50% oligosaccharide mixture w/w as described elsewhere herein. In another instance, a composition may comprise 2.5% soluble polysaccharides and 50% oligosaccharide mixture w/w as described elsewhere herein.

The composition may comprise about 5% polysaccharides w/w, such as in the type of undigested biomass, and about 5% to about 95% oligosaccharides w/w. Oligosaccharides in such mixtures may be present at greater than 5%, 10%, 15%, 20%, 25%, 30%, 35%, 40%, 45%, 50%, 55%, 60%, 65%, 70%, 75%, 80%, 85%, 90%, or 95% w/w. The oligosaccharides may be a mixture of one or more oligosaccharides. For instance, a composition may comprise 5% undigested biomass and 50% oligosaccharide mixture w/w as described elsewhere herein. In another instance, a composition may comprise 5% soluble polysaccharide(s) and 50% oligosaccharide mixture w/w as described elsewhere herein.

The composition may comprise about 7% polysaccharides w/w, such as in the type of undigested biomass and about 5% to about 93% oligosaccharides w/w. Oligosaccharides may form at least 5%, 10%, 15%, 20%, 25%, 30%, 35%, 40%, 45%, 50%, 55%, 60%, 65%, 70%, 75%, 80%, 85%, 90%, or 93% w/w of such mixtures. The oligosaccharides may be a mixture of one or more oligosaccharides. For instance, a composition may comprise 7% undigested biomass and 50% oligosaccharide mixture w/w as described elsewhere herein. In another instance, a composition may comprise 7% soluble polysaccharide(s) and 50% oligosaccharide mixture w/w as described elsewhere herein.

The composition may comprise about 10% polysaccharides w/w, such as in the type of undigested biomass and about 5% to about 90% oligosaccharides w/w. Oligosaccharides may form at least 5%, 10%, 15%, 20%, 25%, 30%, 35%, 40%, 45%, 50%, 55%, 60%, 65%, 70%, 75%, 80%, 85%, or 90% w/w of such mixtures. The oligosaccharides may be a mixture of one or more oligosaccharides. For instance, a composition may comprise 10% undigested biomass and 50% oligosaccharide mixture w/w as described elsewhere herein. In another instance, a composition may comprise 10% polysaccharide(s) and 50% oligosaccharide mixture w/w as described elsewhere herein.

The composition may comprise about 12% polysaccharides w/w, such as in the type of undigested biomass and from about 5% to about 95% oligosaccharides w/w. Oligosaccharides may form at least 5%, 10%, 15%, 20%, 25%, 30%, 35%, 40%, 45%, 50%, 55%, 60%, 65%, 70%, 75%, 80%, 85%, or 88% w/w of such mixtures. The oligosaccharides may be a mixture of one or more oligosaccharides. For instance, a composition may comprise 12% undigested biomass and 50% oligosaccharide mixture w/w as described elsewhere herein. In another instance, a composition may comprise 12% soluble polysaccharide(s) and 50% oligosaccharide mixture w/w as described elsewhere herein.

The composition may comprise about 15% polysaccharides w/w, such as in the type of undigested biomass and about 5% to about 85% oligosaccharides w/w. Oligosaccharides may form at least 5%, 10%, 15%, 20%, 25%, 30%, 35%, 40%, 45%, 50%, 55%, 60%, 65%, 70%, 75%, 80%, or 85% w/w of such mixtures. The oligosaccharides may be a mixture of one or more oligosaccharides. For instance, a composition may comprise 15% undigested biomass and 50% oligosaccharide mixture w/w as described elsewhere herein. In another instance, a composition may comprise 15% soluble polysaccharide(s) and 50% oligosaccharide mixture w/w as described elsewhere herein.

The composition may comprise about 20% polysaccharides w/w, such as in the type of undigested biomass and about 5% to about 80% oligosaccharides w/w. Oligosaccharides may form at least 5%, 10%, 15%, 20%, 25%, 30%, 35%, 40%, 45%, 50%, 55%, 60%, 65%, 70%, 75%, or 80% w/w of such mixtures. The oligosaccharides may be a mixture of one or more oligosaccharides. For instance, a composition may comprise 20% undigested biomass and 50% oligosaccharide mixture w/w as described elsewhere herein. In another instance, a composition may comprise 20% soluble polysaccharide(s) and 50% oligosaccharide mixture w/w as described elsewhere herein.

The composition may comprise about 25% polysaccharides w/w, such as in the type of undigested biomass and about 5% to about 75% oligosaccharides w/w. Oligosaccharides may form at least 5%, 10%, 15%, 20%, 25%, 30%, 35%, 40%, 45%, 50%, 55%, 60%, 65%, 70%, or 75% w/w of such mixtures. The oligosaccharides may be a mixture of one or more oligosaccharides. For instance, a composition may comprise 25% undigested biomass and 50% oligosaccharide mixture w/w as described elsewhere herein. In another instance, a composition may comprise 25% soluble polysaccharide(s) and 50% oligosaccharide mixture w/w as described elsewhere herein.

The composition may comprise about 30% polysaccharides w/w, such as in the type of undigested biomass and about 5% to about 70% oligosaccharides w/w. Oligosaccharides may form at least 5%, 10%, 15%, 20%, 25%, 30%, 35%, 40%, 45%, 50%, 55%, 60%, 65%, or 70% w/w of such mixtures. The oligosaccharides may be a mixture of one or more oligosaccharides. For instance, a composition may comprise 30% undigested biomass and 50% oligosaccharide mixture w/w as described elsewhere herein. In another instance, a composition may comprise 30% soluble polysaccharide(s) and 50% oligosaccharide mixture w/w as described elsewhere herein.

The composition may comprise about 40% polysaccharides w/w, such as in the type of undigested biomass and about 5% to about 60% oligosaccharides w/w. Oligosaccharides may form at least 5%, 10%, 15%, 20%, 25%, 30%, 35%, 40%, 45%, 50%, 55%, or 60% w/w of such mixtures. The oligosaccharides may be a mixture of one or more oligosaccharides. For instance, a composition may comprise 40% undigested biomass and 50% oligosaccharide mixture w/w as described elsewhere herein. In another instance, a composition may comprise 40% soluble polysaccharide(s) and 50% oligosaccharide mixture w/w as described elsewhere herein.

The composition may comprise about 50% polysaccharides w/w, such as in the type of undigested biomass and about 5% to about 50% oligosaccharides w/w. Oligosaccharides may form at least 5%, 10%, 15%, 20%, 25%, 30%, 35%, 40%, 45%, or 50% w/w of such mixtures. The oligosaccharides may be a mixture of one or more oligosaccharides. For instance, a composition may comprise 50% undigested biomass and 50% oligosaccharide mixture w/w as described elsewhere herein. In another instance, a composition may comprise 50% soluble polysaccharide(s) and 50% oligosaccharide mixture w/w as described elsewhere herein.

In some embodiments, the composition or ingredient may comprise less than 1%, 5%, 10%, 15%, 20%, 25%, 30%, or 40% w/w monosaccharides. For example, the composition may comprise less than 20% w/w monosaccharides. The composition may include from 10% to 40%, 15% to 30%, 18% to 25%, or about 20% w/w monosaccharides. In some embodiments, the composition or ingredient may comprise less than 1%, 5%, 10%, 15%, 20%, 25%, 30%, or 40% w/w glucose. For example, the composition may comprise less than 10% w/w glucose. The composition may include from 10% to 40%, 15% to 30%, 18% to 25%, or about 20% w/w glucose. In some embodiments, the composition or ingredient may comprise less than 1%, 5%, 10%, 15%, 20%, 25%, 30%, or 40% w/w xylose. For example, the composition may comprise less than 10% w/w xylose. The composition may include from 10% to 40%, 15% to 30%, 18% to 25%, or about 20% w/w xylose.

In certain cases, the ratio of glucose residues to xylose residues (e.g., glucose:xylose) within the composition or ingredient may be from 1:1 and 1:9, 1:1 and 1:7, 1:1 and 1:5, 1:1 and 1:3, or 1:1 and 1:2.

In certain embodiments, the composition may comprise less than 30%, 40%, 50%, 60%, 65%, 70%, 75%, or 80% w/w disaccharides. For example, the composition may comprise less than 70% w/w disaccharides. The composition may include from 10% to 95%, 15% to 90%, 20% to 80%, 30% to 70%, or 40% to 60% w/w disaccharides. The composition may comprise from 5% to 95%, 10% to 92.5%, 15% to 90%, 20% to 70%, 30% to 60%, or 40% to 50% disaccharides. In various embodiments, the composition may comprise at least 0.5%, 1%, 2.5%, 5%, 7.5%, 10%, 15%, or 20% w/w trisaccharides. For example, the composition may comprise at least 5% w/w trisaccharides. In various embodiments, the composition may comprise at least 0.5%, 1%, 2.5%, 5%, 7.5%, 10%, 15%, or 20% w/w trisaccharides. For example, the composition may comprise at least 5% w/w trisaccharides. The composition may comprise from 1% to 75%, 2.5% to 60%, 5% to 50%, 10% to 40%, or 20% to 30% trisaccharides. In some cases, the composition may comprise at least 0.1%, 0.5%, 1%, 2.5%, 5%, 7.5%, 10%, 15%, or 20% w/w tetrasaccharides. For example, the composition may comprise at least 1% w/w tetrasaccharides. In various cases, the composition may comprise at least 0.01%, 0.05%, 0.1%, 0.2%, 0.3%, 0.5%, 0.5%, 1%, 2.5%, 5%, 7.5%, or w/w pentasaccharides. For example, the composition may comprise at least 0.1% w/w pentasaccharides.

### Use of Compositions as Ingredients

In some embodiments, the composition is an ingredient (e.g., in a foodstuff). In certain embodiments, the ingredient comprises at least 50%, 60%, 70%, 80%, 90%, 95%, 99%, or 99.5% by dry weight of saccharide present. The ingredient may consist essentially of saccharides. For example, the ingredient may have less than 0.5%, 0.3%, or 0.1% by dry weight of other substances.

The ingredient may comprise an oligosaccharide mixture as described elsewhere herein. The ingredient may comprise at least two of the oligosaccharides. For instance, it may comprise three of the oligosaccharides. It may comprise four oligosaccharides. It may comprise five oligosaccharides. It may comprise six oligosaccharides. It may comprise seven oligosaccharides.

In some embodiments, the ingredient comprises cello-oligosaccharides, for instance, cello-oligosaccharides in combination with xylo-oligosaccharides. An alternative ingredient may comprise cello-oligosaccharides in combination with manno-oligosaccharides.

Ingredients may be used to prepare finished products. The ingredient may also be treated in some physical or chemical way before or during incorporation into a foodstuff, cosmetic, or nutraceutical. It may be directly incorporated into a product, or it may be incorporated into, for example, a dough, cake mixture, chocolate mixture, or other foodstuff precursor; a cosmetic base composition; or a nutraceutical, and, for example, be cooked or otherwise treated in a way which may cause chemical modification, a change of texture, a change of color, or other modification.

A foodstuff, cosmetic, or nutraceutical may be produced from an ingredient described herein. For example, in the food industry, the saccharide formulations produced by the current method may be used as sweeteners, bulking agents, added dietary fiber, or humectants. The ingredient may be used as a sugar substitute. The ingredient may be incorporated into cakes, breads, or other baked goods, or into chocolate or other confectionery such as toffee, fudge, meringue, jam, jelly, or caramel; or drinks, for example, to provide favorable taste or color characteristics or to increase dietary fiber content. In certain instances, the ingredient may be incorporated into animal feed, for example, either as an isolated ingredient or by utilizing the enzymatic reaction mixture directly as feed.

In the cosmetics industry, saccharides can be useful as ingredients, as they may improve texture and moisture retention, act as UV-absorbing molecules, maintain a gel or cream structure, and/or serve as bulking agents. The compositions described herein can be incorporated into nutraceutical compositions, as the dietary fiber they provide can encourage digestive health, well-regulated gut flora, and other benefits to wellbeing. In this context, they may also function as an ingredient in a probiotic drink or other prebiotic or probiotic formulation.

Compositions or ingredients as described herein may be used to alter one or more properties of the finished product. Such properties include, but are not limited to, sweetness, texture, mouthfeel, binding, glazing, smoothness, moistness, viscosity, color, hygroscopicity, flavor, bulking, water-retention, caramelization, surface texture, crystallization, structural properties, and dissolution.

In some cases, the compositions and/or ingredients described herein may provide a property to a finished product which is comparable to or better than the same property as provided by a saccharide mixture comprising primarily monosaccharides and/or disaccharides. The control composition may be a saccharide used commonly in consumables, for instance, a monosaccharide composition such as glucose, fructose, etc, a disaccharide composition such as sucrose or an artificial sugar composition. The control composition may be table sugar, corn syrup, high-fructose corn syrup, or any other suitable composition. The term “comparable,” as used herein, generally means that the two compositions may be up to 100%, up to 95%, up to 90%, or up to 80% identical. For instance, comparable can mean that the composition is up to 90% identical to the control composition.

In some cases, the compositions described herein may be used as sweetener compositions. Sweetener compositions may be used by themselves or as an ingredient in a finished product. The compositions described herein may provide about the same level of sweetness or greater sweetness than an identical amount of a control composition wherein the control composition comprises primarily monosaccharides and/or disaccharides. The compositions described herein may be used to replace the control composition as the sweetener in a finished product. In some cases, the sweetness of a composition may be 5%, 10%, 15%, 20%, 30%, 40%, 50%, 70%, 80%, 90%, or 100% more than an identical amount of the control composition.

The compositions described herein may provide a comparable flavor profile or better flavor profile than an identical amount of a control composition wherein the control composition comprises primarily monosaccharides and/or disaccharides. The compositions described herein may be used to replace the control composition as a flavor enhancer in a finished product. In some cases, the flavor of a composition may be 5%, 10%, 15%, 20%, 30%, 40%, 50%, 70%, 80%, 90%, or 100% more than an identical amount of the control composition.

The compositions described herein may provide a comparable texture profile or better texture profile than an identical amount of a control composition wherein the control composition comprises primarily monosaccharides and/or disaccharides. The compositions described herein may be used to replace the control composition as a texture enhancer in a finished product.

The compositions described herein may provide a comparable binding profile or better binding profile than an identical amount of a control composition wherein the control composition comprises primarily monosaccharides and/or disaccharides. The compositions described herein may be used to replace the control composition as a binding enhancer in a finished product.

The compositions described herein may provide a comparable glazing profile or better glazing profile than an identical amount of a control composition wherein the control composition comprises primarily monosaccharides and/or disaccharides. The compositions described herein may be used to replace the control composition as a glazing enhancer in a finished product.

The compositions described herein may provide a comparable moistness or better moistness than an identical amount of a control composition wherein the control composition comprises primarily monosaccharides and/or disaccharides. The compositions described herein may be used to replace the control composition to provide moistness in a finished product.

The compositions described herein may provide a comparable color profile or better color profile than an identical amount of a control composition wherein the control composition comprises primarily monosaccharides and/or disaccharides. The compositions described herein may be used to replace the control composition as a color enhancer in a finished product.

The compositions described herein may provide a comparable dissolution profile or better dissolution profile than an identical amount of a control composition wherein the control composition comprises primarily monosaccharides and/or disaccharides. The compositions described herein may be used to replace the control composition as a dissolution enhancer in a finished product. In some cases, the dissolution of a composition may be 5%, 10%, 15%, 20%, 30%, 40%, 50%, 70%, 80%, 90%, or 100% more than an identical amount of the control composition.

The compositions described herein may provide a comparable mouthfeel or better mouthfeel than an identical amount of a control composition wherein the control composition comprises primarily monosaccharides and/or disaccharides.

The compositions described herein may provide a comparable viscosity or better viscosity than an identical amount of a control composition wherein the control composition comprises primarily monosaccharides and/or disaccharides.

The compositions described herein may provide a comparable hygroscopicity or better hygroscopicity than an identical amount of a control composition wherein the control composition comprises primarily monosaccharides and/or disaccharides. In some cases, the hygroscopicity of a composition may be 5%, 10%, 15%, 20%, 30%, 40%, 50%, 70%, 80%, 90%, or 100% more than an identical amount of the control composition.

The compositions described herein may provide a comparable water-retention or better water-retention than an identical amount of a control composition wherein the control composition comprises primarily monosaccharides and/or disaccharides. In some cases, the water-retention of a composition may be 5%, 10%, 15%, 20%, 30%, 40%, 50%, 70%, 80%, 90%, or 100% more than an identical amount of the control composition.

The compositions described herein may provide a lower calorie composition than an identical amount of a control composition wherein the control composition comprises primarily monosaccharides and/or disaccharides. In some cases, the calorie count of a composition may be 5%, 10%, 15%, 20%, 30%, 40%, 50%, 70%, 80%, 90%, or 100% less than an identical amount of the control composition.

The compositions described herein may provide a lower glycemic index than an identical amount of a control composition wherein the control composition comprises primarily monosaccharides and/or disaccharides. In some cases, the glycemic index of a composition may be 5%, 10%, 15%, 20%, 30%, 40%, 50%, 70%, 80%, 90%, or 100% less than an identical amount of the control composition.

The compositions described herein may provide a comparable bulking or better bulking than an identical amount of a control composition wherein the control composition comprises primarily monosaccharides and/or disaccharides.

The compositions described herein may provide a comparable caramelization or better caramelization than an identical amount of a control composition wherein the control composition comprises primarily monosaccharides and/or disaccharides.

The compositions described herein may provide a comparable surface texture or better surface texture than an identical amount of a control composition wherein the control composition comprises primarily monosaccharides and/or disaccharides.

The compositions described herein may provide a comparable crystallization or better crystallization than an identical amount of a control composition wherein the control composition comprises primarily monosaccharides and/or disaccharides.

The compositions described herein may provide comparable structural properties as an identical amount of a control composition wherein the control composition comprises primarily monosaccharides and/or disaccharides.

The compositions described herein may provide less aftertaste compared to an identical amount of a control composition wherein the control composition comprises primarily monosaccharides and/or disaccharides.

Different compositions of oligosaccharides may have improved dissolution profiles, hygroscopicity profiles, and taste profiles compared to the oligosaccharides used alone.

The compositions or ingredients as described herein may be used to increase the fiber content of a finished product such as a foodstuff or a nutraceutical. The compositions may provide a higher level of fiber in the finished product as compared to an identical amount of a control composition wherein the control composition comprises primarily monosaccharides and/or disaccharides. In some cases, the compositions may improve the fiber content of the finished product without negatively, or substantially negatively, affecting any other properties such as taste, sweetness, mouthfeel, texture, binding, or any other properties described herein. In some cases, the fiber content of a composition may be 5%, 10%, 15%, 20%, 30%, 40%, 50%, 70%, 80%, 90%, or 100% more than an identical amount of the control composition.

Ingredients may be used to alter the properties of a finished product such as foodstuff or nutraceutical or cosmetic. In order to alter the properties of the finished products, the finished products may additionally comprise a polysaccharide, for example, a cellulosic polysaccharide, such as cellulose, or a polysaccharide derivative, for example, a cellulose derivative, such as carboxymethylcellulose, or a polysaccharide aggregate, for example, a portion of lignocellulosic biomass. In some instances, the finished products can comprise from greater than 0% to 40% by dry weight of polysaccharide, polysaccharide derivative, or polysaccharide aggregate, for example, from greater than 1% to 30% by dry weight of polysaccharide, polysaccharide derivative, or polysaccharide aggregate, for example, from greater than 5% to 25% by dry weight of polysaccharide, polysaccharide derivative, or polysaccharide aggregate, for example, from greater than 10% to 20% by dry weight of polysaccharide, polysaccharide derivative, or polysaccharide aggregate.

The concentration of a composition comprising polysaccharides and a mixture of oligosaccharides in a finished product may be anywhere from 0.1% to 40% w/w. The concentration of a composition comprising polysaccharides and a mixture of oligosaccharides in a finished product may be from about 0.1% to about 0.5%, about 0.1% to about 1%, about 0.1% to about 5%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.5% to about 1%, about 0.5% to about 5%, about 0.5% to about 10%, about 0.5% to about 15%, about 0.5% to about 20%, about 0.5% to about 25%, about 0.5% to about 30%, about 0.5% to about 35%, about 0.5% to about 40%, about 1% to about 5%, about 1% to about 10%, about 1% to about 15%, about 1% to about 20%, about 1% to about 25%, about 1% to about 30%, about 1% to about 35%, about 1% to about 40%, about 5% to about 10%, about 5% to about 15%, about 5% to about 20%, about 5% to about 25%, about 5% to about 30%, about 5% to about 35%, about 5% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% w/w. The concentration of a composition comprising polysaccharides and a mixture of oligosaccharides in a finished product may be about 0.1%, about 0.5%, about 1%, about 5%, about 10%, about 15%, about 20%, about 25%, about 30%, about 35%, or about 40% w/w. The concentration of a composition comprising polysaccharides and a mixture of oligosaccharides in a finished product may be at least 0.1%, 0.5%, 1%, 5%, 10%, 15%, 20%, 25%, 30%, or 35% w/w. The concentration of a composition comprising polysaccharides and a mixture of oligosaccharides in a finished product may be at most 0.5%, 1%, 5%, 10%, 15%, 20%, 25%, 30%, 35%, or 40% w/w.

In some cases, the oligosaccharide mixtures (e.g., cello-oligosaccharides and xylo-oligosaccharides) may form at least 20%, 30%, 40%, 50%, 60%, or 70% w/w of the consumable composition or ingredient. For example, 50% w/w of a combination of cello-oligosaccharides and xylo-oligosaccharides may form the consumable composition or ingredient.

In some instances, the ingredient may include a maize cob extract (MCE). The MCE may be a mixture of oligosaccharides composed primarily of arabinoxylo-oligosaccharides, xylo-oligosaccharides, cello-oligosaccharides, and cellulose. In certain instances, the oligosaccharides may be non-digestible or substantially non-digestible. The arabinoxylo-oligosaccharides can be oligomers with xylose backbones linked by β-(1→4)-bonds substituted with arabinose side-chains. The arabinoxylo-oligosaccharides may be non-digestible. The arabinoxylo-oligosaccharides can be produced by hydrolysis of arabinoxylan (polysaccharide of β-(1→4)-bonded xylose units substituted with arabinose side-chains). Furthermore, the arabinoxylo-oligosaccharides may have a degree of polymerization (DP) of 3 to 15.

In various instances, the xylo-oligosaccharides may be oligomers with xylose backbones linked by β-(1→4)-bonds. The xylo-oligosaccharides may be non-digestible. The xylo-oligosaccharides may be produced by hydrolysis of arabinoxylan. Moreover, the xylo-oligosaccharides may have a DP of 2 to 8.

In certain instances, the cello-oligosaccharides may be oligomers with glucose backbones linked by β-(1→4)-bonds. The cello-oligosaccharides may be non-digestible. The cello-oligosaccharides may be produced by hydrolysis of cellulose (polysaccharide of β-(1→4)-bonded glucose units). Furthermore, the cello-oligosaccharides may have a DP of 2 to 4, with the majority having a DP of 2.

### IV. Exemplary Embodiments

Exemplary Method of Extracting Soluble Polysaccharides for Subsequent Combination with Generated Oligosaccharides

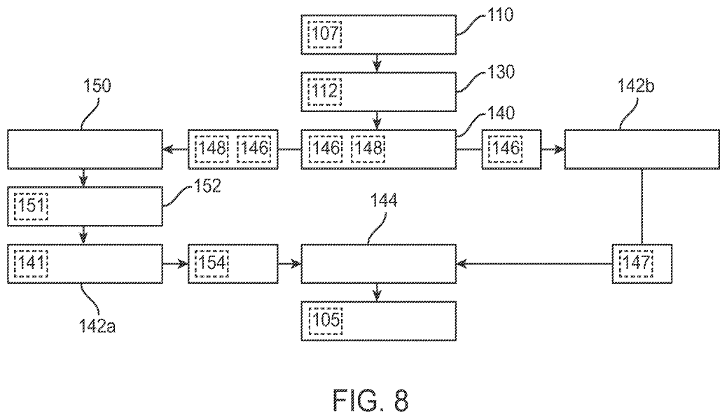

FIG. 8 is a simplified flow diagram showing an embodiment of a method of extracting soluble polysaccharides for subsequent combination with generated oligosaccharides to form an ingredient.

In the illustrated method, the extraction or removal of at least a portion of the soluble polysaccharides 140 before an enzyme treatment 150 can ensure that at least a portion of the soluble polysaccharides are retained or saved for combination 144 with the generated oligosaccharides to form the ingredient 105. In some other methods (not shown), the soluble polysaccharides can be digested by the one or more enzymes because the soluble polysaccharides are not extracted prior to the enzyme treatment. The ingredient 105 of the illustrated method can be a sweetener or sugar substitute that can remain substantially soluble or entirely soluble. Accordingly, the ingredient 105 can be delivered as a syrup-like product (e.g., a viscous liquid). In some cases, the ingredient 105 can be a replacement or partial replacement for corn syrup, high-fructose corn syrup, maple syrup, honey, treacle, golden syrup, molasses, dextrose syrup, fructose syrup, agave nectar, date syrup, brown rice syrup, coconut syrup, corn syrup or other suitable liquid sweeteners in a foodstuff.

As depicted, biomass 107 (e.g., corncob or any other suitable biomass) can be physically pretreated 110 (e.g., by chipping or any other suitable method of physically pretreating a biomass). The physically treated biomass 112 can then be subjected to or undergo a thermochemical pretreatment 130 (e.g., 15% w/v chipped corncob can be heated in 1% w/v NaOH for one hour). In some embodiments, the thermochemical pretreatment 130 can be followed by a neutralization step (not shown) before extraction of the soluble compounds or material 140 from the physically pretreated biomass 112. Extraction of the soluble compounds or material 140 can include removing a liquid portion (e.g., a supernatant) of the physically pretreated biomass 112. The liquid portion can include soluble compounds 146 from the physically pretreated biomass 112. The soluble compounds 146 can include soluble polysaccharides. In certain embodiments, 15% of the liquid portion (including the soluble compounds or material 146) can then be extracted or removed from the thermochemically pretreated biomass 112. The extracted portion including the soluble compounds or material 146 (e.g., the soluble polysaccharides) can then be subjected to one or more purification steps 142b (e.g., ultrafiltration) to enrich the soluble polysaccharides 147.

Furthermore, the liquid portion that is not extracted at step 140 can include soluble compounds or material 146 and insoluble compounds or material 148. For example, in the case where 15% of the liquid portion is extracted as described above, the remaining portion of the physically pretreated biomass 112 (including 85% of the liquid portion) can include soluble polysaccharides and insoluble polysaccharides. The solution including the soluble compounds or material 146 and insoluble compounds or material 148 can then be subjected to or undergo an enzyme treatment 150, as disclosed herein. For example, one or more polysaccharide-cleaving enzymes can be added to the solution including the soluble compounds or material 146 and insoluble compounds or material 148 to 0.5% w/v and incubated at 50° C. for 24 hours. The enzyme-treated biomass 151 can then be treated (e.g., filtered) 152 to remove at least a portion of the undigested biomass. In certain cases, the removed undigested biomass can be disposed of or rejected. The digested biomass 141 can then be subjected to or undergo purification 142a (e.g., ion-exchange chromatography, nanofiltration, microfiltration, ultrafiltration, or any other suitable method of purification) to enrich for oligosaccharides 154 as described herein. The extracted, isolated, and/or purified oligosaccharides 154 and the extracted, isolated, and/or purified soluble polysaccharides 147 can be combined, mixed, and or spray dried to form the ingredient 105.

Exemplary Method of Pretreating Biomass to Remove Monosaccharides and/or Disaccharides

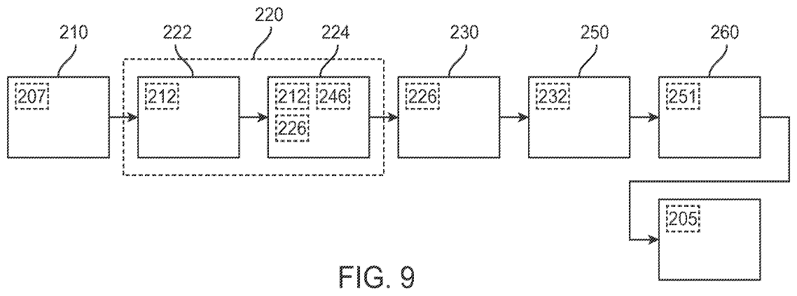

FIG. 9 is a simplified flow diagram showing an embodiment of a method of pretreating biomass to remove monosaccharides and/or disaccharides prior to an enzyme treatment.

The embodiment of FIG. 9 may include components or steps that resemble the components or steps of the embodiment of FIG. 8 in some respects. For example, the embodiment of FIG. 9 includes the step of physically pretreatment 110 that may resemble the physical pretreatment 210 of FIG. 8. It will be appreciated that the illustrated embodiments may have analogous features. Accordingly, like features are designated with like reference numerals, with leading digits added to increment each reference numeral by 100. For instance, the physical pretreatment is designated “110” in FIG. 8 and an analogous physical pretreatment is designated as “210” in FIG. 9. Relevant disclosure set forth above regarding similarly identified features thus may not be repeated hereafter. Moreover, specific features of the method and related components or steps shown in FIG. 9 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the method and related components or steps of FIG. 9. Any suitable combination of the features, and variations of the same, described with respect to the method illustrated in FIG. 8, can be employed with the method and components or steps of FIG. 9, and vice versa. This pattern of disclosure applies equally to further embodiments depicted in subsequent figures and/or described hereafter.

As illustrated, a gentle pretreatment 220 (e.g., a washing or incubation cycle as provided herein) of a physically pretreated biomass 212 can include removing 224 the soluble compounds 246. In certain cases, the soluble compounds 246 can include monosaccharides and/or disaccharides. The removed soluble monosaccharides and/or disaccharides can then be discarding and/or rejected. Accordingly, the gentle pretreatment 220 can be conducted or performed to remove soluble monosaccharides and/or disaccharides from the biomass 207.

Biomass 207 can be physically pretreated 210 (e.g., chipped) and then the physically pretreated biomass 212 can be subjected to or undergo a gentle pretreatment 220, for example, washed or incubated (e.g., in water at 25° C. for 30 minutes). Soluble saccharides 246 (e.g., soluble monosaccharides and/or disaccharides) can then be removed from the solution including the gently pretreated biomass 226. The gently pretreated biomass 226 can then be subjected to or undergo a strong pretreatment 230. In certain cases, the strong pretreatment 230 can be a thermochemical pretreatment. For example, the gently pretreated biomass 226 can be treated in 1% w/v NaOH at 100° C. for 60 minutes. In some embodiments, the strong pretreatment 230 can be followed by a neutralization step (not shown) before the enzyme treatment 250. The strongly pretreated biomass 232 can then be treated with enzymes 250 as discussed herein. Furthermore, downstream processing 260 can then be conducted on the enzyme-treated biomass 251 to generate the ingredient 205.

Additional Exemplary Embodiments of Extracting Soluble Polysaccharides for Combination with Oligosaccharides

In some instances, the disclosure relates to novel methods of treating plant biomass materials for the production of a foodstuff, cosmetic, or nutraceutical ingredient.

Sugary foods and drinks are an important part of culture and lifestyle habits across the world, but the sugar they contain has been linked to obesity, diabetes, poor dental health, and disruptive behavior in people. Because of this, consumer preferences have been shifting away from sugar-containing foods, and governments are increasingly implementing regulation to encourage the consumption of less sugar.

As such, industry has been searching for suitable low-calorie sweeteners for many decades to substitute for sugar in food and beverages. Unfortunately, many sugar substitutes are produced from non-natural resources, and often offer bitter undertones or other unpleasant tastes along with their sweetness, both of which consumers find unappealing. Moreover, while many sweeteners are able to mimic the sweetness of sugar in food and drinks, few are able to mimic the broad range of roles that sugar plays in food, such as adding bulk, modulating texture, providing structure, acting as a preservative, and modulating color and flavor through caramelization and Maillard reactions.

Dietary fiber is an important part of a positive diet, and helps maintain digestive health and a well-regulated gut flora. Such fiber comprises saccharides of varying chain lengths and types. In addition to being found naturally in a wide spectrum of foods, fiber can also be produced separately and added to other foods during their manufacture.

Biomass is a good source of saccharides that can be used to replace sugar and add fiber to food products. However, there remains a need to optimize the process by which these saccharides are obtained from the biomass and processed into the compositions useful as a foodstuff, cosmetic, or nutraceutical ingredient.

Firstly, the saccharides generally need to be produced by controlled breakdown. Different amounts of different sized saccharides in the ingredient can affect its nutritional values, and also other properties such as hygroscopicity which in turn affect properties such as the texture of the product the ingredient is used to make. It can also be desirable for the ingredient to comprise polysaccharide as polysaccharide can improve gastrointestinal tolerance. However, due to the speed at which some particularly desirable polysaccharides break down during the enzymatic reaction of previously known methods, it is difficult to isolate and then incorporate into the ingredient these desirable polysaccharides.

Furthermore, food products that require a smooth texture, such as candy, chocolate, and yoghurt, generally require the ingredient to be soluble to achieve the smooth texture. Ingredients comprising insoluble polymeric material can result in a gritty texture. However, due to the insolubility of certain polysaccharides, it can be difficult to make a composition comprising polysaccharides that is entirely soluble, particularly in a one-pot process from a single piece of biomass. Typically, soluble polysaccharides break down quicker than insoluble polysaccharides so after exposing a plant biomass to enzymes, like of previously known methods, the soluble polysaccharides are entirely broken down and only insoluble polysaccharides are left.

Surprisingly, methods have been identified here that can allow polysaccharides to be isolated and incorporated into a soluble foodstuff, cosmetic, or nutraceutical ingredient comprising oligosaccharides, which thus maintains the benefit of increasing the gastrointestinal tolerance of the ingredient as well as allowing it to be used in food products with a smooth texture. The polysaccharides can be isolated from the same plant biomass as that which the other desired saccharides are obtained from providing an efficient and stream-lined production process.

Accordingly, in a first aspect of the disclosure there is provided a method for producing a foodstuff, cosmetic, or nutraceutical ingredient comprising the steps of:
a) providing a plant biomass comprising one or more soluble polysaccharides and one or more insoluble polysaccharides;
b) treating the plant biomass to dissolve the one or more soluble polysaccharides;
c) removing a portion of the dissolved one or more soluble polysaccharides;
d) reacting the remaining plant biomass with one or more enzymes to form one or more oligosaccharides;
e) removing the one or more oligosaccharides; and
f) combining the portion of the dissolved one or more soluble polysaccharides from step (c) and the one or more oligosaccharides from step (e) to form the ingredient.

As such, there is also provided a foodstuff, cosmetic, or nutraceutical ingredient obtainable by the methods of the disclosure.

In another aspect of the disclosure there is provided a foodstuff, cosmetic, or nutraceutical liquid ingredient comprising at least one oligosaccharide selected from the list consisting of:
i) cello-oligosaccharide having a degree of polymerization of from two to six;
ii) xylo-oligosaccharide having a degree of polymerization of from two to twelve;
iii) manno-oligosaccharide having a degree of polymerization of from two to twelve;
iv) mixed-linkage glucan oligosaccharide having a degree of polymerization of from two to five;
v) xyloglucan oligosaccharide having a degree of polymerization of from four to twelve; and
vi) chito-oligosaccharide having a degree of polymerization of from two to twelve;
and at least one polysaccharide selected from the list consisting of:
i) xylan;
ii) mannan;
iii) cellulose derivative;
iv) mixed-linkage glucan;
v) xyloglucan; and
vi) chitosan;
wherein the liquid ingredient comprises at least 20% by dry weight of the at least one oligosaccharide and at least 2% by dry weight of the at least one polysaccharide and wherein the liquid ingredient has a viscosity of from 5 to 100,000 cps.

Preparing the foodstuff, cosmetic, or nutraceutical ingredient in the manner provided herein can allow efficient use of biomass by incorporating oligomeric and polymeric material from the same biomass source to make a soluble ingredient. Furthermore, the methods can allow for purification, derivatization, or other modification, as well as control of oligomeric and polymeric proportions, which can improve the functional properties, nutritional properties, and tolerance of the ingredient.

Any substance which comprises suitable polysaccharides may be the plant biomass. As the foodstuff, cosmetic, and nutraceutical industries use a broad variety of oligosaccharides, the polysaccharides suitable in the method are not particularly limited. Plant biomass suitable for producing the oligosaccharide profile of the current disclosure may comprise, for example, cellulose, lignocellulose, chitin, chitosan, xylan (such as glucuronoxylan, arabinoxylan, and glucuronoarabinoxylan), xyloglucan, mixed-linkage glucan, and/or mannan (such as glucomannan, galactomannan, or galactoglucomannan), however, any plant biomass which can be suitably acted upon is envisaged. The one or more soluble polysaccharides that the plant biomass comprises may include any one of the following: mannans, mixed-linkage glucans, lignocellulose, hemicellulose, certain cellulose derivatives such as cellulose acetate, hydroxyethylcellulose, and hydroxymethylcellulose, and chitosan. In some embodiments, the plant biomass comprises hemicellulose. In certain embodiments, the hemicellulose comprises xylan and/or mannan.

As such, the plant biomass may be grain, grain chaff, bean pods, seed coats, and/or other seed materials; seaweeds; corn stover, straw, bagasse, miscanthus, sorghum residue, switch grass, bamboo, and/or other monocotyledonous tissue; water hyacinth, leaf tissue, roots, and/or other vegetative matter; and/or any combination of suitable plant biomasses. In some cases, the plant biomass comprises, or suitably consists of, sugar cane biomass (such as sugar cane bagasse), corn biomass (such as corncob or corn stover), wheat biomass (such as wheat straw or wheat bran), hardwood or softwood. In certain cases, the plant biomass comprises corncob, sugar cane bagasse, wheat straw, or rice straw.

In various instances, in step (b) the “treating” is a thermochemical treatment of the plant biomass. “Thermochemical,” as used herein, generally refers to heating above room temperature (room temperature can be about 20° C. to 22° C.) the plant biomass in a chemical substance, such as heating in a solution comprising water, alkali, or ionic solvents. The thermochemical step can physically and chemically modify chemical components of the plant biomass. For example, the free hydroxide ions from the water or alkali can disrupt hydrogen bonds between saccharides enabling the solubilization of some types of saccharides, for example, hemicelluloses, and can better enable the enzyme in the subsequent step to more easily break up the saccharides. These disrupted hydrogen bonds may be between monomers of the same saccharide chain which contribute to the chain's tertiary structure. The disrupted hydrogen bonds may also be between monomers of different saccharide chains which contribute to the quaternary structure of more than one chain. Subsequently, the treatment can result in the one or more polysaccharides that are soluble (i.e., the polysaccharides that are particularly susceptible to the disruption of the hydroxide ions, especially, for example, hemicelluloses) to be dissolved into the chemical substance used. The one or more polysaccharides that are insoluble, such as cellulose, do not dissolve into the chemical substance.

The heating of the treatment step (b) may be at a range of temperatures, suitably of from 30° C. to 180° C., 50° C. to 150° C., or from 70° C. to 120° C. Higher temperatures can help the soluble polysaccharides to dissolve quicker, however, temperatures that are too high can be more difficult to achieve in an efficient and cost-effective manner, and can chemically modify the biomass components, including the saccharides (e.g., in an undesirable manner).

The heating may occur for a range of time scales, particularly large amounts of biomass may be exposed to heating for a longer period of time, which can be adjusted accordingly. For example, the heating of the plant biomass can be of from 1 minute to 72 hours, 10 minutes to 24 hours, 20 minutes to 12 hours, or 25 minutes to 8 hours.

In some instances, the thermochemical treatment may comprise heating the plant biomass in water, i.e., at a neutral pH of about pH 7.

In certain instances, the thermochemical treatment may comprise heating the plant biomass in an alkali solution having a pH of from 8 to 14, 9 to 14, or 10 to 14. The solution may comprise, or suitably consist of, any one of the alkalis selected from: sodium hydroxide, potassium hydroxide, sodium carbonate, calcium carbonate, calcium hydroxide, ammonium sulfate, ammonium hydroxide, and aqueous ammonia. In various instances, the alkali may be sodium hydroxide. A combination of the listed alkalis is also envisaged.

Multiple different sequential thermochemical treatment steps are also envisaged. For example, there may be two sequential thermochemical treatments, there may be three sequential thermochemical treatments, or there may be four or more sequential thermochemical treatments. In some cases, the biomass may be thermochemically treated in a neutral aqueous solution and then thermochemically treated in an alkaline aqueous solution.

After the treating step, step (c) can comprise removing a portion of the dissolved one or more soluble polysaccharides. The purpose of this step can be to isolate and remove the soluble polysaccharides from the plant biomass so that they do not get broken down and therefore lost in the subsequent enzymatic reaction. This can enable the use of these polysaccharides when forming the ingredient in a later step. All of the dissolved polysaccharides or a portion of them may be removed dependent on the desired amount in the final ingredient. The soluble polysaccharides can be removed using simple steps such as filtering the chemical substance, of which the soluble polysaccharides are dissolved in.

Step d) comprises reacting the remaining plant biomass, which may be in the form of a solution and/or a suspension, with one or more enzymes to form the one or more oligosaccharides. The soluble and insoluble polysaccharides present in the remaining plant biomass solution and/or suspension can be partially or fully cleaved by the one or more enzymes into oligosaccharides (e.g., useful oligosaccharides), potentially leaving partially cleaved, or uncleaved, polysaccharides, which may include cellulose, xylan (such as glucuronoxylan, arabinoxylan, or glucuronoarabinoxylan), mannan (such as glucomannan, galactomannan, or galactoglucomannan), mixed-linkage glucan, xyloglucan, chitin, chitosan, or lignocellulose.

The enzyme reaction may take place in solution and/or suspension, in a suitable reaction vessel. The enzyme reaction may take place at a temperature or temperature protocol suitable for the particular combination of enzyme and plant biomass, the reaction may be allowed to progress for a certain amount of time, until the products have reached a desired concentration, or until some other requirement has been met.

In order to ensure optimal contact between the enzymes and the plant biomass, the reaction mixture may be agitated, either constantly or at intervals. The agitation may take the form of rhythmically moving the entire reaction vessel, of a fan or other stirring device, of a bubble sparging, or any other method of agitation.

The enzymatic reaction may be a microbial fermentation. The temperature and reaction time can be suitable for the growth of the microbial organism used. The microbial organism may be genetically altered to produce an enzyme suitable for the production of an oligosaccharide of the present disclosure. The microbe may be, for example, a bacterium, for example, Escherichia coli, or a fungus, such as Saccharomyces cerevisiae, Aspergillus niger, or Trichoderma reesei.

Further embodied in the present disclosure is an expression vector suitable for modifying the subject microorganism such that it produces an enzyme or mixture of enzymes of the current disclosure. Where desired, the expression vector, which may be a plasmid or any other nucleic acid able to induce production of the enzyme, may comprise one or more of the following regulatory sequences so as to control the expression of the exogenous enzyme: regulatory sequences of a heat shock gene, regulatory sequences of a toxicity gene, and regulatory sequences of a spore formation gene.

The enzymatic reaction can be carried out at a temperature or temperature protocol suitable to the enzymes and substrates used. For example, it may be carried out at a constant temperature in the range of from about 10° C. to about 100° C., about 20° C. to about 70° C., or about 30° C. to about 60° C. If the enzymatic reaction takes the form of a microbial fermentation the temperature may be suitable for such, for example, the enzymatic reaction may comprise the growth of E. coli and/or the temperature may be constant and about 37° C.

The pH of the solution or suspension may affect the activity of the enzymes. Control of pH may assure that an enzymatic reaction proceeds at a suitable rate. The enzymatic reaction of the present disclosure may take place at a pH in the range of from about 2 to about 10, about 3 to about 8, or about 4 to about 6.

The enzymatic reaction can be allowed to continue for a certain time period before being quenched, and the products isolated or otherwise collected. This time period may be from about 1 minute to about 6 days, from about 0.5 days to about 5 days, or from about 16 hours to about 96 hours. The reaction may alternatively be allowed to proceed until no further catalysis occurs.

The enzymatic reaction can be allowed to continue to run until there is less than 75% undigested polysaccharide-containing plant biomasses remaining, less than 70%, less than 65%, less than 55%, or less than 50%. This can be monitored or checked by reducing end assays, such as the anthrone assay and/or by chromatographic methods such as thin-layer chromatography and high-performance anion exchange chromatography. The reaction may run until all polysaccharides are converted to oligosaccharides.

There are many enzymes that are suitable for use in the enzymatic reaction of the present method. For example, “lytic polysaccharide monooxygenase” and “LPMO,” which are a class of enzymes able to oxidatively cleave polysaccharides using a copper comprising moiety and using an oxygen source, such as a molecule of dioxygen, peroxide, or any other oxygen source; and a suitable reducing agent. As such, when an LPMO is used, the enzymatic reaction may be carried out under aerobic conditions. Suitable reducing agents are not particularly limited, but examples include ascorbic acid, gallic acid, cysteine, NADH, NADPH, pyrogallol, dithiothreitol, cyanoborohydrides, borohydrides, photosynthetic pigments, lignin, lignols, and a combination of cellobiose and cellobiose dehydrogenase. A wide variety of photosynthetic pigments may be used. In some embodiments, thylakoids and purified fractions, or chlorophyllin may be used, and light may be supplied. LPMOs can be selected from the following families: AA9, AA10, AA11, AA13, AA14, and AA15. In various cases, the LPMO may be PaLPMO9E (SEQ ID NO:1), an AA9 LPMO originally isolated from the ascomycete fungus Podospora anserina or the LPMO may be an AA9 LPMO from Trichoderma reesei (SEQ ID NO:23).

Aerobic conditions may comprise the addition of oxygen, which may be provided by aeration of the substrate mixture with an oxygen-comprising gas, such as air. Aeration may be conducted by the introduction of oxygen-comprising air bubbles into the aqueous substrate mixtures by various systems, such as an air-injector, an aeration frit, a membrane system, or an internal-loop airlift reactor. The concentration of molecular oxygen in the enzymatic reaction may be from about 4 mg/L to about 14 mg/L.

Another type of enzyme that can be used in the method is a “cellulase,” which has hydrolytic activity against cellulose, for example, endo-1,4-beta-glucanase, cellobiohydrolase, and/or beta-glucosidase activities. Such enzymes are able to cleave glycosidic bonds in one or more forms of cellulose, including cellulose found in plant biomass. In doing so, they produce products including glucose and cello-oligosaccharides. In certain cases, the beta-glucanases may include enzymes from GH5, GH7, and GH12 enzyme, such as those derived from Aspergillus niger (SEQ ID NO:12, 13 and 14) and Trichoderma reesei (SEQ ID NOs: 24 and 25).

Another type of enzyme is “cellobiohydrolase,” which has hydrolytic activity against cellulose and produces mainly cellobiose as a product. Cellobiose is a disaccharide and is a cello-oligosaccharide. Such enzymes are able to cleave glycosidic bonds in one or more forms of cellulose, including cellulose found in plant biomass. In various instances, cellobiohydrolases may be from GH6 and GH7 enzyme families, Cel6A or Cel7A enzymes derived from Trichoderma reesei (SEQ ID NOs: 10 and 11, respectively).

Another type of enzyme is “beta-glucosidase,” which has hydrolytic activity against cellulose and produces mainly glucose as a product. Such enzymes are able to cleave glycosidic bonds in one or more forms of cellulose, including cellulose found in plant biomass. In some embodiments, beta-glucosidases may include GH3 beta-glucosidases, such as one from Trichoderma reesei (SEQ ID NO: 22).

Another type of enzyme is a lichenase, which may be selected from: the GH5, GH7, GH8, GH9, GH12, GH16, GH17, or GH26 families. In some embodiments, the lichenase may be a GH16 enzyme, for example, a GH16 enzyme derived from Bacillus subtilis (SEQ ID NO:2). The enzyme is able to act on, for example, mixed-linkage glucans, which are glucans comprising a mixture of β-1,3 and β-1,4 linkages, and may cleave them at β-1,4 glycosidic bonds. In the case in which the lichenase acts on a mixed-linkage glucan, the β-glucans produced may fall largely within the size range of from about 3 to about 7 residues, so they are particularly useful in the food, cosmetics, and nutraceutical industries. Mixed-linkage glucans are abundant in members of the grass and horsetail families, and as such, grass-based biomasses such as straw have high levels of mixed-linkage glucans, and may be acted upon usefully with lichenases.

Another type of enzyme is a xylanase, which may act on, for example, plant biomass comprising a xylan backbone. The xylanase may be, for example, a glucuronoxylanase, an arabinoxylanase, or a glucuronoarabinoxylanase. The enzyme may be active on a variety of polymers having a xylan backbone, such as glucuronoxylan, arabinoxylan, and glucuronoarabinoxylan. These polymers are abundant in various plant biomass, for example, both hardwood and softwood may comprise suitable polysaccharides, with hardwood often comprising glucuronoxylan and softwood often arabinoglucuronoxylan. In some instances, xylanases may include GH5 xylanases from Ruminiclostridium thermocellum (SEQ ID NO:3) and Gonapodya prolifera (SEQ ID NO:4), and GH30 xylanases from Dickeya chrysanthemi (SEQ ID NO:5), Bacillus subtilis (SEQ ID NO:6), Bacteroides ovatus (SEQ ID NO:7), and Trichoderma reesei (SEQ ID NO: 15).

Other enzymes useful in the disclosure may include xyloglucanases and xyloglucan endoglucanases (XEGs), which are produced by numerous organisms, including plant-pathogenic microbes. They are able to act on xyloglucan, a hemicellulosic β-1,4 glucan chain abundant in the primary cell wall of higher plants, which is decorated with xylose, some of the xylose residues being further decorated with other residues, such as galactose. When suitable xyloglucanases or XEGs act on xyloglucan, the products comprise xyloglucan oligosaccharides having a main chain of a length useful in the foodstuff, cosmetics, and nutraceutical industries. In some cases, xyloglucanases may include a GH5 xyloglucanase from Bacteroides ovatus (SEQ ID NO:8) and a GH74 xyloglucanase from Trichoderma reesei.

As any given natural plant biomass is likely to comprise a mixture of different polysaccharides, sometimes it may be the case that a mixture of different enzymes is beneficial. Such a mixture may comprise one or more of any other enzyme. For example, such a mixture might comprise an LPMO with an endo-glucanase, a xylanase with a lichenase, a cellobiohydrolase with a mannanase, or an endo-glucanase with a cellobiohydrolase in which the enzyme partners are present in molar ratios, for example, from 1:100 to 100:1.

In certain cases, the one or more enzymes may be a cocktail of different enzymes, for example, a crude or semi-crude enzyme preparation. The term “crude enzyme preparation” as used herein generally refers to a soluble preparation extracted from a microbial fermentation that has undergone minimal processing after the extraction, for example, typically the preparation may only undergo filtration in order to remove insoluble components. The term “semi-crude enzyme preparation” as used herein generally refers to a soluble preparation extracted from a microbial fermentation that has undergone some processing after the extraction, for example, the preparation may undergo filtration in order to remove insoluble components, increasing the enzyme concentration and/or nanofiltration to remove small molecular weight compounds.

In certain cases, the crude or semi-crude enzyme preparation may be from a bacteria or a fungus. In some embodiments, the crude or semi-crude enzyme preparation may be from a fungus, such as a filamentous cellulolytic fungus, such as from Trichoderma or Aspergillus species. In certain embodiments, the enzyme may be a crude or semi-crude enzyme preparation from a Trichoderma reesei strain.

In step (e), the one or more oligosaccharides formed in step (d) are removed, which may be done in a number of ways. They may be isolated based on solubility, so that a composition of soluble saccharides only is extracted for further processing, and/or isolated chromatographically to produce a composition with a narrower band of oligosaccharide chain lengths. Isolation may, for example, be based on precipitation, size-exclusion chromatography, ion-exchange chromatography, filtration, ultrafiltration, microfiltration, or nanofiltration. In the case that isolation based on solubility is carried out, the profile of saccharides present in the isolated composition may depend on the original enzymatic reaction, as different saccharides decrease in solubility with length at different rates.

Also envisaged in the scope of the present disclosure is the further treatment of all or part of the removed one or more oligosaccharides to produce further products before combining them with the one or more dissolved polysaccharides to form the ingredient. This further treatment may comprise any chemical, physical, or enzymatic step, such as reduction, for example, reductive amination where suitable; oxidation, caramelization, modification with a Schiff base, or via the Maillard reaction, or by any combination of such steps, and may provide different products having properties which are improved for the desired purpose. For example, the caramelization properties, calorific value, flavor, and color may be modified. The oligosaccharides may also be purified, for example, through precipitation, size-exclusion chromatography, ion-exchange chromatography, filtration, ultrafiltration, microfiltration, or nanofiltration.

Also envisaged in the scope of the disclosure is the further treatment of all or part of the dissolved one or more soluble polysaccharides to produce products with improved properties before combining with the one or more removed oligosaccharides to form the ingredient. This further treatment may comprise any chemical, physical, or enzymatic step, such as alkylation or acid-treatment. The polysaccharides may also be purified, for example, through precipitation, size-exclusion chromatography, ion-exchange chromatography, filtration, ultrafiltration, microfiltration, or nanofiltration.

In certain cases, following modification and/or purification of the oligosaccharides and polysaccharides, all or part of them are then combined, as in step (f), which may be at a ratio of from 1:100 to 1:1 polysaccharide:oligosaccharide, from 1:10 to 1:1, from 1:90 to 1:2, from 1:80 to 1:3, from 1:70 to 1:4, or from 1:60 to 1:5. The specific ratio can depend on the desired properties of the final ingredient as well as the modifications and purifications that have been applied to the saccharides. In certain embodiments, it is not required to recombine all of the removed oligosaccharides and polysaccharides.

In step (f) the combining can be done in a variety of ways, for example, by mixing a solution comprising all or part of the one or more soluble polysaccharides and a solution and/or suspension comprising all or part of the one or more removed oligosaccharides, which may further be spray-dried, lyophilized, or condensed in some other way. The soluble polysaccharides and the removed oligosaccharides may also be combined by mixing a dry form comprising all or part of the one or more removed oligosaccharides produced by spray-drying, lyophilization, or condensation in some other way after removing them in step (e), with a dry form comprising all or part of the one or more soluble polysaccharides, produced by spray-drying, lyophilization, or condensation in some other way after removing them in step (c). Alternatively, one of (i) the one or more soluble polysaccharides or (ii) the removed oligosaccharides may be in dry form and the other in solution when they are combined.

When the ingredient is in a dry form, the method may further comprise a step (g) of mixing and dissolving the ingredient in a liquid to form a liquid ingredient. In various cases, the liquid may be an aqueous solution, such as water.

The ingredient formed in step (f), and the liquid ingredient formed in step (g) and of the second aspect of the disclosure, may comprise various oligosaccharides and at varying amounts depending on the desired properties. In some cases, the ingredient and liquid ingredient may comprise at least 20% by dry weight or at least 30% by dry weight cello-oligosaccharides having a degree of polymerization of from two to six, the ingredient may comprise at least 20% by dry weight or at least 30% by dry weight xylo-oligosaccharides having a degree of polymerization of from two to twelve, the ingredient may comprise at least 20% by dry weight or at least 30% by dry weight mixed-linkage glucan oligosaccharides having a degree of polymerization of from two to five, the ingredient may comprise at least 20% by dry weight or at least 30% by dry weight manno-oligosaccharides having a degree of polymerization of from two to twelve, the ingredient may comprise at least 20% by dry weight or at least 30% by dry weight xyloglucan oligosaccharides having a degree of polymerization of from four to twelve, and/or the ingredient may comprise at least 20% by dry weight or at least 30% by dry weight chito-oligosaccharides having a degree of polymerization of from two to twelve. In some instances, the ingredient can comprise a maximum of 100% by dry weight of the above oligosaccharides and the polysaccharides described herein, therefore the above embodiments, wherein the oligosaccharides are present in at least 20% by dry weight, does not comprise five or six types of oligosaccharides.

The ingredient and liquid ingredient may comprise at least 50%, 60%, 70%, 80%, 90%, 95%, 99%, or 99.5% by dry weight of saccharide present. The ingredient and liquid ingredient may consist essentially of saccharides. For example, the ingredient may have less than 0.5% by dry weight or less than 0.3% by dry weight, for instance, 0.1% by dry weight, of other substances.

In certain cases, the liquid ingredient can have a viscosity of from 5 to 100,000 cps, from 10 to 80,000 cps, from 20 to 60,000 cps, from 30 to 40,000 cps, from 40 to 20,000 cps, or from 50 to 10,000 cps. When the liquid ingredient is to be included into beverages, low syrup viscosities may be desirable, such as about 20 to 300 cps, 50 to 200 cps, or 100 to 150 cps. Higher viscosity values may be desired in applications such as chocolate making, thus where the liquid ingredient is desired to be in a syrup it may have a viscosity in the range of about 8,000 to 100,000, about 10,000 to 50,000 cps, or about 15,000 to 25,000 cps. The viscosity values are in accordance with testing using a Brookfield HDB VE roto-viscometer using standard testing procedures, wherein a 400 mL sample was taken in a tall-form beaker to ensure that no container effects occur. The instrument is operated as per the manufacturer's instructions with respect to ranges (rotoviscometry using spindle code 61, spindle speed 100 rpm, and at 22° C.).

In some instances, the liquid ingredient can have a flow rate of 100 to 350 seconds, 150 to 300 seconds, or 200 to 250 seconds. “Flow rate,” as used herein, generally refers to the volume of fluid which passes per unit time. The flow rate values specified herein, unless indicated otherwise, are determined by measuring by timing the flow rate of 5 mL of the liquid ingredient from a vertically stood syringe (BD Plastipak 300613) filled with 20 mL of test liquid under gravity at room temperature.

In various cases, the liquid ingredient may have a concentration of oligo-saccharides of from 1 to 200% w/v, 10 to 150% w/v, 20 to 140% w/v, 30 to 130% w/v, 40 to 120% w/v, 50 to 115% w/v, or 60 to 110% w/v.

In some cases, the liquid ingredient may have a concentration of polysaccharides of from 0.1 to 50% w/v, 0.2 to 40% w/v, 0.3 to 30% w/v, 0.5 to 20% w/v, or 1 to 20% w/v.

In certain cases, the liquid ingredient may have a concentration of the total of oligosaccharides and polysaccharides of from 1 to 200% w/v, 10 to 160% w/v, 20 to 150% w/v, 30 to 140% w/v, 40 to 130% w/v, 50 to 120% w/v, or 60 to 110% w/v. In various cases, the higher the concentration of the oligosaccharides and polysaccharides in the liquid, the thicker and more viscous the liquid may become. The liquid ingredient may be a homogeneous solution.

In another aspect, the ingredient and liquid ingredient may comprise at least two of the oligosaccharides. The amounts of each of the oligosaccharides may be varied depending on the desired properties of the resulting foodstuff, cosmetic, or nutraceutical. The two oligosaccharides may be present in a ratio of 1:9 to 9:1 or 1:2 to 2:1. Further, the ingredient and liquid ingredient may comprise three of the oligosaccharides, they may comprise four oligosaccharides, they may comprise five oligosaccharides, or they may comprise six oligosaccharides.

The ingredient and liquid ingredient can comprise the cello-oligosaccharides, for example, cello-oligosaccharides in combination with the xylo-oligosaccharides. Alternatively, the ingredient and liquid ingredient can comprise the cello-oligosaccharides in combination with the manno-oligosaccharides.

The one or more soluble polysaccharides in the ingredient may be particularly soluble in water or alkali. For example, soluble polysaccharides used in the disclosure can include hemicelluloses such as xylans, mannans, mixed-linkage glucans, and certain cellulose derivatives such as cellulose acetate, hydroxyethylcellulose, and hydroxymethylcellulose, and chitosan. In some embodiments, the one or more soluble polysaccharides can comprise hemicellulose. In certain embodiments, the hemicellulose can comprise xylan and/or mannan.

In some instances, the ingredient and liquid ingredient can comprise at least 2% by dry weight or at least 3% by dry weight of xylan. In various instances, the ingredient and liquid ingredient can comprise at least 2% by dry weight or at least 3% by dry weight of mannan. In certain instances, the ingredient and liquid ingredient can comprise at least 2% by dry weight or at least 3% by dry weight of cellulose derivative. In some cases, the ingredient and liquid ingredient can comprise at least 2% by dry weight or at least 3% by dry weight of mixed-linkage glucan. In various cases, the ingredient and liquid ingredient can comprise at least 2% by dry weight or at least 3% by dry weight of xyloglucan. In certain cases, the ingredient and liquid ingredient can comprise at least 2% by dry weight or at least 3% by dry weight of chitosan.

In certain cases, the ingredient and liquid ingredient can comprise of from 2 to 40% by dry weight of the one or more soluble polysaccharides, which includes polysaccharide derivatives, from 3 to 30% by dry weight of the one or more soluble polysaccharides, from 5 to 25% by dry weight of the one or more soluble polysaccharides, or from 8 to 20% by dry weight of the one or more soluble polysaccharides.

In some cases, the ingredient and liquid ingredient the ratio of polysaccharide:oligosaccharide is from 1:100 to 1:1, from 1:10 to 1:1, from 1:90 to 1:2, from 1:80 to 1:3, from 1:70 to 1:4, or from 1:60 to 1:5.

The produced ingredient and liquid ingredient may be useful in applications in which oligosaccharides, sugar, bulking sweeteners, low-intensity sweeteners, or other related food ingredients are conventionally used. For example, as sweeteners, bulking agents, added dietary fiber, or humectants. Of particular note is the use of reducing cane sugar in food products. It may be incorporated into cakes, bread, or other baked goods; chocolate or other confectionery such as toffee, fudge, meringue, jam, jelly or caramel; or drinks, for example, to provide favorable taste or color characteristics or to increase dietary fiber content. Or the ingredient may be incorporated into animal feed, for example, either as an isolated ingredient or by utilizing the enzymatic reaction mixture directly as feed.

Compositions or ingredients as described herein may be used to alter one or more properties of a finished product. Such properties include, but are not limited to, sweetness, texture, mouthfeel, binding, glazing, smoothness, moistness, viscosity, color, hygroscopicity, flavor, bulking, water-retention, caramelization, surface texture, crystallization, structural properties, reduced calories, reduced glycemic index, reduced glycemic load, increased fiber, reduced sugar, and dissolution. These may be improvements over what is currently possible with saccharides of different types, sugar substitutes, and/or other such compounds.

In the cosmetics industry, the ingredient may improve texture and moisture retention, act as UV-absorbing molecules, maintain a gel or cream structure, and/or serve as bulking agents. Furthermore, the ingredient and liquid ingredient may be useful in nutraceutical compositions, as the dietary fiber it provides has been shown to encourage digestive health, well-regulated gut flora, and other benefits to wellbeing. In this context the ingredients provided herein may also function as an ingredient in a probiotic drink or other prebiotic or probiotic formulation.

The detailed description is further supplemented with reference to the following numbered embodiments. 1) A method for producing a foodstuff, cosmetic, or a nutraceutical ingredient comprising one or more oligosaccharides and one or more soluble polysaccharides comprising the steps of: (a) providing a plant biomass comprising one or more soluble polysaccharides and one or more insoluble polysaccharides; (b) treating the plant biomass to dissolve the one or more soluble polysaccharides; (c) removing a portion of the dissolved one or more soluble polysaccharides; (d) reacting the remaining plant biomass with one or more enzymes to form one or more oligosaccharides; (e) removing the one or more oligosaccharides; and (f) combining the portion of the dissolved one or more soluble polysaccharides from step (c) and the one or more oligosaccharides from step (e) to form the ingredient. 2) The method according to numbered embodiment 1, wherein the treating in step (b) is thermochemical treatment. 3) The method according to numbered embodiment 2, wherein the thermochemical treatment is hot water treatment or hot alkali treatment. 4) The method according to numbered embodiment 3, wherein the alkali treatment uses an alkali with a pH of from 10 to 14. 5) The method according to either numbered embodiment 3 or 4, wherein the alkali treatment uses sodium hydroxide, potassium hydroxide, sodium carbonate, calcium carbonate, calcium hydroxide, ammonium sulfate, ammonium hydroxide, and aqueous ammonia. 6) The method according to any preceding numbered embodiment, wherein the treating in step (b) occurs at a temperature of from 30 to 180° C. 7) The method according to any preceding numbered embodiment, wherein the treating in step (b) occurs for 10 minutes to 24 hours. 8) The method of any preceding numbered embodiment, wherein after the removing of the one or more oligosaccharides, the one or more oligosaccharides and/or dissolved one or more soluble polysaccharides undergo chemical, physical, or enzymatic treatment, such as reduction, oxidation, caramelization, or Maillard reaction. 9) The method of any preceding numbered embodiment, wherein the dissolved one or more soluble polysaccharides and/or the one or more oligosaccharides are dried before being combined together in step (f). 10) The method according to numbered embodiment 9, wherein the method further comprises a step: (g) mixing and dissolving the ingredient in a liquid to form a liquid ingredient, wherein the liquid ingredient has a viscosity of from 5 to 100,000 cps. 11) The method according to numbered embodiment 10, wherein the concentration of the oligosaccharides and polysaccharides in the liquid ingredient is of from 1 to 200% w/v. 12) The method according to any preceding numbered embodiment, wherein the one or more soluble polysaccharides comprise at least one selected from the group consisting of: mannans, mixed-linkage glucans, lignocellulose, hemicellulose, certain cellulose derivatives such as cellulose acetate, hydroxyethylcellulose and hydroxymethylcellulose, and chitosan. 13) The method according to numbered embodiment 12, wherein the hemicellulose comprises a xylan and/or a mannan. 14) The method according to any preceding numbered embodiment, wherein the plant biomass comprises a sugar cane biomass, a corn biomass, a wheat biomass, a hardwood, or a softwood. 15) A foodstuff, cosmetic, or a nutraceutical ingredient obtainable by the method of any preceding numbered embodiment. 16) A foodstuff, cosmetic, or nutraceutical liquid ingredient comprising at least one oligosaccharide selected from the list consisting of: i) cello-oligosaccharide having a degree of polymerization of from two to six; ii) xylo-oligosaccharide having a degree of polymerization of from two to twelve; iii) manno-oligosaccharide having a degree of polymerization of from two to twelve; iv) mixed-linkage glucan oligosaccharide having a degree of polymerization of from two to five; v) xyloglucan oligosaccharide having a degree of polymerization of from four to twelve; and vi) chito-oligosaccharide having a degree of polymerization of from two to twelve; and at least one polysaccharide selected from the list consisting of: i) xylan; ii) mannan; iii) cellulose derivative; iv) mixed-linkage glucan; v) xyloglucan; and vi) chitosan; wherein the liquid ingredient comprises at least 20% by dry weight of the at least one oligosaccharide and at least 2% by dry weight of the at least one polysaccharide, and wherein the liquid ingredient has a viscosity of from 5 to 100,000 cps, 8,000 to 100,000 cps, 10,000 to 50,000 cps, or 15,000 to 25,000 cps. 17) The liquid ingredient of numbered embodiment 16, wherein the liquid ingredient comprises at least two of the oligosaccharides listed in (i) to (vi). 18) The liquid ingredient of either numbered embodiment 16 or numbered embodiment 17, wherein the liquid ingredient comprises at least 20% by dry weight of the cello-oligosaccharides having a degree of polymerization of from two to six. 19) The liquid ingredient of any one of numbered embodiments 16 to 18, wherein the liquid ingredient comprises at least 20% by dry weight of the xylo-oligosaccharide having a degree of polymerization of from two to twelve. 20) The liquid ingredient of any one of numbered embodiments 16 to 19, wherein the liquid ingredient comprises at least 20% by dry weight of the manno-oligosaccharide having a degree of polymerization of from two to twelve. 21) The liquid ingredient of any one of numbered embodiments 16 to 20, wherein the liquid ingredient comprises at least 2% by dry weight of the xylan. 22) The liquid ingredient of any one of numbered embodiments 16 to 21, wherein the liquid ingredient comprises at least 2% by dry weight of the mannan. 23) The liquid ingredient of any one of numbered embodiments 16 to 22, wherein the liquid ingredient comprises at least 2% by dry weight of the cellulose derivative. 24) The liquid ingredient of any one of numbered embodiments 16 to 23, wherein the liquid ingredient has a concentration of polysaccharides of from 0.1 to 50% (w/v). 25) The liquid ingredient of any one of numbered embodiments 16 to 24, wherein the liquid ingredient has a concentration of oligosaccharides of from 1 to 200% (w/v). 26) The liquid ingredient of any one of numbered embodiments 16 to 25, wherein the liquid ingredient has a concentration of polysaccharides and oligosaccharides of from 1 to 200% (w/v). 27) The liquid ingredient of any one of numbered embodiments 16 to 26, wherein the liquid ingredient comprises an amount of polysaccharide and oligosaccharide in a ratio from 1:100 to 1:1. 28) The liquid ingredient of any one of numbered embodiments 16 to 27, wherein the liquid ingredient comprises two oligosaccharides in a ratio from 1:9 to 9:1 in relation to each other. 29) Use of the liquid ingredient of any of numbered embodiments 16 to 28 in a foodstuff, cosmetic, or nutraceutical product.

Additional Exemplary Embodiments of Pretreating Biomass to Remove Monosaccharides and/or Disaccharides

In some cases, the present disclosure relates to novel methods of physically and thermochemically treating plant biomass materials for the production of foodstuff, cosmetic, or nutraceutical ingredients.

Sugary foods and drinks are an important part of culture and lifestyle habits across the world, but the sugar they contain has been linked to obesity, diabetes, poor dental health, and disruptive behavior in people. Because of this, consumer preferences have been shifting away from sugar-containing foods, and governments are increasingly implementing regulation to encourage the consumption of less sugar.

As such, industry has been searching for suitable low-calorie sweeteners for many decades to substitute for sugar in food and beverages. Unfortunately, many sugar substitutes are produced from non-natural resources, and often offer bitter undertones or other unpleasant tastes along with their sweetness, both of which consumers find unappealing. Moreover, while many sweeteners are able to mimic the sweetness of sugar in food and drinks, few are able to mimic the broad range of roles that sugar plays in food, such as adding bulk, modulating texture, providing structure, acting as a preservative, and modulating color and flavor through caramelization and Maillard reactions.

Dietary fiber is an important part of a positive diet and helps maintain digestive health and a well-regulated gut flora. Such fiber comprises saccharides of varying chain lengths and types. In addition to being found naturally in a wide spectrum of foods, fiber can also be produced separately and added to other foods during their manufacture.

Biomass is a good source of saccharides that can be used to replace sugar and add fiber to food products. Compositions made from feedstocks have been provided. However, there remains a need to optimize the process by which these saccharides are obtained from the biomass and processed into the compositions useful as a foodstuff, cosmetic, or nutraceutical ingredient on a large and commercial scale. Enzyme breakdown of a large amount of plant biomass can take a considerable amount of time. Furthermore, the saccharides generally need to be produced by controlled breakdown. Enzyme breakdown can be desirable for this as product sizes can be exquisitely controlled, ensuring no and/or little monosaccharides are yielded.

Methods, as provided herein, can economically and efficiently enable the production of a foodstuff, cosmetic, or nutraceutical ingredient from a plant biomass starting material which is quicker than previously used methods, yields a purer final product, and can be used on a large and commercial scale. The method can do so by performing a prewashing step to remove from biomass endogenous monosaccharides and/or disaccharides, and employing thermochemical pretreatment steps that may ensure controlled breakdown and release of the saccharides that can be needed to manufacture the ingredient. Together, these steps may ensure that no and/or little monosaccharides are yielded during pre-enzyme processing. This can maximize efficiency and limit the amount of post-reaction purification needed.

Accordingly, in another aspect of the disclosure there is provided a method for producing a foodstuff, cosmetic, or nutraceutical ingredient, the ingredient comprising one or more oligosaccharides, wherein the method comprises the steps of:
a) a physical pretreatment of a plant biomass comprising monosaccharides and/or disaccharides;
b) a washing cycle (also referred to herein as an incubation cycle) comprising the steps of (i) washing (e.g., incubating) the plant biomass to solubilize at least a portion of the monosaccharides and/or disaccharides and (ii) removing the at least a portion of monosaccharides and/or disaccharides;
c) a thermochemical pretreatment of the plant biomass;
d) forming the one or more oligosaccharides by an enzymatic reaction, the enzymatic reaction comprising the step of contacting, in a solution or suspension, one or more polysaccharide-cleaving enzymes and the plant biomass;
e) separating (also referred to herein as enriching for or isolating) the one or more oligosaccharides from the enzymatic reaction mixture and using the one or more oligosaccharides to form the ingredient.

Preparing the foodstuff, cosmetic, or nutraceutical ingredient in the manner provided herein can allow for: efficient use of biomass by incorporating oligomeric and polymeric material from the same biomass source, purification, derivatization or other modification, as well as control of oligomeric and polymeric proportions, which can improve the functional properties, nutritional properties, and tolerance of the ingredient.

Steps (a), (b), and (c) of the method of the disclosure are all “pretreatment” steps which are performed on the plant biomass starting material. “Pretreatment,” as used herein, generally refers to steps that are performed on the plant biomass before a polysaccharide-cleaving enzyme is put into contact with the plant biomass.

Step (a) is a physical pretreatment of the plant biomass, which can have the purpose of physically breaking down the plant biomass in preparation for the subsequent steps. The physical step may help speed up the overall method because it may increase the available surface area of the plant biomass enabling the chemicals used in subsequent steps to be active on more of the plant biomass, for example, at one time. The physical pretreatment step may comprise chipping, chopping, milling, ball-milling, grinding, sprucing, blending, or a combination thereof, of the plant biomass.

Any substance which comprises suitable polysaccharides may be the plant biomass. As the foodstuff, cosmetic, and nutraceutical industries use a broad variety of oligosaccharides, the polysaccharides suitable in the method are not particularly limited. Plant biomass suitable for producing the oligosaccharide profile of the current disclosure may comprise, for example, cellulose, lignocellulose, chitin, chitosan, xylan (such as glucuronoxylan, arabinoxylan, and glucuronoarabinoxylan) xyloglucan, and mixed-linkage glucan, and/or mannan (such as glucomannan, galactomannan, or galactoglucomannan). However, any plant biomass which can be suitably acted upon is envisaged.

As such, the plant biomass may be grain, grain chaff, bean pods, seed coats, and/or other seed materials; seaweeds; corn stover, straw, bagasse, miscanthus, sorghum residue, switch grass, bamboo, and/or other monocotyledonous tissue; water hyacinth, leaf tissue, roots, and/or other vegetative matter; and/or any combination of suitable plant biomasses. In some embodiments, the plant biomass can comprise sugar cane, corn stover, corncob, wheat bran, wheat straw, hardwood or softwood. In certain embodiments, the plant biomass can comprise corncob.

Step (b) is a washing cycle (or incubation cycle) pretreatment of the plant biomass that can occur after the physical pretreatment step. The aim of step (b) may be to solubilize and remove monosaccharides and/or disaccharides from the biomass. For example, the monosaccharides and/or disaccharides may include, but are not limited to, free sucrose, maltose, lactose, glucose, fructose, or galactose. Removal of free disaccharides such as sucrose can be of interest, as disaccharides cannot subsequently be easily removed from the oligosaccharide fraction, for example, filtration methods can be used but would generally cause equal loss of other disaccharides.

Step (i) of the washing cycle may occur at a range of temperatures, for example, of from 5 to 150° C., from 10 to 100° C., or from 15 to 50° C. In some cases, the washing cycle can occur at room temperature, for instance, at about 15 to 25° C. or about 20 to 22° C. Higher temperatures may allow for quicker solubilization of the monosaccharides and/or disaccharides, however, too high temperatures can be more difficult to achieve in an efficient and cost-effective manner and may damage the biomass compounds or solubilize compounds that are not desirable to be solubilized during this step.

Step (i) of the washing cycle may occur for a range of time scales, for example, large amounts of biomass may be exposed to this step for a longer period of time, which can be adjusted accordingly. For example, the time scale may be of from 0.5 minutes to 72 hours, from 1 minute to 12 hours, from 5 minutes to 24 hours, or from 10 minutes to 3 hours. In certain embodiments, this step may occur as batch or continuous.

In some embodiments, step (i) of the washing cycle may comprise washing the plant biomass at room temperature in water, i.e., water supplied at a neutral pH of about pH 7. In another aspect, the step (i) of the washing cycle may comprise heating the plant biomass in water, i.e., water supplied at a neutral pH of about pH 7. During the washing cycle, the neutral water supplied may become slightly acidic as monosaccharides and/or disaccharides are solubilized.

In certain embodiments, step (i) of the washing cycle may comprise heating the plant biomass in an alkali solution having a pH of from 7.1 to 14, from 7.5 to 12, or from 8 to 11. The solution may comprise, or suitably consist of, any one of the alkalis selected from: sodium hydroxide, potassium hydroxide, sodium carbonate, calcium carbonate, calcium hydroxide, ammonium sulfate, ammonium hydroxide, and aqueous ammonia. In various embodiments, the alkali may be sodium hydroxide. A combination of the listed alkalis is also envisaged.

In various cases, step (i) of the washing cycle may comprise heating the plant biomass in an acidic solution having a pH of from 1 to 6.9, from 2 to 6.5, or from 4 to 6. The solution may comprise, or suitably consist of, any organic or mineral acids, such as one of the acids selected from: sulfuric acid, hydrochloric acid, nitric acid, phosphoric acid, acetic acid, maleic acid, fumaric acid, and oxalic acid. In certain cases, the acid may be sulfuric acid. A combination of the listed acids is also envisaged.

In certain instances, a portion or all of the monosaccharides and/or disaccharides in the plant biomass are solubilized and removed during step (b). Other contaminants may also be removed during this step, such as other soluble sugars and minerals.

Step (b) may be repeated to remove monosaccharides and/or disaccharides that were not removed from the plant biomass during the washing cycle. In various instances, step (b) may be performed at least two times, at least three times, at least four times, or at least five times. Step (c) is a thermochemical pretreatment of the plant biomass that can occur after the physical and washing pretreatment steps. “Thermochemical,” as used herein, generally refers to heating above room temperature (room temperature is, for instance, about 15 to 25° C. or about 20 to 22° C.) the plant biomass in a chemical, such as heating in a solution of water, acid, or alkali. The purpose of the thermochemical step can be to help speed up the overall method because it may chemically modify chemical components of the plant biomass, for example, it can disrupt hydrogen bonds between saccharides enabling the enzyme in the subsequent step to more easily break up the saccharides.

The heating may be at a range of temperatures, for example, from 50 to 150° C., from 60 to 130° C., from 65 to 120° C., or from 70 to 110° C. Higher temperatures can allow for quicker chemical and/or physical modification, however, too high temperatures can be more difficult to achieve in an efficient and cost-effective manner.

The heating may occur for a range of time scales, particularly large amounts of biomass may be exposed to heating for a longer period of time, which can be adjusted accordingly. For example, the heating of the plant biomass can be of from 5 minutes to 72 hours, from 15 minutes to 24 hours, from 30 minutes to 12 hours, or from 1 hour to 4 hours.

In some embodiments, the thermochemical pretreatment may comprise heating the plant biomass in water, i.e., at a neutral pH of about pH 7.

In certain embodiments, the thermochemical treatment may comprise heating the plant biomass in an alkali solution having a pH of from 7.1 to 14, from 9 to 13, or from 10 to 13. The solution may comprise, or suitably consist of, any one of the alkalis selected from: sodium hydroxide, potassium hydroxide, sodium carbonate, calcium carbonate, calcium hydroxide, ammonium sulfate, ammonium hydroxide and aqueous ammonia. In various embodiments, the alkali may be sodium hydroxide. A combination of the listed alkalis is also envisaged.

In some cases, the thermochemical treatment may comprise heating the plant biomass in an acidic solution having a pH of from 1 to 6.9, from 2 to 6.5, or from 4 to 6. The solution may comprise, or suitably consist of, any organic or mineral acids, such as one of the acids selected from: sulfuric acid, hydrochloric acid, nitric acid, phosphoric acid, acetic acid, maleic acid, fumaric acid, and oxalic acid. In certain cases, the acid may be sulfuric acid. A combination of the listed acids is also envisaged.

Multiple different sequential thermochemical treatment steps are also envisaged for step (c). For example, step (c) may be performed at least two times, at least three times, at least four times, or at least five times.

In certain cases, the washing of step (b) may occur in water and the pretreatment of step (c) may occur in alkali.

After the pretreatment steps, step (d) may comprise the enzymatic reaction forming the one or more oligosaccharides from the plant biomass. The polysaccharides present in the plant biomass may be partially cleaved by enzymes into oligosaccharides (e.g., useful oligosaccharides), leaving partially cleaved, or uncleaved, polysaccharides, which may include cellulose, xylan (such as glucuronoxylan, arabinoxylan, or glucuronoarabinoxylan), mannan (such as glucomannan, galactomannan, or galactoglucomannan), mixed-linkage glucan, xyloglucan chitin, chitosan, or lignocellulose.

The reaction may take place in solution and/or suspension. The reaction may take place in a suitable reaction vessel. In some cases, the reaction may take place at a temperature or temperature protocol suitable for the particular combination of enzyme and plant biomass, the reaction may be allowed to progress for a certain amount of time, until the products have reached a desired concentration, or until some other requirement has been met and the products are isolated or otherwise collected. This time period may be from about 1 minute to about 6 days, from about 0.5 days to about 5 days, or from about 16 hours to about 96 hours. The reaction may alternatively be allowed to proceed until no further catalysis occurs.

In order to ensure optimal contact between the enzymes and the plant biomass, the reaction mixture may be agitated, either constantly or at intervals. The agitation may take the form of rhythmically moving the entire reaction vessel, of a fan or other stirring device, of a bubble sparging, or any other method of agitation.

The enzymatic reaction may be a microbial fermentation. The temperature and reaction time may be suitable for the growth of the microbial organism used. The microbial organism may be genetically altered to produce an enzyme suitable for the production of an oligosaccharide of the present disclosure. The microbe may be, for example, a bacterium, for example, Escherichia coli, or a fungus, such as Saccharomyces cerevisiae or Trichoderma reesei.

Further embodied in the present disclosure is an expression vector suitable for modifying the subject microorganism such that it produces an enzyme or mixture of enzymes of the current disclosure. Where desired, the expression vector, which may be a plasmid or any other nucleic acid able to induce production of the enzyme, may comprise one or more of the following regulatory sequences so as to control the expression of the exogenous enzyme: regulatory sequences of a heat shock gene, regulatory sequences of a toxicity gene, and regulatory sequences of a spore formation gene.

The enzymatic reaction can be carried out at a temperature or temperature protocol suitable to the enzymes and substrates used. For example, it may be carried out at a constant temperature in the range of from about 10° C. to about 100° C., about 20° C. to about 70° C., or about 30° C. to about 40° C. If the enzymatic reaction takes the form of a microbial fermentation, the temperature may be suitable for such, for example, the enzymatic reaction may comprise the growth of E. coli and/or the temperature may be constant and about 37° C.

The pH of the solution or suspension may affect the activity of the enzymes. Control of pH may assure that an enzymatic reaction proceeds at a suitable rate. The enzymatic reaction of the present disclosure may take place at a pH in the range of from about 2 to about 10, about 3 to about 8, or about 4 to about 6.

The enzymatic reaction may be allowed to continue to run until there is 5-75% undigested polysaccharide-containing plant biomasses remaining, 5-70%, 5-65%, 5-55%, more or 10-50%. This can be monitored or checked by reducing end assays, such as the anthrone assay and/or by chromatographic methods such as thin-layer chromatography and high-performance anion exchange chromatography.

There are many enzymes that may be suitable for use in the enzymatic reaction of the present method. For example, “lytic polysaccharide monooxygenase” and “LPMO” which are a class of enzymes able to oxidatively cleave polysaccharides using a copper comprising moiety and using an oxygen source, such as a molecule of dioxygen, peroxide, or any other oxygen source; and a suitable reducing agent. As such, when an LPMO is used, the enzymatic reaction may be carried out under aerobic conditions. Suitable reducing agents are not particularly limited, but examples include ascorbic acid, gallic acid, cysteine, NADH, NADPH, pyrogallol, dithiothreitol, cyanoborohydrides, borohydrides, photosynthetic pigments, lignin, lignols, and a combination of cellobiose and cellobiose dehydrogenase. A wide variety of photosynthetic pigments may be used, for example, thylakoids and purified fractions or chlorophyllin and light may be supplied. LPMOs can be selected from the following families: AA9, AA10, AA11, AA13, AA14 and AA15. The LPMO may be PaLPMO9E (SEQ ID NO:1), an AA9 LPMO originally isolated from the ascomycete fungus Podospora anserina. The LPMO may be an AA9 LPMO from Trichoderma reesei (SEQ ID NO:23).

Aerobic conditions may comprise the addition of oxygen, which may be provided by aeration of the substrate mixture with an oxygen-comprising gas, such as air. Aeration may be conducted by the introduction of oxygen-comprising air bubbles into the aqueous substrate mixtures by various systems, such as an air-injector, an aeration frit, a membrane system, or an internal-loop airlift reactor. The concentration of molecular oxygen in the enzymatic reaction may be from about 4 mg/L to about 14 mg/L.

Another type of enzyme that can be used in the method is a “cellulase” which has hydrolytic activity against cellulose, for example, endo-1,4-beta-glucanase, cellobiohydrolase, and/or beta-glucosidase activities. Such enzymes are able to cleave glycosidic bonds in one or more forms of cellulose, including cellulose found in plant biomass. In doing so they produce products including glucose and cello-oligosaccharides. Beta-glucanases include enzymes from GH5, GH7, and GH12 enzyme, such as those derived from Aspergillus niger (SEQ ID NOs: 12, 13, and 14) and Trichoderma reesei (SEQ ID NOs: 24 and 25).

Another type of enzyme is “cellobiohydrolase” that has hydrolytic activity against cellulose, and produces mainly cellobiose as a product. Cellobiose is a disaccharide, and is a cello-oligosaccharide. Such enzymes are able to cleave glycosidic bonds in one or more forms of cellulose, including cellulose found in plant biomass. Cellobiohydrolases may be from GH6 and GH7 enzyme families or Cel6A or Cel7A enzymes derived from Trichoderma reesei (SEQ ID NOs: 10 and 11).

Another type of enzyme is “beta-glucosidase” that has hydrolytic activity against cellulose, and produces mainly glucose as a product. Such enzymes are able to cleave glycosidic bonds in one or more forms of cellulose, including cellulose found in plant biomass. Beta-glucosidases may include GH3 beta-glucosidases, such as from Trichoderma reesei (SEQ ID NO: 22).

Another type of enzyme is a lichenase, which may be selected from: the GH5, GH7, GH8, GH9, GH12, GH16, GH17, or GH26 families. In some cases, the lichenase may be a GH16 enzyme. The GH16 enzyme may be derived from Bacillus subtilis (SEQ ID NO:2). The enzyme is able to act on, for example, mixed-linkage glucans, which are glucans comprising a mixture of β-1,3 and β-1,4 linkages, and may cleave them at β-1,4 glycosidic bonds. In the case in which the lichenase acts on a mixed-linkage glucan, the β-glucans produced may fall largely within the size range of from about 3 to about 7 residues, so they may be useful in the food, cosmetics, and nutraceutical industries. Mixed-linkage glucans are abundant in members of the grass and horsetail families, and as such, grass-based biomasses such as straw have high levels of it, and may be acted upon usefully with lichenases.

Another type of enzyme is a xylanase, which may act on, for example, plant biomass comprising a xylan backbone. The xylanase may be, for example, a glucuronoxylanase, an arabinoxylanase, or a glucuronoarabinoxylanase. The enzyme may be active on a variety of polymers having a xylan backbone, such as glucuronoxylan, arabinoxylan, and glucuronoarabinoxylan. These polymers are abundant in various plant biomass, for example, both hardwood and softwood may comprise suitable polysaccharides, with hardwood often comprising glucuronoxylan and softwood often comprising arabinoglucuronoxylan. In some embodiments, xylanases may include GH5 xylanases from Ruminiclostridium thermocellum (SEQ ID NO:3) and Gonapodya prolifera (SEQ ID NO:4), and GH30 xylanases from Dickeya chrysanthemi (SEQ ID NO: 5), Bacillus subtilis (SEQ ID NO:6), Bacteroides ovatus (SEQ ID NO:7), and Trichoderma reesei (SEQ ID NO:15).

Other enzymes useful in the disclosure may include xyloglucanases and xyloglucan endoglucanases (XEGs), which are produced by numerous organisms, including plant-pathogenic microbes. They are able to act on xyloglucan, a hemicellulosic β-1,4 glucan chain abundant in the primary cell wall of higher plants, which is decorated with xylose, some of the xylose residues being further decorated with other residues, such as galactose. When suitable xyloglucanases or XEGs act on xyloglucan, the products comprise xyloglucan oligosaccharides having a main chain of a length that can be useful in the foodstuff, cosmetics, and nutraceutical industries. Xyloglucanases can include a GH5 xyloglucanase from Bacteroides ovatus (SEQ ID NO:8) and a GH74 xyloglucanase from Trichoderma reesei.

As any given natural plant biomass is likely to comprise a mixture of different polysaccharides, it can sometimes be the case that a mixture of different enzymes is beneficial. Such a mixture may comprise one or more of any other enzyme. For example, such a mixture might comprise an LPMO with an endo-glucanase, a xylanase with a lichenase, a cellobiohydrolase with a mannanase, or an endo-glucanase with a cellobiohydrolase in which the enzyme partners are present in molar ratios from 1:100 and 100:1.

In some instances, the one or more enzymes may be a cocktail of different enzymes, for example, a crude or semi-crude enzyme preparation. The term “crude enzyme preparation,” as used herein, generally refers to a soluble preparation extracted from a microbial fermentation that has undergone minimal processing after the extraction. For example, typically the preparation may only undergo filtration in order to remove insoluble components. The term “semi-crude enzyme preparation,” as used herein, generally refers to a soluble preparation extracted from a microbial fermentation that has undergone some processing after the extraction, for example, the preparation may undergo filtration in order to remove insoluble components, increasing the enzyme concentration and/or nanofiltration to remove small molecular weight compounds.

In some cases, the crude or semi-crude enzyme preparation may be from a bacteria or a fungus. For example, the preparation may be from a fungus, such as a filamentous cellulolytic fungus, such as from Trichoderma or Aspergillus species. The enzyme may be a crude or semi-crude enzyme preparation from a Trichoderma reesei strain.

In step (e), the oligosaccharides may be separated from the enzymatic reaction mixture in a number of ways. They may be isolated based on solubility, so that a composition of soluble saccharides only is extracted for further processing, and/or isolated chromatographically to produce a composition with a narrower band of oligosaccharide chain lengths. Isolation may, for example, be based on precipitation, size-exclusion chromatography, ion-exchange chromatography, filtration, ultrafiltration, microfiltration, or nanofiltration. In the case that isolation based on solubility is carried out, the profile of saccharides present in the isolated composition may depend on the original enzymatic reaction, as different saccharides decrease in solubility with length at different rates.

Also envisaged in the scope of the present disclosure is the further treatment of all or part of the produced oligosaccharides to produce further products before incorporation into a foodstuff, cosmetic, or nutraceutical. This further treatment may comprise any chemical, physical, or enzymatic step, such as reduction, for example, reductive amination where suitable; oxidation, caramelization, modification with a Schiff base, or via the Maillard reaction, or by any combination of such steps, and may provide different products having properties which are improved for the desired purpose. For example, the caramelization properties, calorific value, flavor, and color may be modified. The oligosaccharides may also be purified, for example, through precipitation, size-exclusion chromatography, ion-exchange chromatography, filtration, ultrafiltration, microfiltration, or nanofiltration.

The ingredient formed in step (e) may comprise various oligosaccharides and at varying amounts depending on the desired properties. In various cases, the ingredient may comprise at least 20% by dry weight or at least 30% by dry weight cello-oligosaccharides having a degree of polymerization of from two to six, the ingredient may comprise at least 20% by dry weight or at least 30% by dry weight xylo-oligosaccharides having a degree of polymerization of from two to twelve, the ingredient may comprise at least 20% by dry weight or at least 30% by dry weight mixed-linkage glucan oligosaccharides having a degree of polymerization of from two to five, the ingredient may comprise at least 20% by dry weight or at least 30% by dry weight manno-oligosaccharides having a degree of polymerization of from two to twelve, the ingredient may comprise at least 20% by dry weight or at least 30% by dry weight xyloglucan oligosaccharides having a degree of polymerization of from four to twelve, and/or the ingredient may comprise at least 20% by dry weight or at least 30% by dry weight chito-oligosaccharides having a degree of polymerization of from two to twelve. In some embodiments, the ingredient can comprise a maximum of 100% by dry weight of the above oligosaccharides and the polysaccharides described herein, therefore the above embodiment, wherein the oligosaccharides are present in at least 20% by dry weight, does not comprise all six types of oligosaccharides.

In some instances, the ingredient may comprise at least 50%, 60%, 70%, 80%, 90%, 95%, 99%, or 99.5% by dry weight of saccharide present. The ingredient may consist essentially of saccharides. For example, the ingredient may have less than 0.5% by dry weight or less than 0.3% by dry weight, for instance, 0.1% by dry weight, of other substances.

In various instances, the ingredient may comprise at least two of the oligosaccharides. The amounts of each of the oligosaccharides may be varied depending on the desired properties of the resulting foodstuff, cosmetic, or nutraceutical. The two oligosaccharides may be present in a ratio of 1:9 to 9:1 or 1:2 to 2:1. Further, the ingredient may comprise three of the oligosaccharides, four of oligosaccharides, five of the oligosaccharides, or six of the oligosaccharides.

In some embodiments, the ingredient may comprise the cello-oligosaccharides, for instance, cello-oligosaccharides in combination with the xylo-oligosaccharides. In certain embodiments, the ingredient may comprise the cello-oligosaccharides in combination with the manno-oligosaccharides.

The produced ingredient can be useful in applications in which oligosaccharides, sugar, bulking sweeteners, low-intensity sweeteners, or other related food ingredients are conventionally used. For example, as sweeteners, bulking agents, added dietary fiber, or humectants. Of particular note may be in the use of reducing cane sugar in food products. The ingredient may be incorporated into cakes, bread, or other baked goods; into chocolate or other confectionery such as toffee, fudge, meringue, jam, jelly or caramel; or drinks, for example, to provide favorable taste or color characteristics or to increase dietary fiber content. In some instances, the ingredient may be incorporated into animal feed, for example, either as an isolated ingredient or by utilizing the Compositions or ingredients as described herein may be used to alter one or more properties of a finished product. Such properties include, but are not limited to, sweetness, texture, mouthfeel, binding, glazing, smoothness, moistness, viscosity, color, hygroscopicity, flavor, bulking, water-retention, caramelization, surface texture, crystallization, structural properties, reduced calories, reduced glycemic index, reduced glycemic load, increased fiber, reduced sugar, and dissolution. These may be improvements over what is currently possible with saccharides of different types, sugar substitutes, and/or other such compounds.

In the cosmetics industry, the ingredient may improve texture and moisture retention, act as UV-absorbing molecules, maintain a gel or cream structure, and/or serve as bulking agents. Furthermore, the ingredient can be useful in nutraceutical compositions, as the dietary fiber it provides has been shown to encourage digestive health, well-regulated gut flora, and other benefits to wellbeing. In this context the ingredients herein may also function as an ingredient in a probiotic drink or other prebiotic or probiotic formulation.

The detailed description is further supplemented with reference to the following numbered embodiments. 1) A method for producing a foodstuff, cosmetic, or nutraceutical ingredient, the ingredient comprising one or more oligosaccharides, wherein the method comprises the steps of: a) a physical pretreatment of a plant biomass comprising monosaccharides and/or disaccharides; b) a washing cycle comprising the steps of (i) washing the plant biomass to solubilize at least a portion of the monosaccharides and/or disaccharides and (ii) removing the at least a portion of monosaccharides and/or disaccharides; c) a thermochemical pretreatment of the plant biomass; d) forming the one or more oligosaccharides by an enzymatic reaction, the enzymatic reaction comprising the step of contacting, in a solution or suspension, one or more polysaccharide-cleaving enzymes and the plant biomass; e) separating the one or more oligosaccharides from the enzymatic reaction mixture and using the one or more oligosaccharides to form the ingredient. 2) The method of numbered embodiment 1, wherein the physical pretreatment step comprises chipping, chopping, milling, ball-milling, grinding, sprucing or blending of the plant biomass. 3) The method of either numbered embodiment 1 or numbered embodiment 2, wherein step (i) of the washing cycle occurs in water, acid, or alkali. 4) The method of any preceding numbered embodiment, wherein step (i) of the washing cycle occurs at a temperature of from 5 to 150° C., from 10 to 100° C., or from 15 to 50° C. 5) The method of any preceding numbered embodiment, wherein step (i) of the washing cycle occurs for a time scale of from 0.5 minutes to 72 hours, of from 1 minute to 12 hours, of from 5 minutes to 24 hours, or from 10 minutes to 3 hours. 6) The method of any preceding numbered embodiment, wherein the thermochemical pretreatment comprises heating the plant biomass in a solution of water, acid, or alkali. 7) The method of numbered embodiment 6, wherein the heating of the plant biomass is at a temperature of from 50 to 150° C., of from 60 to 130° C., of from 65 to 120° C., or of from 70 to 110° C. 8) The method of either numbered embodiment 6 or 7, wherein the heating of the plant biomass is of from 5 minutes to 72 hours, from 15 minutes to 24 hours, from 30 minutes to 12 hours, or from 1 hour to 4 hours. 9) The method of any one of numbered embodiments 6 to 8, wherein the solution has a pH of from 7.1 to 14, 7.5 to 12, or of from 8 to 11. 10) The method of numbered embodiment 9, wherein the solution comprises sodium hydroxide potassium hydroxide, sodium carbonate, calcium carbonate, aqueous ammonia, ammonium sulfate, or ammonium hydroxide. 11) The method of any one of numbered embodiments 6 to 8, wherein the solution has a pH of from 1 to 6.9, of from 2 to 6.5, or of from 4 to 6. 12) The method of numbered embodiment 11, wherein the solution comprises sulfuric acid, hydrochloric acid, nitric acid, phosphoric acid, acetic acid, maleic acid, fumaric acid, or oxalic acid. 13) The method of any one of numbered embodiments 1 to 12, wherein the plant biomass is sugar cane, corn stover, corncob, wheat bran, wheat straw, hardwood, or softwood. 14) The method of any one of numbered embodiments 1 to 13, wherein the plant biomass comprises cellulose, chitin, chitosan, xylan, xyloglucan, mixed-linkage glucan, mannan, or lignocellulose. 15) The method of any one of numbered embodiments 1 to 14, wherein the one or more of the polysaccharide-cleaving enzymes is one of cellulase, xylanase, xyloglucanase, endo-glucanase, cellobiohydrolase, mannanase, lichenase or a lytic polysaccharide monooxygenase (LPMO), for example, selected from the group consisting of: AA9, AA10, AA11, AA13, AA14, and AA15. 16) The method of any one of numbered embodiments 1 to 15, wherein the one or more of the polysaccharide-cleaving enzymes is prepared from T. reesei fungi. 17) The method of any one of numbered embodiments 1 to 16, wherein the one or more oligosaccharides comprise one of β-glucans, cello-, MLG-, mannan-, or xylo-oligosaccharide. 18) The method of any one of numbered embodiments 1 to 17, wherein the polysaccharide-cleaving enzyme(s) is operably linked to a catalytic or non-catalytic module, for example, wherein the polysaccharide-cleaving enzyme is operably linked to a non-catalytic module and the non-catalytic module is a carbohydrate-binding module. 19) The method of any one of numbered embodiments 1 to 18, wherein after the separating of the one or more oligosaccharides, the one or more oligosaccharides undergo chemical, physical, or enzymatic treatment, such as reduction, oxidation, caramelization, or Maillard reaction.

The detailed description is further supplemented with reference to the following numbered embodiments. 1) A method for producing an ingredient for human consumption, the method comprising: (a) physically treating a plant biomass; (b) subjecting the physically treated plant biomass to an incubation cycle comprising: (i) incubating the physically treated plant biomass in an incubation solution having a pH from 6.6 to 7.4 to solubilize monosaccharides and/or disaccharides from the physically treated plant biomass; and (ii) removing a portion of the solubilized monosaccharides and/or disaccharides from the incubation solution; (c) thermochemically treating the incubated plant biomass in one of (i) an acidic solution having a pH from 2 to 6.5 or (ii) an alkali solution having a pH from 7.5 to 12; (d) contacting, in a solution or suspension, one or more polysaccharide-cleaving enzymes and the thermochemically treated plant biomass to form one or more oligosaccharides; and (e) enriching the solution or suspension to increase the concentration of the one or more oligosaccharides to form the ingredient. 2) The method of numbered embodiment 1, further comprising removing at least a portion of the monosaccharides and/or disaccharides from the incubation solution at step (b) (ii). 3) The method any preceding numbered embodiment, wherein the thermochemically treated plant biomass comprises no or substantially no monosaccharides. 4) The method of any preceding numbered embodiment, further comprising purifying the one or more oligosaccharides from the solution or suspension. 5) The method of any preceding numbered embodiment, further comprising repeating step (b). 6) The method of numbered embodiment 5, wherein step (b) is conducted two, three, four, or five times. 7) The method of any preceding numbered embodiment, further comprising repeating step (c). 8) The method of numbered embodiment 7, wherein step (c) is conducted two, three, four, or five times. 9) The method of any preceding numbered embodiment, further comprising discarding the portion of the solubilized monosaccharides and/or disaccharides removed in step (b). 10) The method of any preceding numbered embodiment, wherein the portion of the solubilized monosaccharides and/or disaccharides removed in step (b) is not combined with the portion of the one or more oligosaccharides of step (e) to form the ingredient. 11) The method of any preceding numbered embodiment, wherein the ingredient is substantially free of monosaccharides. 12) The method of any preceding numbered embodiment, wherein the ingredient is substantially free of disaccharides. 13) The method of any preceding numbered embodiment, wherein the one or more oligosaccharides comprise at least one of: i) a cello-oligosaccharide having a degree of polymerization (DP) of from two to six; ii) a xylo-oligosaccharide having a DP of from two to twelve; iii) an arabinoxylo-oligosaccharide having a DP of from three to fifteen; iv) a manno-oligosaccharide having a DP of from two to twelve; v) a mixed-linkage glucan oligosaccharide having a DP of from two to five; vi) a xyloglucan oligosaccharide having a DP of from four to twelve; or vii) a chito-oligosaccharide having a DP of from two to twelve. 14) The method of numbered embodiment 13, wherein the ingredient comprises at least two of the oligosaccharides listed in (i) to (vii). 15) The method of numbered embodiment 14, wherein the ingredient comprises the at least two oligosaccharides in a ratio from 1:9 to 1:1 in relation to each other. 16) The method of any preceding numbered embodiment, wherein the monosaccharides and/or disaccharides comprise at least one of sucrose, glucose, maltose, lactose, glucose, fructose, or galactose. 17) The method of any preceding numbered embodiment, wherein the physically treating of step (a) comprises at least one of chipping, chopping, milling, ball-milling, grinding, sprucing, or blending the plant biomass. 18) The method of any preceding numbered embodiment, wherein the incubating of step (b) occurs in an incubation solution comprising water. 19) The method of any preceding numbered embodiment, wherein the incubating of step (b) occurs at a temperature of from 15° C. to 95° C. 20) The method of any preceding numbered embodiment, wherein the incubating of step (b) is conducted from 15 minutes to 1 hour. 21) The method of any preceding numbered embodiment, wherein the thermochemically treating of step (c) comprises heating the physically treated plant biomass in the acidic solution or the alkali solution. 22) The method of numbered embodiment 21, wherein the heating is at a temperature of from 50° C. to 150° C. 23) The method of numbered embodiment 21 or 22, wherein the heating is conducted from 30 minutes to 4 hours. 24) The method of any preceding numbered embodiment, wherein the incubated plant biomass is thermochemically treated in an alkali solution having a pH from 8 to 11. 25) The method of numbered embodiment 24, wherein the alkali solution comprises at least one of sodium hydroxide, potassium hydroxide, sodium carbonate, calcium carbonate, aqueous ammonia, ammonium sulfate, or ammonium hydroxide. 26) The method of any preceding numbered embodiment, wherein the incubated plant biomass is thermochemically treated in an acidic solution having a pH from 4 to 6. 27) The method of numbered embodiment 26, wherein the acidic solution comprises at least one of sulfuric acid, hydrochloric acid, nitric acid, phosphoric acid, acetic acid, maleic acid, fumaric acid, or oxalic acid. 28) The method of any preceding numbered embodiment, wherein the plant biomass comprises at least one of sugar cane, corn stover, corncob, wheat bran, wheat straw, hardwood, or softwood. 29) The method of any preceding numbered embodiment, wherein the plant biomass comprises at least one of cellulose, chitin, chitosan, xylan, xyloglucan, mixed-linkage glucan, mannan, or lignocellulose. 30) The method of any preceding numbered embodiment, wherein the one or more polysaccharide-cleaving enzymes comprises at least one of cellulase, xylanase, xyloglucanase, endo-glucanase, cellobiohydrolase, mannanase, lichenase, or lytic polysaccharide monooxygenase (LPMO). 31) The method of any preceding numbered embodiment, wherein the one or more polysaccharide-cleaving enzymes comprises at least one of AA9, AA10, AA11, AA13, AA14, or AA15. 32) The method of any preceding numbered embodiment, wherein the one or more of the polysaccharide-cleaving enzymes is prepared from Trichoderma reesei fungi. 33) The method of any preceding numbered embodiment, wherein the one or more polysaccharide-cleaving enzymes is operably linked to a catalytic module. 34) The method of any preceding numbered embodiment, wherein the one or more polysaccharide-cleaving enzymes is operably linked to a non-catalytic module. 35) The method of numbered embodiment 34, wherein the non-catalytic module is a carbohydrate-binding module. 36) A method for producing an ingredient for human consumption, the method comprising: (a) pretreating a plant biomass, wherein the pretreating comprises: (i) physically treating the plant biomass; (ii) incubating the plant biomass in an incubation solution having a pH from 6.6 to 7.4 to solubilize a portion of the monosaccharides and/or disaccharides and removing a portion of the solubilized monosaccharides and/or disaccharides; and (iii) thermochemically treating the plant biomass in one of (i) an acidic solution having a pH from 2 to 6.5 or (ii) an alkali solution having a pH from 7.5 to 12; (b) contacting, in a solution or suspension, one or more polysaccharide-cleaving enzymes and the pretreated plant biomass to form one or more oligosaccharides; and (c) isolating a portion of the one or more oligosaccharides to form the ingredient.

### EXAMPLES

The following illustrative examples are representative of embodiments of the compositions and methods described herein and are not meant to be limiting in any way.

### Example 1—Exemplary Process

The following steps can be performed to generate an ingredient as provided herein:
1. Physical pretreatment of a plant biomass: Mix 100 g of milled corncob with water to a 10% (w/w) solids concentration and mix for 60 minutes at room temperature. At the end of the 60 minutes stop mixing and filter out the liquid, while keeping the solids.
2. Re-suspend the solids in water at a concentration of 10% (w/w) solids in suspension. Start mixing, heat to 95° C., and mix for 60 minutes at 95° C.
3. At the end of 60 minutes of heating, add 6 g of sodium hydroxide (0.2-3% by weight of corncob) and continue stirring. Heat to a temperature of 95° C. and mix for 60 minutes to break down the hemicelluloses present in corncob. At the end of the 60 minutes, stop heating and cool down to a temperature of 50° C.
4. Hydrolysis: Add cellulolytic enzymes (e.g., from Trichoderma reesei) and incubate at 50° C. at pH 5.5 (adjusted by 1 mol/L sulfuric acid and/or 1 mol/L sodium hydroxide) for 72 hours.
5. Separation of the biomass: At the end of the hydrolysis, separate the liquid from the products through a solid-liquid separator.
6. Enzyme separation following enzymatic hydrolysis: the liquid fraction from the slurry includes enzymes, oligosaccharides, water, and salts that need to be separated. Use a 10 kDa hollow fiber membrane to separate the enzyme proteins and other macromolecules.
7. Salts are removed using ion-exchange columns at ≤45° C.
   a. Cation column: Strongly acidic cation exchange resin, cross-linked polystyrene matrix, sulfonate functional group, Na⁺ counter-ion.
   b. Anion column: Macroporous, weakly basic anion exchange resin, cross-linked polystyrene matrix, dimethyl-tertiary amine functional group, OH counter-ion.
8. Oligosaccharides concentration: The desired oligosaccharides are selectively concentrated through nanofiltration at room temperature.
9. Concentration: Concentrate the liquid to 40-75% at 60-80° C.
10. Spray drying: Spray dry with inlet temperatures of 130-160° C. and outlet temperatures of 65-85° C.

### Example 2—Removal of Soluble Saccharides by a Washing Cycle (i.e., Incubation Cycle)

The following steps were performed to remove soluble saccharides from plant biomass:
1. Water was added to 100 mg of four plant biomass types (i.e., cane, wheat, cob, and willow) to a concentration of 10% (w/v) and incubated at 45° C. for 30 minutes, after which suspensions were centrifuged and supernatants were removed.
2. Step 1 was repeated 5 times.
3. 2.5 μl of each supernatant fraction was analyzed by thin-layer chromatography (TLC).

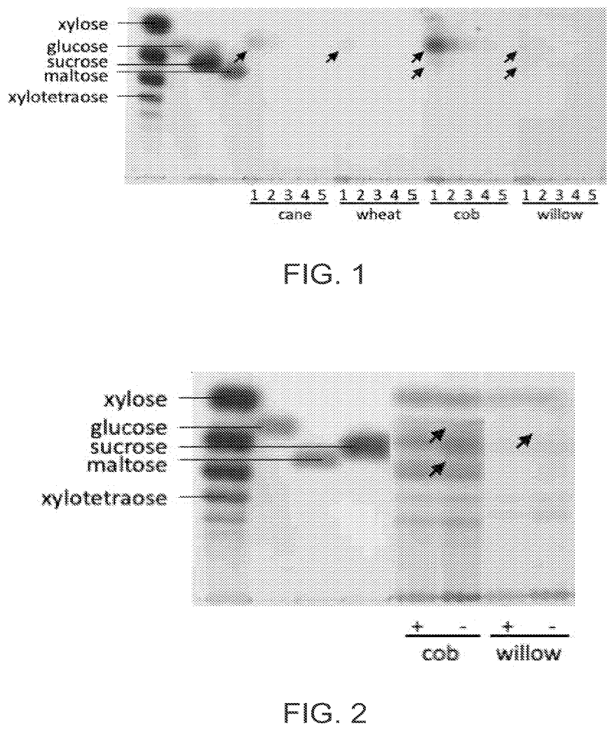

The thin-layer chromatogram of FIG. 1 shows the presence of soluble saccharides washed from four types of plant biomass in the five sequential washing cycles or incubation cycles (1, 2, 3, 4, and 5). Undesired monosaccharides and disaccharides, such as glucose, sucrose, and maltose are arrowed. The results of the TLC showed that for all plant biomasses, the supernatants removed after the first washing cycles have abundant monosaccharides and disaccharides present in them. Thus, the washing cycle successfully removed the monosaccharides and disaccharides from the plant biomass. Supernatants from subsequent washing cycles have significantly fewer monosaccharides and disaccharides present in them, if any at all, showing that the plant biomass has minimal monosaccharides and disaccharides remaining in it after a washing cycle as provided herein.

### Example 3—Absence of Washed Soluble Saccharides in Enzyme Hydrolyses

The following steps were performed to show the absence of soluble saccharides in enzyme hydrolyses:
1. The washed cob and willow plant biomasses from Example 2 were each incubated in 1% (w/v) NaOH at 99° C. for 30 minutes and then cooled. 100 mg of unwashed cob and willow biomasses were also each incubated in 1% (w/v) NaOH at 99° C. for 30 minutes and then cooled.
2. 150 μl of the resulting suspensions were each mixed with 150 μl 1M ammonium acetate (pH 5.5) and 150 μl of an enzyme composition comprising beta xylanase and cellobiohydrolase. Suspensions were then incubated at 50° C. for 16 hours to allow the enzyme reactions with the plant biomasses to occur.
3. 2.5 μl of each supernatant fraction was analyzed by TLC.

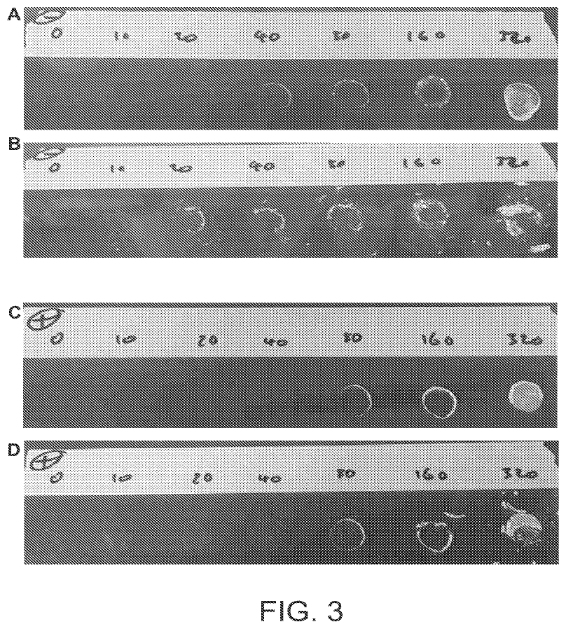

The thin layer chromatogram of FIG. 2 shows the products of enzyme digestion of the four types of biomass that have (+) or have not (−) been washed as in Example 2. The results show that after enzyme digestion the end products included glucose, sucrose, and maltose for the unwashed cob and willow plant biomasses. However, the end products after enzyme digestion did not include glucose, sucrose, and maltose for the washed cob and willow plant biomasses.

### Example 4—Adding Polymer to Oligosaccharide Solutions Enables them to Dry into a Hard Glaze

The following steps were performed to show that adding polymer to oligosaccharide solutions enables them to dry into a hard glaze:
1. 100 μl of 10-320 mM (10, 20, 40, 80, 160, and 320 mM) cellobiose+1% w/v birchwood xylan was pipetted onto a glass plate.
2. Samples were dried at 37° C.
3. Samples were scored with a knife to test whether or not the ingredient settled into a solid glaze. With cellobiose alone, no firm glaze was formed, and the dried powder readily cracked when pressure was applied with a knife. In contrast, the composition with 1% w/v xylan added and with cellobiose at 80 mM or less the composition dried to form a solid, off-white, translucent surface that was strong enough to be scored with a knife leaving an indentation but without cracking. With a cellobiose concentration of 160 mM or higher (5.5% w/v, or 550% w/w as compared with xylan) the morphology of the glaze reverted to that without xylan present. That is, no firm glaze was formed, and the dried powder readily cracked when pressure was applied with a knife (see FIG. 3).

### Example 5—Demonstration of a Composition Comprising Two Oligosaccharides and a Polysaccharide in Food Products

The following steps were performed to demonstrate a composition comprising two oligosaccharides and a polysaccharide in food products:
1. 4 g birchwood xylan was dissolved in 75 ml water with boiling.
2. 12 g cellobiose and 24 g xylo-oligosaccharides (primarily degree of polymerization (DP) 2-6) was added in 3 g increments and dissolved with boiling.
3. The mixture was reduced to 50 ml with heating and formed a thick solution with the consistency and appearance of cloudy honey but less sweet.
4. 10 mL of the mixture was mixed with 12 g oats to make a flapjack/cereal bar mixture and separately 10 mL of the mixture was also mixed with 6 g fruit and 6 g nuts to make a cereal bar mixture.
5. Samples were baked at 100° C. for 10 minutes and then left to cool and dry overnight.

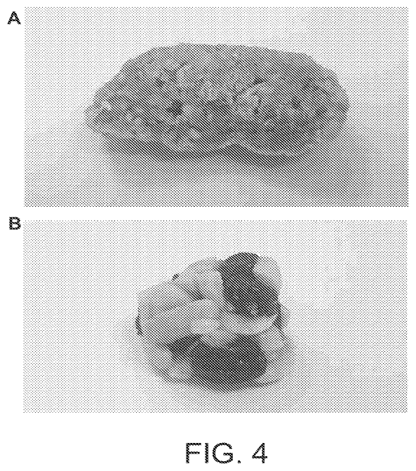

As shown in FIG. 4, panel A (flapjack/cereal bar) and panel B (fruit and nut bar), the produced flapjack and fruit and nut bar were of a desirable texture and consistency in line with flapjack/cereal bars and fruit and nut bars made with known syrups typically used in baking.

For the flapjack/cereal bar, the thick solution from step 3 helped to bind together the mixture in step 4. The combined effect of the ingredient with the properties of oats yielded a grainy surface texture consistent with flapjack/cereal bars produced using conventional sugar. The ingredient created a firm, chewy, moist, viscous texture yielding a mouthfeel consistent with what is expected from these types of food products, but which is not present in oats alone. The product was mildly sweet and contained no bitter or off-flavors, that can be characteristic of high-intensity sweeteners.

For the fruit and nut bar, the thick solution from step 3 helped to bind together the mixture in step 4. It also added a smooth, shiny glazed surface, which is a core part of the aesthetic qualities of such food products, and which would not be created if the ingredient comprised oligosaccharides alone (i.e., oligosaccharides without the polysaccharides). The ingredient created a firm, chewy, moist, and viscous texture yielding a mouthfeel consistent with what is expected from these types of food products, but which is not present in either the nuts or the fruit alone. The product was mildly sweet and contained no bitter or off-flavors, that can be characteristic of high-intensity sweeteners.

### Example 6—Process for Making an Ingredient

The following steps can be performed to make an ingredient as provided herein:
1. Heat 15% w/v of corncob (ground through a 1 mm pore size filter) in 1% w/v NaOH for 1 hour at 90° C., thereby solubilizing a portion of the polymeric components of the biomass.
2. Adjust to pH 5.5 with sulfuric acid.
3. Extract a volume comprising only liquid components of the reaction representing 15% of the total volume of the reaction (“soluble polymers”); retain the remaining 85% volume of the reaction comprising all of the insoluble biomass fraction (“remaining biomass”).
4. To the remaining biomass, add a cellulolytic enzyme cocktail (e.g., an enzyme cocktail from Trichoderma reesei, including cellulase, xylanase, arabinofuranosidase, LPMO, etc.) to 0.5% w/v and incubate at 50° C. for 24 hours.
5. Separate soluble oligomeric reaction products from undigested insoluble polymeric compounds by filtration.
6. Purify oligomeric reaction products by sequentially employing microfiltration, ultrafiltration, and ion-exchange chromatography (e.g., cross-flow filtration on a ceramic membrane; filtration can be performed using 110 0.45 μm cut-off Inside Céram candle filters (TiO2, Ø 25 mm×L 1178 mm HD 6 mm, 8 channels per membrane) supplied by TAMI industries at a feed pressure of maximum 3 bar).
7. Purify the soluble polymers by employing ultrafiltration (e.g., 10 kDa spiral wound membranes (Snyder ST-2B-6338, PES, feed spacer thickness 31 mm) run on Alfa Laval ultrafiltration unit).
8. Recombine the solutions formed in steps 6 and 7 and further purify and concentrate by employing nanofiltration to form the ingredient.

### Example 7—Viscosity Measurements of Different Solutions

The following steps were performed to measure viscosity of different solutions:
1. Three saccharide solutions comprising cellobiose (Cell₂), xylo-oligosaccharides of primarily DP 2-6 (XOS), and polymeric beechwood xylan (BWX) were created by boiling saccharides in water. Final concentrations were:
   a. Sample 1:0.33 g/ml Cell₂, 0.66 g/ml XOS, 0.13 g/ml BWX
   b. Sample 2:0.17 g/ml Cell₂, 0.33 g/ml XOS, 0.07 g/ml BWX;
   c. Sample 3:0.54 g/ml Cell₂, 0.52 g/ml XOS, 0.07 g/ml BWX.
2. The samples were tested using a Brookfield HDB VE roto-viscometer using standard testing procedures. A 400 mL sample was taken in a tall-form beaker to ensure that no container effects occurred. The instrument was operated as per the manufacturer's instructions with respect to ranges: rotoviscometry using spindle code 61, spindle speed 100 rpm, and at 22° C.

| Sample number | Viscosity (cps) |
|---|---|
| 1 | 393 |
| 2 | 13 |
| 3 | 26 |

Sample 1 had a consistency like that of thick honey that needed mixing in order to dilute into aqueous solutions. In contrast, Samples 2 and 3 were much runnier and could be readily mixed into aqueous solutions. The results showed that the viscosity of the compositions was affected more by the polysaccharide concentration than the overall concentration of oligosaccharide and polysaccharide. Samples 2 and 3 have the same polysaccharide concentration, but Sample 3 has twice the concentration of total oligosaccharide and polysaccharide than Sample 2. The viscosity of Sample 3 is twice the viscosity of Sample 2, in line with a linear relationship between the overall concentration and viscosity. However, there is an exponential increase in the viscosity values as the concentration of the polysaccharide increases. The polysaccharide concentration of Sample 1 is twice that of Sample 3 and their concentrations of total oligosaccharide and polysaccharide are the same, yet the viscosity of Sample 1 is fifteen (15) times greater than Sample 3.

### Example 8—Preparation of Water-Soluble Liquid Product/Ingredient

The following steps were performed to generate a water-soluble liquid product/ingredient (Sample 4):
1. 100 g of milled corncobs were heated in 1 L of deionized water containing 2.5 g of sodium chlorite at 80° C. for 1.5 hours with constant agitation. The residual volume was reconstituted to 900 mL by addition of 200 mL deionized water containing a further 5 g of sodium chlorite and heated at 80° C. for 1 hour with constant agitation.
2. The solution was filtered through a 2 mm pore size ceramic filter funnel with vacuum, until the filtrate was clear.
3. Retained solids were incubated in 1 L 0.5 M sodium hydroxide 0.1% (w/v) sodium borohydride for 17 hours at 50° C. and 115 rpm shaking.
4. The pH was then adjusted to 7 with concentrated sulfuric acid and dialyzed against tap water for 24 hours in 12,000 Dalton cut-off dialysis tubing.
5. The contents of the dialysis tubing were transferred to a 2-L beaker and the insoluble fraction allowed to sediment by gravity.
6. The supernatant was decanted twice and concentrated by evaporation at 80° C. to a volume of 120 mL.
7. Water-soluble polymer was precipitated by centrifugation after addition of 3 volumes of ethanol. The resulting supernatant was discarded and the precipitate air dried to constant weight at room temperature.
8. Oligosaccharides were added to a final w/w of 10% cellobiose, 75% xylo-oligosaccharides, 15% extracted water-soluble polymer, and mixed to homogeneity in a Waring Xtreme blender on the lowest power setting. 94 g solids were recovered from the blender.
9. Unexpectedly, all 94 g solids dissolved in 60 mL water at 50° C. with mild constant agitation (˜100 rpm), indicating a solubility of greater than 150 g/100 g.

The sample generated (e.g., at steps 8 and 9 above) is referred to as Sample 4.

### Example 9—Physicochemical Properties of the Water-Soluble Liquid Product of Example 8

Flow characteristics: The flow characteristics of the water-soluble liquid product in accordance with the present disclosure (Sample 4) described in Example 8 are detailed in Table 1 along with comparison compositions of water, 20% w/v glucose, 40% w/v glucose, 60% w/v glucose, 80% w/v glucose, and ≥99% glycerol (Fisher G/0650/17 as supplied). The glucose solutions were made by weighing 6, 12, 18, and 24 g D-glucose respectively and making up with 90° C. water to 30 mL. The flow characteristics were measured by timing the flow rate of 5 mL, and where suitable, 20 mL of the liquids from a vertically stood syringe (BD Plastipak 300613) filled with 20 mL of test liquid under gravity at room temperature.

TABLE 1
Flow Characteristics

| | Time for 5 mL flow | Time for 20 mL flow |
|---|---|---|
| Sample | (seconds) | (seconds) |
| Water | 2 | 13 |
| 20% w/v glucose | 2.3 | 13.5 |
| 40% w/v glucose | 2.5 | 15 |
| 60% w/v glucose | 3 | 20 |
| 80% w/v glucose | 5 | 38 |
| ≥99% Glycerol | 256 | Not Determined |
| Sample 4 | 237 | Not Determined |

The increased time taken for a sample to flow out of the bottom of the syringe (i.e., has a lower flow rate) correlates with increased viscosity of the sample. The lower the flow rate, the more syrup-like/sticky and viscous the liquid sample is. Measured flow rates for Sample 4 are similar to glycerol and lower than all the glucose solutions tested. This property of Sample 4 makes it more useful than the glucose solutions and water as a binder in foodstuffs, such as cereal bars, as well as providing sweetness to the product.

Color: The color of Sample 4 corresponded to No. 30 by the Standard Reference Method (SRM), a method for color assessment of wort or beer as published in the recommended methods of the American Society of Brewing Chemists (ASBC Methods of Analysis, Beer 10. Spectrophotometric Color Method Approved 1958, rev. 2015. American Society of Brewing Chemists, St. Paul, MN, U.S.A). Briefly, the absorbance of a sample is measured in a cell of path length 1 cm at a wavelength of 430 nm. The resultant absorbance value is multiplied by 12.7 to yield the color value. Given the turbidity of Sample 4, absorbance could not be measured, and an assessment was made by optical comparison to the SRM No. 30 (a dark red/brown color).

Anion exchange chromatography: Analysis of Sample 4 by high-performance anion exchange chromatography (HPAEC) was performed using a Thermo Fisher Scientific DIONEX ICS-6000 system fitted with CarboPac PA200 Analytical column (3×250 mm) and CarboPac PA200G Guard column (3×50 mm) and Dionex ED Electrochemical Detector. Data was acquired with Chromeleon 7 software.

Eluents A (milli Q water), B (250 mM NaOH), and C (250 mM NaOH+1 M Sodium Acetate) were used to produce a mobile phase with the gradient profile shown in Table 2.

TABLE 2
Gradient Profile

| Time | Flow | | | |
|---|---|---|---|---|
| (min) | (ml/min) | % A | % B | % C |
| 0 | 0.5 | 75 | 25 | 0 |
| 3 | 0.5 | 75 | 25 | 0 |
| 6 | 0.5 | 50 | 50 | 0 |
| 15 | 0.5 | 50 | 42.5 | 7.5 |
| 20 | 0.5 | 0 | 0 | 100 |
| 23 | 0.5 | 75 | 25 | 0 |
| 26 | 0.5 | 75 | 25 | 0 |

Sample for analysis was prepared by diluting Sample 4 100-fold and passing through a 0.45 μm syringe filter and the injection volume of analyte was 10 μl.

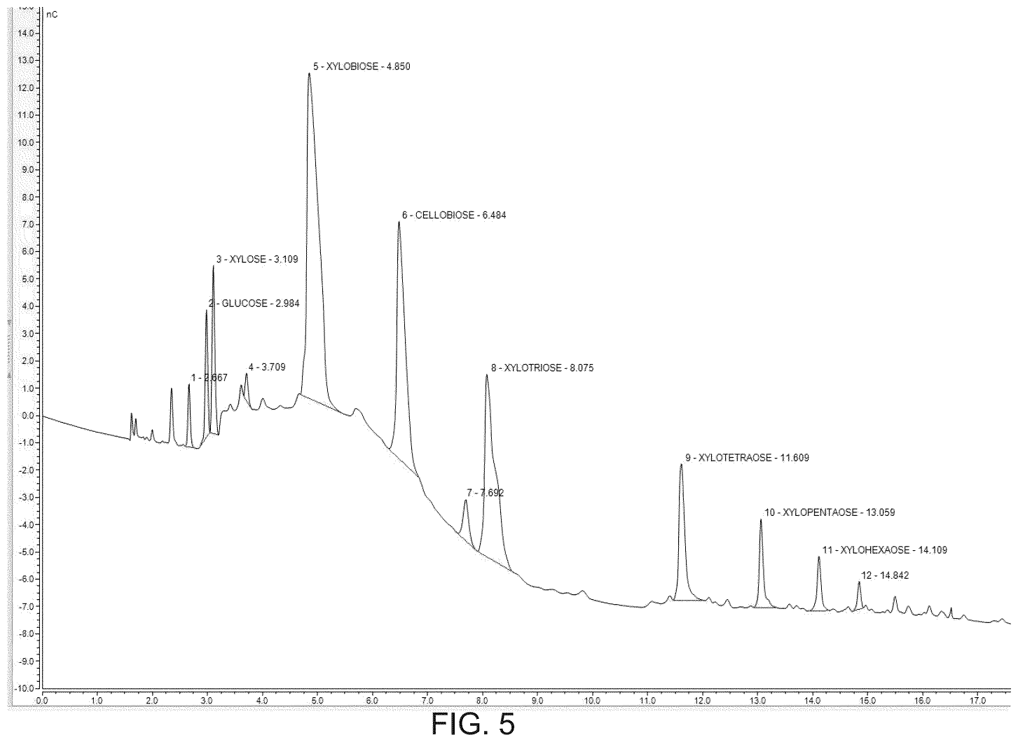

HPAEC analysis (see FIG. 5) confirmed that Sample 4 is a mixture of monosaccharides, disaccharides, and other oligosaccharides composed of glucose and xylose. This is in contrast to syrups typically used in the food industry such as corn syrup and high-fructose corn syrup that contain primarily monosaccharides of glucose and fructose. As a result, the product in Sample 4 when used in foodstuffs is expected to have fewer calories, a lower glycemic index, and contain fiber, in contrast to corn syrup and/or high-fructose corn syrup.

### Example 10—Cold-Pressed Fruit Cereal Bar

A cold-pressed fruit cereal bar was made as follows:
1. 120 g of Sample 4 from Example 8 was heated with 30 g coconut oil, and one-quarter (¼) teaspoon of cinnamon was added.
2. Once foaming, the mixture was removed from the heat and 40 g oats, 40 g dried dates, 10 g of crisped rice, and 10 g seeds were added.
3. The ingredients were mixed thoroughly until all components were coated and the mixture was transferred into a freezer bag and into a freezer. The contents of the bag were rolled to a thickness of 7-10 mm and chilled at 4° C. overnight before cutting into rectangles.

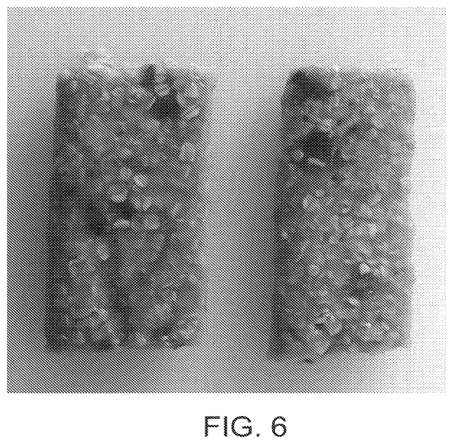

The resulting product (shown in FIG. 6) was a chewy, sticky cereal bar, which was loosely set, and containing oats and crisped rice with perceivable sweetness delivered through Sample 4 and chopped dates.

### Example 11—Producing the Ingredient in a Large Manufacturing Process

The following steps can be used to produce the ingredient in a large manufacturing process:
1. Physical pretreatment of a plant biomass: Mix 100 kg of milled corncob with water at a concentration of 15% (w/w) solids in suspension. Start mixing and heat to 95° C. and mix for 60 min at 95° C. Add 6 kg of sodium hydroxide (0.2-3% by weight of corncob) and continue stirring. Heat to a temperature of 95° C. and mix for 60 minutes to release the hemicelluloses present in corncob. At the end of the 60 minutes, cool down to 50° C. and adjust pH to 5.5 with sulfuric acid.
2. Removal of portion of soluble polysaccharide: Remove of a portion of the soluble phase corresponding to 5-30% of the total xylan. Neutralize with sulfuric acid and concentrate and purify by ultrafiltration. Remove any precipitated polymer.
3. Hydrolysis: Add cellulolytic enzymes (e.g., from Trichoderma reesei) to the milled corncob mixture and incubate at ˜50° C. for 12-72 hours.
4. Separation of the biomass: At the end of the hydrolysis, separate the liquid from the products through a solid-liquid separator such as a filter press or decanting centrifuge.
5. Enzyme separation following enzymatic hydrolysis: The liquid fraction from the slurry contains enzymes, oligosaccharides, water, and salts that need to be separated. Use a 3 kDa or 10 kDa hollow fiber membrane to separate the enzyme proteins and other macromolecules.
6. Salts are removed using ion-exchange columns at ≤45° C.
   a. Cation column: Strongly acidic cation exchange resin, cross-linked polystyrene matrix, sulfonate functional group, and Na⁺ counter-ion.
   b. Anion column: Macroporous, weakly basic anion exchange resin, cross-linked polystyrene matrix, dimethyl-tertiary amine functional group, and OH counterion.
7. Oligosaccharides concentration: The desired oligosaccharides are selectively concentrated through nanofiltration at room temperature.
8. Concentration: Optionally concentrate the liquid to 40-75% at 60-80° C.
9. Recombination: Combine purified soluble polymer with enzyme-yielded oligomers at a dry weights ratio of 5:95-20:80.
10. Spray drying: Spray dry the resultant solution with inlet temperatures of 130-160° C. and outlet temperatures of 65-85° C.

### Example 12—Use of the Liquid Ingredient to Manufacture an Extruded Cereal Bar

The following steps can be performed to use a liquid ingredient as provided herein to manufacture an extruded cereal bar:
1. A 120 kg solution comprising 10.5 kg xylan, 57 kg xylo-oligosaccharides, and 7.5 kg cellobiose is heated with 130 kg coconut oil and transferred into a high-speed mixer. 200 kg rolled oats and 25 kg chopped date/raisin mixture are added and mixed thoroughly. This is pulsed through a dough mixer and set at 20 psi through a dough feed system.
2. The mixture is transferred via belt and ramshorn and baked for 20 minutes at 180° C. The product is then transferred via oven travellator onto a biscuit cutting line and then to variable form fill and seal packaging when cooled to <5° C.
3. The resulting product is a soft, sticky cereal bar that is loosely set and full of oats. Sweetness is delivered through the liquid solution comprising 10.5 kg xylan, 57 kg xylo-oligosaccharides, 7.5 kg cellobiose, and chopped dates in the bar. The liquid solution comprising 10.5 kg xylan, 57 kg xylo-oligosaccharides, 7.5 kg cellobiose, and the coconut oil both act as binders to hold the other ingredients in the bar together and give the bar its structure.

### Example 13—Use of the Liquid Ingredient to Manufacture an Extruded Breakfast Cereal

The following steps can be performed to use a liquid ingredient as provided herein to manufacture an extruded breakfast cereal:
1. Cereal flours (about 85-75% w/v) are combined with a solution comprising 22.5 g xylan, 30 g cellobiose, and 97.5 g xylo-oligosaccharides per 100 g water (about 15-25% v/v), as well as any additives such as preservatives, and vitamins and minerals for fortification to form a dough. This is extruded using a twin screw extruder, which cooks the product using a combination of heat and moisture addition and/or steam and mechanical sheer, forming the product's shape by pushing it through a nozzle. The product is then puffed until light in texture and golden in color and cooled.
2. The result is a light, crispy, shaped breakfast cereal product. The liquid solution comprising 22.5 g xylan, 30 g cellobiose, and 97.5 g xylo-oligosaccharides gives the product sweetness and helps to form the structure of the dough before extrusion.

### Example 14—Use of the Liquid Ingredient to Manufacture a Tomato Ketchup

The following steps can be performed to use a liquid ingredient as provided herein to manufacture a tomato ketchup:
1. 4 onions and 250 g celery are blended until finely chopped in a food processor. They are fried in 5 tbsp vegetable oil on a low heat for 5 minutes. 4 sliced cloves of garlic are added and cooked for a further 5 minutes. 1 tsp ground coriander, 1 short cinnamon stick, 1 tsp all spice, one-half (½) tsp ground black pepper, and 2 tsp celery salt are added and cooked for a further minute.
2. To the mixture, 2 kg ripe, chopped tomatoes, 3 tbsp tomato puree, one-half (½) tsp chili sauce, 200 mL white wine vinegar, and 285 mL of a solution comprising 22.5 g xylan, 30 g cellobiose, and 97.5 g xylo-oligosaccharides per 100 g water, are added. The mixture is brought back to the boil and left uncovered to simmer for an hour until the tomatoes are soft. The cinnamon stick is discarded, the sauce mixture is blended until smooth, and then sieved.
3. The resulting product is a smooth, tangy tomato ketchup. The liquid solution comprising 22.5 g xylan, 30 g cellobiose, and 97.5 g xylo-oligosaccharides sweetens the product and adds body to the sauce, helping to thicken the sauce and bulk the sauce out.

### Example 15—HPAEC Chromatography of Saccharides in Water Post Washing of Corncobs

Corncobs were incubated at 100 g/L in room temperature water (“Wash” in Table 3), then water was decanted. Water was added to original total volume and heated to 90° C. for 60 minutes (“Wetting” in Table 3) before being heated at 90° C. for 60 minutes in dilute NaOH (“Pretreating” in Table 3).

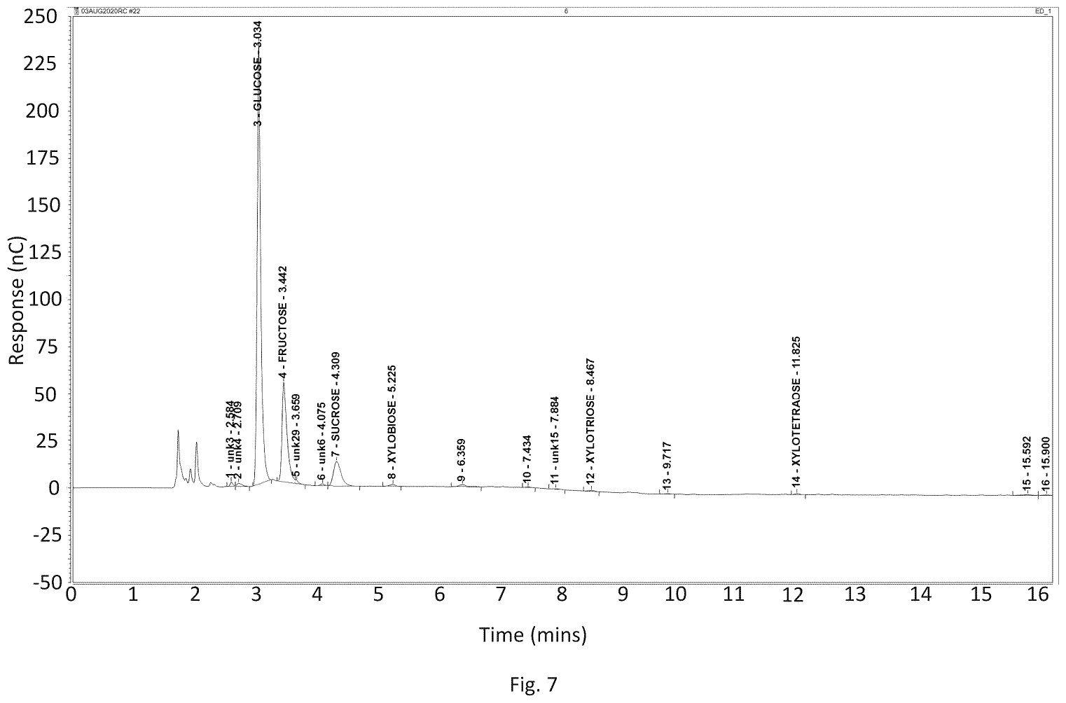

HPAEC was performed on the Wash, Wetting, and Pretreating samples and saccharide peaks were identified (see FIG. 7 for example chromatogram from “Wash”). As indicated in Table 3, about 2% of the corncob at the start of the process is glucose that can be washed out and potentially more glucose may be washed out. It was also noted that the pH after the washing step decreased to 4.5.

TABLE 3

| Step | Glucose g/l |
|---|---|
| Wash | 2.43 g/l |
| Wetting | 1.43 g/l |
| Pretreating | 0.02 g/l |

### Example 16—Quantifying Sugars and Organic Acids

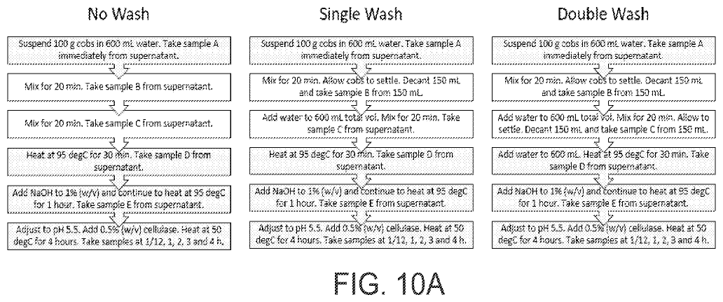

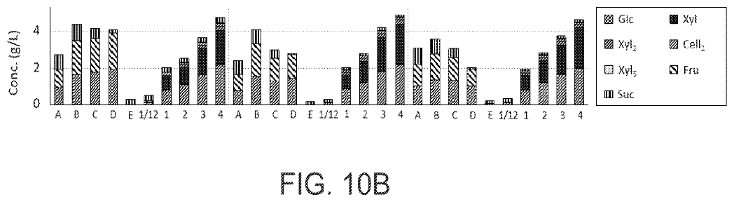

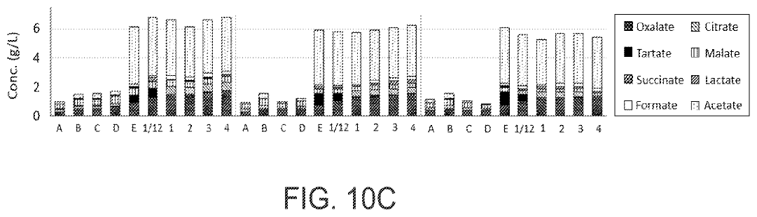

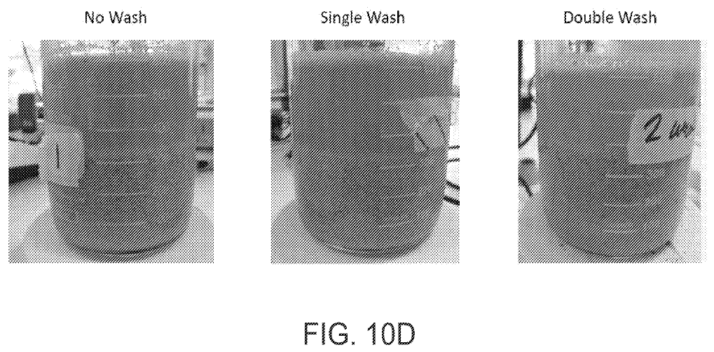

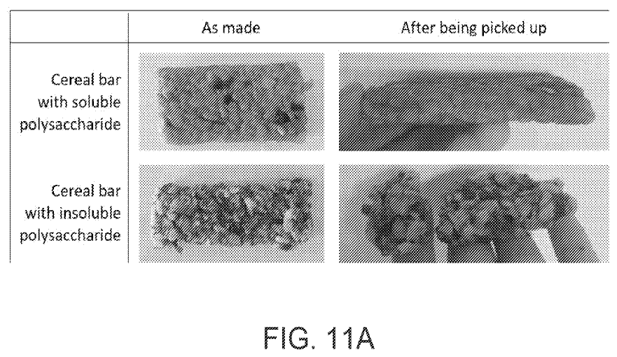

To quantify the impact of the prewashing step on the process, three separate batches of corncobs were treated according to the procedures outlined in FIG. 10A. Samples were analyzed by HPLC for saccharides (FIG. 10B) and HPLC for organic acids (FIG. 10C). The differences in the saccharide and organic acid compositions that were isolated from the different samples (Samples A-E and 5 minutes (1/12 hour) to 4 hours) indicate the impact of washing. The impact of the prewashing may have been greater had more than 150 mL of the 600 mL per wash been extracted. Accordingly, these data indicate the direction that washing can take but not washing's limit.

As shown, glucose, fructose, and sucrose all decreased with prewashing. Fructose and glucose are largely broken down during NaOH treatment, but sucrose is resistant to NaOH treatment. Because sucrose cannot generally be removed from other disaccharides through filtration, it can be useful to remove it by washing. Table 4 shows a comparison of the saccharides in Sample Ds (“No Wash” and “Double Wash”) and Table 5 shows a comparison of saccharides in the four-hour samples (“No Wash” and “Double Wash”).

TABLE 4

| | No Waswh | Double Wash | Change | Change |
|---|---|---|---|---|
| | (g/L) | (g/L) | (g/L) | (%) |
| Glucose | 1.888 | 1.001 | −0.887 | −47.0 |
| Xylose | 0.000 | 0.000 | 0.000 | 0 |
| Xylobiose | 0.000 | 0.000 | 0.000 | 0 |
| Cellobiose | 0.000 | 0.000 | 0.000 | 0 |
| Xylotriose | 0.000 | 0.000 | 0.000 | 0 |
| Fructose | 1.966 | 0.958 | −1.008 | −51.3 |
| Sucrose | 0.232 | 0.045 | −0.188 | −80.63 |

TABLE 5

| | No Waswh | Double Wash | Change | Change |
|---|---|---|---|---|
| | (g/L) | (g/L) | (g/L) | (%) |
| Glucose | 2.132 | 1.987 | −0.145 | −6.8% |
| Xylose | 1.917 | 2.145 | +0.228 | +11.9% |
| Xylobiose | 0.188 | 0.205 | +0.017 | +9.1% |
| Cellobiose | 0.167 | 0.110 | −0.057 | −34.1% |
| Xylotriose | 0.013 | 0.024 | +0.011 | −84.6% |
| Fructose | 0.000 | 0.000 | 0 | 0 |
| Sucrose | 0.292 | 0.125 | −0.167 | −57.19% |

Prior to the start of the “caustic cook” step (e.g., thermochemical step), prewashing lead to a reduction of about 50% of glucose and fructose and about 80% of sucrose. At the end of the hydrolysis, prewashing lead to small differences in small sugars. Xylose-based sugars appear to increase in concentration (as evidenced by the negative change), while glucose and cellobiose decrease in concentration.

A large amount of the organic acids detected are not products of the washing but rather of the pretreatment. However, the unwashed biomass appears to have a higher total loading of organic acids than the two-times washed material.

Acid concentration for the washed biomass also appears lower for the steps leading to the pretreatment stage. Table 6 shows a comparison of the Sample Ds and Table 7 shows a comparison of the four-hour samples.

TABLE 6

| | No Waswh | Double Wash | Change | Change |
|---|---|---|---|---|
| | (g/L) | (g/L) | (g/L) | (%) |
| Oxalate | 0.641 | 0.402 | −0.239 | −37.3% |
| Citrate | 0.186 | 0.092 | −0.094 | −50.5% |
| Tartrate | 0.016 | 0.013 | −0.003 | −18.8% |
| Malate | 0.518 | 0.286 | −0.232 | −44.8% |
| Succinate | 0.063 | 0.002 | −0.061 | −96.8% |
| Lactate | 0.000 | 0.000 | 0 | 0 |
| Formate | 0.000 | 0.000 | 0 | 0 |
| Acetate | 0.295 | 0.003 | −0.292 | −99.0% |

TABLE 7

| | No Waswh | Double Wash | Change | Change |
|---|---|---|---|---|
| | (g/L) | (g/L) | (g/L) | (%) |
| Oxalate | 1.776 | 1.355 | −0.421 | −23.7& |
| Citrate | 0.551 | 0.000 | −0.551 | −100.0% |
| Tartrate | 0.000 | 0.000 | 0 | 0 |
| Malate | 0.403 | 0.240 | −0.163 | −40.4% |
| Succinate | 0.037 | 0.000 | −0.037 | −100.0% |
| Lactate | 0.078 | 0.141 | +0.063 | +80.8 |
| Formate | 0.257 | 0.187 | −0.07 | −27.2% |
| Acetate | 3.727 | 3.512 | −0.215 | −5.8% |

Impact of washing on organic acid content, as shown in “Change” (calculated by “No Wash” minus “Double Wash”), is noticeable both at the end of the wetting stage (Sample D) and at the end of the hydrolysis reaction (4-hour Sample) (see, e.g., Tables 6 and 7). At the end of the wetting stage (Sample D), all other acids except for lactate and formate (n/d) were detected and were at a lower concentration for the material that has been washed twice.

At the end of the hydrolysis, acetate content is higher (hydrolysis releases acetate). Other acids continue to be lower for the washed biomass than the unwashed (with the exception of lactate, which is present in low concentrations).

Visual observation of the samples is shown in FIG. 10D. After two washes, the corncobs released fewer colored compounds and the liquor is lighter. Without being bound by any one particular theory, colored compounds are likely phenols and organic acids released during washing.

### Example 17—Comparison of Cold-Pressed Cereal Bar

Cold-press cereal bars were prepared according to the recipe as before (see Example 10).

Soluble polysaccharides and insoluble polysaccharides were used in the cereal bars for comparison. The soluble and insoluble polysaccharides were as follows:
Soluble polysaccharides: 60 mL water containing 94 g dry ingredient with a composition 10% dry w/w cellobiose, 75% xylo-oligosaccharides, and 15% extracted water-soluble polymer (Sample 4 as described above in Example 8).
Insoluble polysaccharides: 60 mL water containing 94 g dry ingredients with a composition 10% dry w/w cellobiose, 75% xylo-oligosaccharides, and 15% micro-crystalline cellulose.

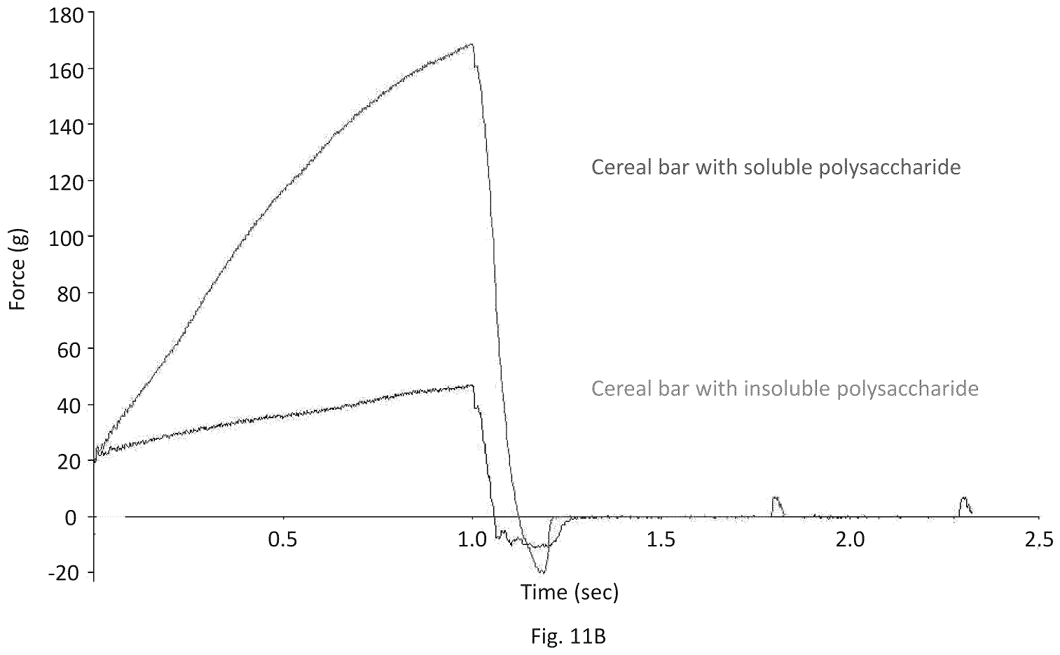

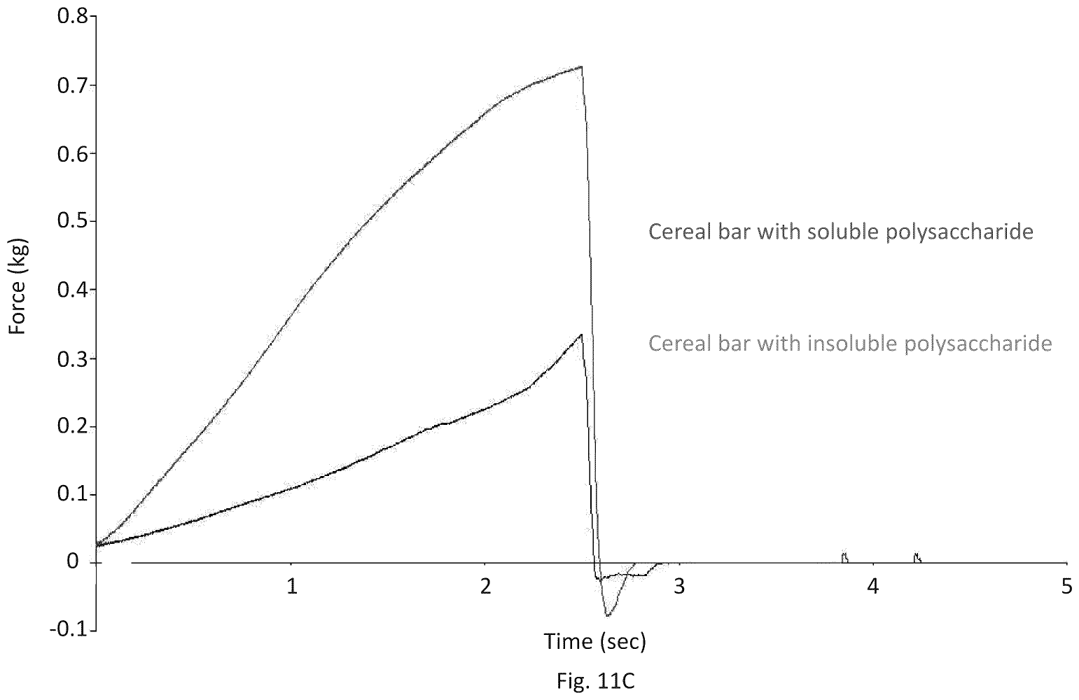

With reference to FIG. 11A, while cereal bars made with insoluble polysaccharide looked like solid bars when placed on the table, they started falling apart as soon as they were lifted from the table and taken in hand due to their soft texture and the ingredients not being bound together well. In contrast, cereal bars made with soluble polysaccharide could be handled with ease and maintained their shape.

Hardness and stickiness of the cereal bars were measured using the TA-XTPlusC Texture Analyser (Stable Microsystems, UK) using the “ExponentC” software. Sample with 9.6 cm×3.8 cm×1 cm dimensions (L×W×H) was placed centrally under the probe. A 6 mm diameter aluminium cylindrical probe was used in a penetration test with “Return To Start” mode and 30 kg load-cell. Once the probe triggered on the surface, it penetrated the 2 mm distance into the sample with 2 mm/s speed. At this point (2 mm depth), the force value was recorded and taken as a measure of “hardness” of the sample. The probe then withdrew from the sample at which point the maximum force to withdraw or “stickiness” was recorded. Pre-test speed was 1 mm/s and post-test speed 10 mm/s.

Results showed that the hardest bar was the one made with soluble ingredient, while lower values were obtained for insoluble ingredient (FIG. 11B). Likewise, the soluble polysaccharide-containing bar had higher stickiness and the insoluble polysaccharide-containing bar lower stickiness. These results confirmed visual and tactile observations.

TABLE 8
Results

| Sample | Hardness (g) | Stickiness (g) |
|---|---|---|
| Cereal bar | 169.19 | −20.33 |
| with soluble | | |
| polysaccharide | | |
| Cereal bar | 47.43 | −11.24 |
| with insoluble | | |
| polysaccharide | | |

TABLE 9
Texture Analyzer Settings

| | Measure Force | |
|---|---|---|
| Mode: | in Compression | |
| Option: | Return To Start | |
| Pre-Test Speed: | 1.0 | mm/s |
| Test Speed: | 2.0 | mm/s |
| Post-Test Speed: | 10.0 | mm/s |
| Distance: | 2 | mm |
| Trigger Type: | Auto - 20 g | |
| Tare Mode: | Auto | |
| Data Acquisition Rate: | 400 | pps |

Hardness of cereal bars was further measured using the TA-XTPlusC Texture Analyser (Stable Microsystems, UK) using the “ExponentC” software. Sample with 9.6 cm×3.8 cm×1 cm (L×W×H) dimensions was placed centrally under the knife. A Knife Edge was used in a cutting test with “Return To Start” mode and 30 kg load-cell. Once the knife triggered on the surface, it penetrated 5 mm into the sample with 2 mm/s test speed. Maximum force measured during the cutting test was recorded as hardness of the sample. Pre-test speed was 1.5 mm/s and post-test speed was 10 mm/s.

Results obtained using the cutting method confirm the results obtained using the penetration method described above. Results show that the hardest bar was the one made with soluble polysaccharides, while lower values were obtained for insoluble polysaccharide-containing bars, the insoluble polysaccharide-containing bar being softer (FIG. 11C).

TABLE 10
Results

| Sample | Hardness (kg) |
|---|---|
| Cereal bar with | 0.73 |
| soluble | |
| polysaccharide | |
| Cereal bar with | 0.34 |
| insoluble | |
| polysaccharide | |

TABLE 11
Texture Analyzer Setting

| | Measure Force | |
|---|---|---|
| Mode: | in Compression | |
| Option: | Return To Start | |
| Pre-Test Speed: | 1.5 | mm/s |
| Test Speed: | 2.0 | mm/s |
| Post-Test Speed: | 10.0 | mm/s |
| Distance: | 5 | mm |
| Trigger Type: | Auto - 25 g | |
| Tare Mode: | Auto | |
| Data Acquisition Rate: | 400 | pps |

### Example 18—Viscosity of Saccharide Composition with Insoluble Polysaccharide Vs. Soluble Polysaccharide

The following samples were prepared:
Sample 1:60 mL water containing 94 g dry ingredients with a composition 10% dry w/w cellobiose, 75% xylo-oligosaccharides, and 15% extracted water-soluble polymer.
Sample 2:60 mL water containing 94 g dry ingredient with a composition 10% dry w/w cellobiose, 75% xylo-oligosaccharides, and 15% micro-crystalline cellulose.

“Sample 4” of Example 8, “Soluble polysaccharides” of Example 17, and “Sample 1” of Example 18 are substantially identical and/or interchangeable. Further, “Sample 2” of Example 18 and “Insoluble polysaccharides” of Example 17 are substantially identical and/or interchangeable.

Sample were analyzed for flow characteristics as described in Example 9. Data confirm that soluble polysaccharides modulate the viscosity of the oligosaccharide composition (Table 12). This can enable fine-tuning of solution viscometric properties in a way not generally possible with oligosaccharide alone.

TABLE 12

| | Time for 5 mL | Time for 20 mL |
|---|---|---|
| Sample | flow (seconds) | flow (seconds) |
| Water | 2 | 13 |
| 20% w/v glucose | 2.3 | 13.5 |
| 40% w/v glucose | 2.5 | 15 |
| 60% w/v glucose | 3 | 20 |
| 80% w/v glucose | 5 | 38 |
| ≥99% Glycerol | 256 | Not Determined |
| Oligosaccharides with | 237 | Not Determined |
| soluble polysaccharide | | |
| (Sample 1) | | |
| Oligosaccharides with | 29 | 58 |
| insoluble polysaccharide | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1.-30. (canceled)

31. A method for producing a composition for human consumption, the method comprising:
(a) subjecting a biomass to a first pretreatment to reduce an average size of the biomass;
(b) subjecting the pretreated biomass to a second pretreatment comprising: incubating the pretreated biomass in an aqueous solution to solubilize monosaccharides and/or disaccharides from the pretreated biomass;
(c) administering a third pretreatment to the second pretreated biomass to increase the digestibility of the biomass, to produce a third pretreated biomass, wherein the third pretreatment is performed after the second pretreatment;
(d) contacting, in a solution or suspension, one or more polysaccharide-cleaving enzymes and the third pretreated biomass to form one or more oligosaccharides;
(e) enriching the solution or suspension to increase the concentration of the one or more oligosaccharides; and
(f) combining the one or more oligosaccharides with polysaccharides, wherein the polysaccharides comprise cellulose, to form the composition.

32. The method of claim 31, wherein the polysaccharides comprise undigested biomass.

33. The method of claim 31, wherein the concentration of polysaccharides in the composition is at least 10% w/w.

34. The method of claim 33, wherein the concentration of polysaccharides in the composition is at least 45% w/w.

35. The method of claim 31, wherein the composition is a nutraceutical product composition.

36. The method of claim 31, wherein the composition is an ingredient for incorporation into a nutraceutical product.

37. The method of claim 36, wherein the ingredient treated in a physical or chemical way before or during incorporation into a nutraceutical product.

38. The method of claim 36, wherein the nutraceutical product is a drink with added dietary fiber, a prebiotic additive, a pill or other capsule.

39. The method of claim 31, wherein the polysaccharides are from the third pretreated biomass.

40. The method of claim 31, wherein the composition has an improved gastrointestinal tolerance, as compared to a saccharide composition comprising primarily monosaccharides and/or disaccharides.

41. The method of claim 31, wherein the composition is a dry composition.

42. The method of claim 31, wherein the combination of the one or more oligosaccharides comprises mixing a first dry form comprising all or part of the one or more oligosaccharides, produced by the drying, lyophilization, or condensation, with a second dry form comprising all or part of the polysaccharides.

43. The method of claim 31, wherein the oligosaccharides comprises a xylo-oligosaccharide having a Degree of polymerization of from two to twelve, and wherein the composition comprises at least 20% by dry weight xylo-oligosaccharides having a degree of polymerization of from two to twelve.

44. The method of claim 31, wherein the second pretreatment further comprises: removing a portion of the solubilized monosaccharides and/or disaccharides from the aqueous solution.

45. A method of making a foodstuff, cosmetic, or nutraceutical product, the method comprising incorporating a dry or liquid composition produced at any stage of the method of claim 31, as an ingredient into the foodstuff, cosmetic or nutraceutical product.
